(19) United States
(12) Patent Application Publication
(10) Pub. No.: US 2021/0246140 A1
(43) Pub. Date: Aug. 12, 2021

(54) MODULATORS OF METABOTROPIC GLUTAMATE RECEPTOR 2

(71) Applicant: The General Hospital Corporation, Boston, MA (US)

(72) Inventors: Zhaoda Zhang, Andover, MA (US); Baohui Zheng, Boston, MA (US); Gengyang Yuan, Boston, MA (US); Ramesh Neelamegam, Boston, MA (US); Anna-Liisa Brownell, Salem, MA (US)

(21) Appl. No.: 17/162,614

(22) Filed: Jan. 29, 2021

Related U.S. Application Data
(60) Provisional application No. 62/968,829, filed on Jan. 31, 2020.

(51) Int. Cl. C07D 471/16; C07D 471/04; C07B 59/00; A61K 51/04

(52) U.S. Cl. CPC C07D 471/16; C07D 471/04; C07B 2200/05; A61K 51/0455; C07B 59/002

(57) ABSTRACT

The present application provides a compound of Formula:

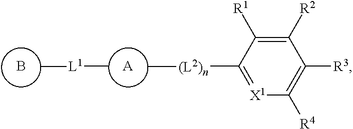

or a pharmaceutically acceptable salt thereof, wherein ring B, L¹, ring A, L², n, R¹, R², R³, R⁴, and X¹ are as described herein. Pharmaceutical compositions comprising the compound, as well as the methods of making and using the compound, are also provided.

### CROSS-REFERENCE TO RELATED APPLICATIONS

This application claims the benefit of priority to U.S. Provisional Application No. 62/968,829, filed Jan. 31, 2020, which is incorporated by reference herein in its entirety.

### GOVERNMENT SUPPORT

This invention was made with government support under Grant Nos. 1R01EB021708 and R01NS100164 awarded by the National Institutes of Health (NIH). The government has certain rights in the invention.

### TECHNICAL FIELD

This disclosure relates to metabotropic glutamate receptor 2 (“mGluR2”) positive allosteric modulators (“PAMs”), and more particularly, to imidazo[4,5-b]pyridine derivatives that can be used as allosteric positron emission tomography (“PET”) imaging probes.

### BACKGROUND

There are numerous deadly diseases affecting current human population. For example, psychiatric and neurodegenerative diseases affect a significant segment of population. As one example, Parkinson's disease (“PD”), a progressive nervous system disorder that affects movement, affects more than 10 million people worldwide with an estimated total annual economic burden of more than $52 billion. In another example, schizophrenia is a long-term mental disorder of a type involving a breakdown in the relation between thought, emotion, and behavior, leading to faulty perception, inappropriate actions and feelings, withdrawal from reality and personal relationships into fantasy and delusion, and a sense of mental fragmentation. Economic burden of schizophrenia in the US exceeds $155 Billion. Currently, there is no cure for these conditions, and only therapeutic approaches that alleviate some of the symptoms are available.

### SUMMARY

Metabotropic glutamate receptor 2 (mGluR2) is an attractive drug discovery target for the treatment of various conditions including, e.g., Alzheimer's disease, schizophrenia, depression, anxiety and pain. A specific mGluR2 radioligand would allow the investigation of mGluR2-related pathophysiology at molecular level using PET. Described herein are compounds that were used as PET radioligands for imaging mGluR2 in rat brain. As an example, compound 1 (FIG. 1) exhibits potent PAM activity (EC₅₀=55 nM), excellent selectivity against other mGlu receptor subtypes (>100-fold), favorable pharmacological and CNS-penetrating properties (e.g., C_{Lint}=17.9 μL/min/mg), Pₑ=9.2×10⁻⁶ cm/s). [¹¹C]1 was conveniently synthesized via O-[¹¹C]methylation of its phenol precursor 1-OH with [¹¹C]methyl iodide. The radiolabeling was achieved with a radiochemical yield of 20±2% (n=10, decay-corrected) based on [¹¹C]CO₂ and a >98% radiochemical purity as well as up to 128 GBq/μmol molar activity at the end of synthesis. The ex vivo biodistribution study demonstrated reversible accumulation of [¹¹C]1 in most tissue areas and hepatobiliary and urinary excretions for radioactivity clearance. In vivo PET imaging studies in rats demonstrated that [¹¹C]1 crossed the blood-brain barrier (BBB) (SUVₘₐₓ=1.8±0.2, n=9) and was mainly accumulated in the mGluR2-rich regions of thalamus, striatum, cerebellum and cortex. Pre-administration of mGluR2-selective PAM (compound 3, JNJ-46356479, 4 mg/kg, iv) reduced the brain uptake of [¹¹C]1, indicating a specific and reversible binding to an mGluR2 allosteric site in rat brain. Pre-administration of the compound 1, as a PAM, had a significant pharmacological effect on [¹¹C]1 accumulation enhancing it (max. 50%) in all investigated brain areas. Therefore, [¹¹C]1 is not only a suitable PET imaging ligand for mGluR2 in the brain but also a therapeutic agent for the treatment of CNS disorders due to its strong activating effects.

In some embodiments, the present disclosure provides a compound of Formula (I):

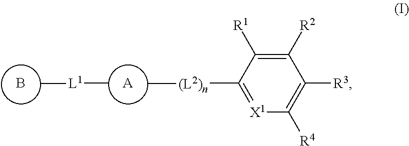

or a pharmaceutically acceptable salt thereof, wherein:
ring B is selected from formula (i), formula (ii), formula (v), and formula (vi):

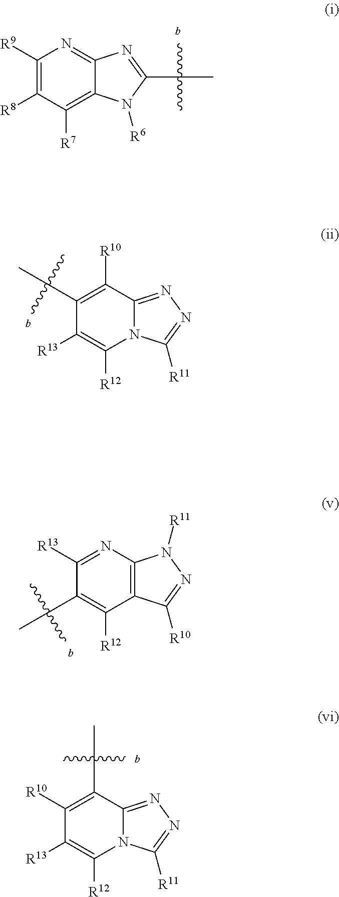

wherein b indicates a point of attachment of ring B to L¹;
L¹ is C₁₋₃ alkylene, which is optionally substituted with 1 or 2 substituents independently selected from halo, C₁₋₃ haloalkyl, OH, C₁₋₃ alkoxy, C₁₋₃ haloalkoxy, amino, C₁₋₆alkylamino, di(C₁₋₆ alkyl)amino, thio, and C₁₋₆ alkylthio;
ring A is selected from formula (iii) and formula (iv):

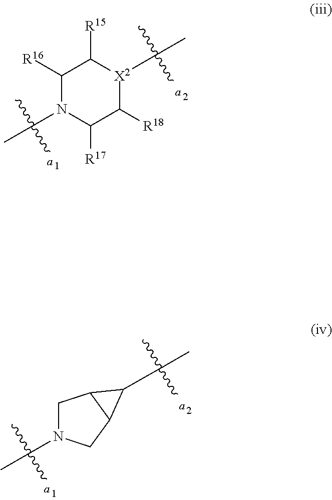

wherein a₁ indicates a point of attachment of ring A to L¹, and a₂ indicates a point of attachment of ring A to L²;
each L² is independently selected from C₁₋₃ alkylene, O, N(R^{N}), and S(═O)₂, wherein said C₁₋₃ alkylene is optionally substituted with 1 or 2 substituents independently selected from halo, C₁₋₃ haloalkyl, OH, C₁₋₃ alkoxy, C₁₋₃ haloalkoxy, amino, C₁₋₆alkylamino, di(C₁₋₆alkyl)amino, thio, and C₁₋₆ alkylthio;
each R^{N} is selected from H and C₁₋₃ alkyl;
n is 0, 1, 2, or 3;
X¹ is selected from N and CR⁵;
X² is selected from N and CR¹⁴;
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆alkylamino, di(C₁₋₆ alkyl)amino, thio, and C₁₋₆ alkylthio; and
or R⁶ and R⁷, together with the carbon atom to which R⁷ is attached and the N atom to which R⁶ is attached form a 5-7-membered heterocycloalkyl ring, which is optionally substituted with 1, or 2 substituents independently selected from OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆alkylamino, di(C₁₋₆alkyl)amino, thio, and C₁₋₆ alkylthio;
provided that the compound of Formula (I) comprises at least one radioisotope selected from ¹¹C and ¹⁸F,
and further provided that the compound of Formula (I) is not any of the following compounds:

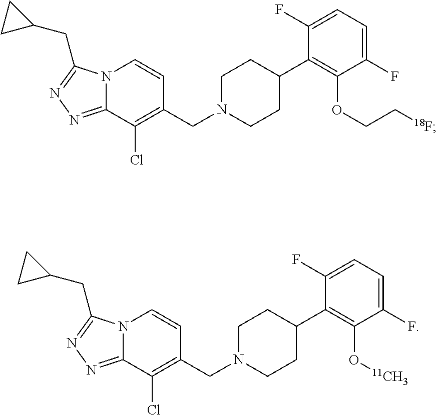

In some embodiments of Formula (I):

L¹ is C₁₋₃ alkylene;
each L² is independently selected from C₁₋₃ alkylene, O, and N(R^{N});
n is 0, 1, or 2; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, halo, C₁₋₃ alkyl, C₁₋₃ alkoxy, C₁₋₃ haloalkyl, C₁₋₃ haloalkoxy, and C₃₋₁₀ cycloalkyl-C₁₋₃ alkyl.

In some embodiments, the compound of Formula (I) has formula:

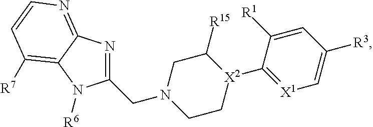

or a pharmaceutically acceptable salt thereof, wherein:
R¹⁵ is selected from H and C₁₋₃ alkyl;
X¹ is selected from N and CH;
X² is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl, C₁₋₃ haloalkyl, or HO—C₁₋₃ alkyl; and
R⁷ is H, or R⁷ and R⁶ together with the atoms to which they are attached form a 6-membered heterocycloalkyl ring.

In some embodiments of Formula (I), the compound is selected from:

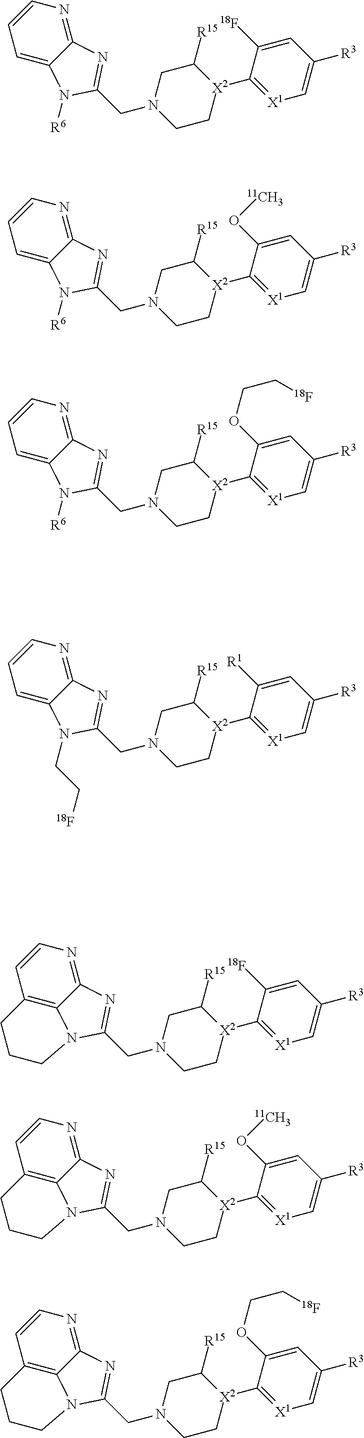

or a pharmaceutically acceptable salt thereof.

In some embodiments, the compound of Formula (I) has formula:

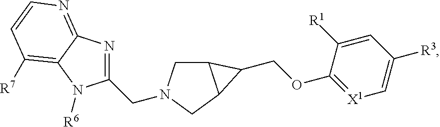

or a pharmaceutically acceptable salt thereof, wherein:
X¹ is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl; and
R⁷ is H, or R⁷ and R⁶ together with the atoms to which they are attached form a 6-membered heterocycloalkyl ring.

In some embodiments of Formula (I), the compound is selected from any one of the following compounds:

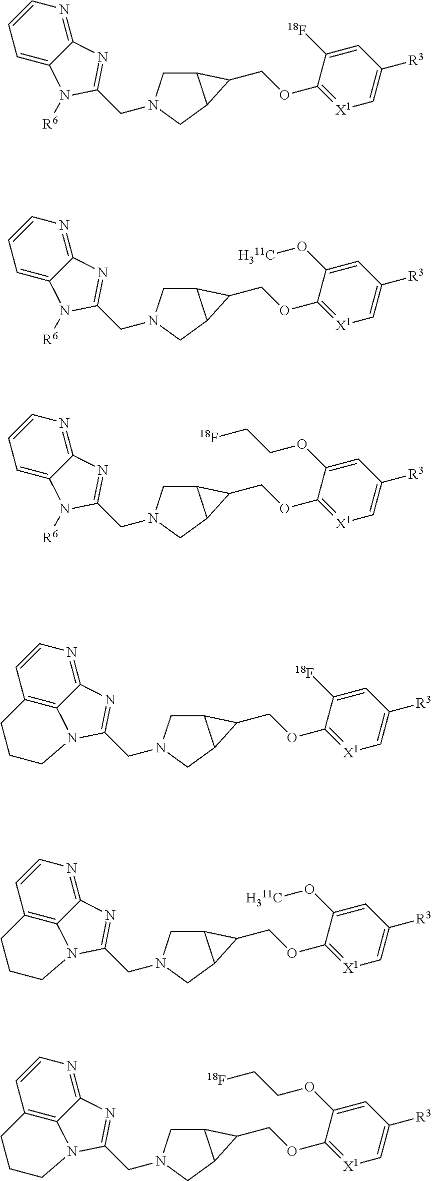

or a pharmaceutically acceptable salt thereof.

In some embodiments, the compound of Formula (I) has formula:

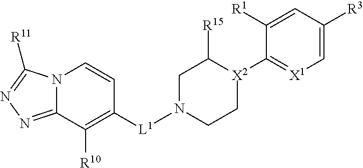

or a pharmaceutically acceptable salt thereof, wherein:
L¹ is selected from CH₂ and C(═O);
   R¹¹ is C₃₋₁₀ cycloalkyl-C₁₋₃ alkyl;
   R¹⁰ is selected from halo and C₁₋₃ haloalkyl;
   R¹⁵ is selected from H and C₁₋₃ alkyl;
   X¹ is selected from N and CH;
   X² is selected from N and CH; and
   R¹ and R³ are each independently selected from halo, C₁₋₃ alkoxy, C₁₋₃ haloalkyl, and C₁₋₃ haloalkoxy.

In some embodiments of Formula (I), the compound is selected from any one of the following compounds:

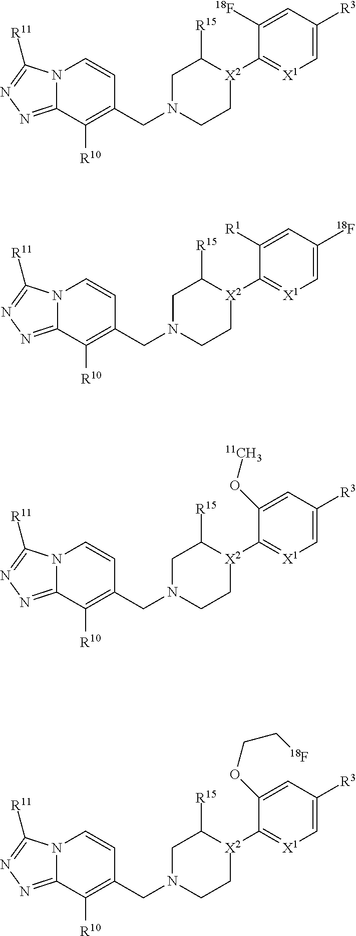

or a pharmaceutically acceptable salt thereof.

In some embodiments of Formula (I), the compound is selected from any one of the following compounds:

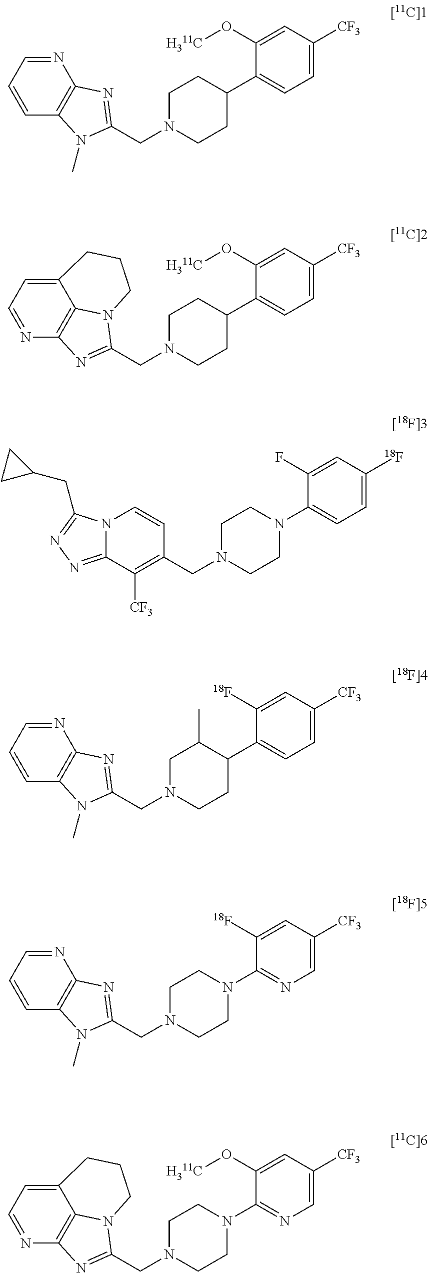 [SMILES: Cn1c(CN2CCC(c3ccc(C(F)(F)F)cc3O[11CH3])CC2)nc2ncccc21 ; [11CH3]Oc1cc(C(F)(F)F)ccc1C1CCN(Cc2nc3nccc4c3n2CCC4)CC1 ; Fc1cc([18F])ccc1N1CCN(Cc2ccn3c(CC4CC4)nnc3c2C(F)(F)F)CC1 ; CC1CN(Cc2nc3ncccc3n2C)CCC1c1ccc(C(F)(F)F)cc1[18F] ; Cn1c(CN2CCN(c3ncc(C(F)(F)F)cc3[18F])CC2)nc2ncccc21 ; [11CH3]Oc1cc(C(F)(F)F)cnc1N1CCN(Cc2nc3nccc4c3n2CCC4)CC1]

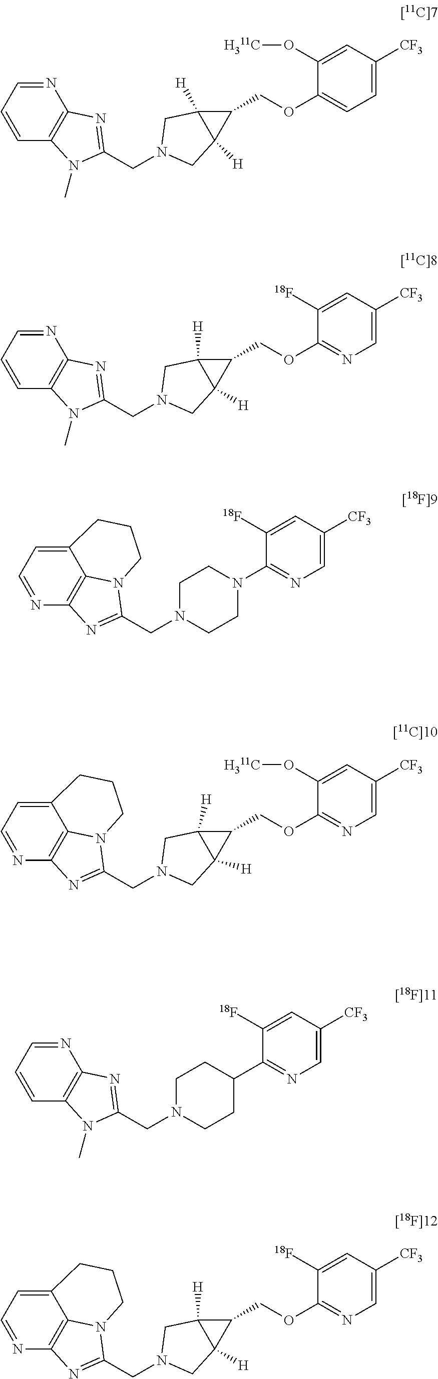 [SMILES: Cn1c(CN2C[C@@H]3[C@@H](COc4ccc(C(F)(F)F)cc4O[11CH3])[C@@H]3C2)nc2ncccc21 ; Cn1c(CN2C[C@@H]3[C@@H](COc4ncc(C(F)(F)F)cc4[18F])[C@@H]3C2)nc2ncccc21 ; FC(F)(F)c1cnc(N2CCN(Cc3nc4nccc5c4n3CCC5)CC2)c([18F])c1 ; [11CH3]Oc1cc(C(F)(F)F)cnc1OC[C@@H]1[C@H]2CN(Cc3nc4nccc5c4n3CCC5)C[C@@H]12 ; Cn1c(CN2CCC(c3ncc(C(F)(F)F)cc3[18F])CC2)nc2ncccc21 ; FC(F)(F)c1cnc(OC[C@@H]2[C@H]3CN(Cc4nc5nccc6c5n4CCC6)C[C@@H]23)c([18F])c1]

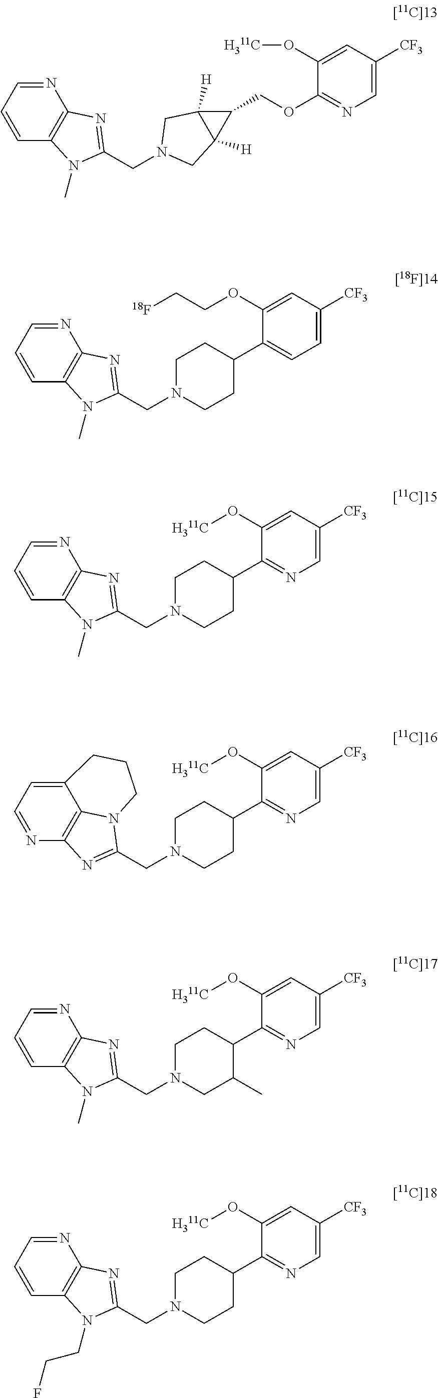 [SMILES: Cn1c(CN2C[C@@H]3[C@@H](COc4ncc(C(F)(F)F)cc4O[11CH3])[C@@H]3C2)nc2ncccc21 ; Cn1c(CN2CCC(c3ccc(C(F)(F)F)cc3OCC[18F])CC2)nc2ncccc21 ; Cn1c(CN2CCC(c3ncc(C(F)(F)F)cc3O[11CH3])CC2)nc2ncccc21 ; [11CH3]Oc1cc(C(F)(F)F)cnc1C1CCN(Cc2nc3nccc4c3n2CCC4)CC1 ; CC1CN(Cc2nc3ncccc3n2C)CCC1c1ncc(C(F)(F)F)cc1O[11CH3] ; [11CH3]Oc1cc(C(F)(F)F)cnc1C1CCN(Cc2nc3ncccc3n2CCF)CC1]

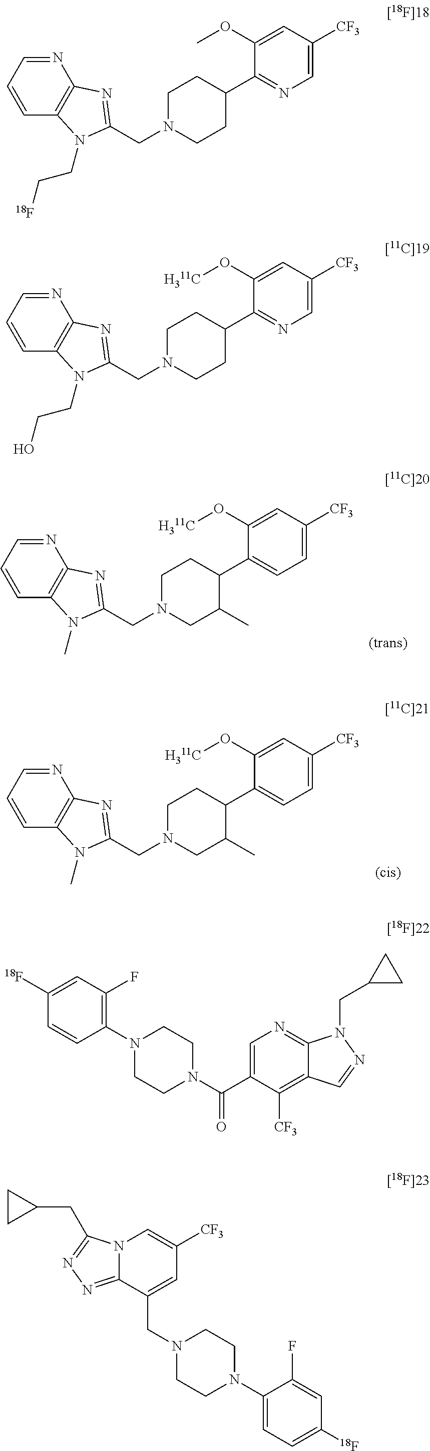 [SMILES: COc1cc(C(F)(F)F)cnc1C1CCN(Cc2nc3ncccc3n2CC[18F])CC1 ; [11CH3]Oc1cc(C(F)(F)F)cnc1C1CCN(Cc2nc3ncccc3n2CCO)CC1 ; CC1CN(Cc2nc3ncccc3n2C)CCC1c1ccc(C(F)(F)F)cc1O[11CH3] ; CC1CN(Cc2nc3ncccc3n2C)CCC1c1ccc(C(F)(F)F)cc1O[11CH3] ; O=C(c1cnc2c(cnn2CC2CC2)c1C(F)(F)F)N1CCN(c2ccc([18F])cc2F)CC1 ; Fc1cc([18F])ccc1N1CCN(Cc2cc(C(F)(F)F)cn3c(CC4CC4)nnc23)CC1]

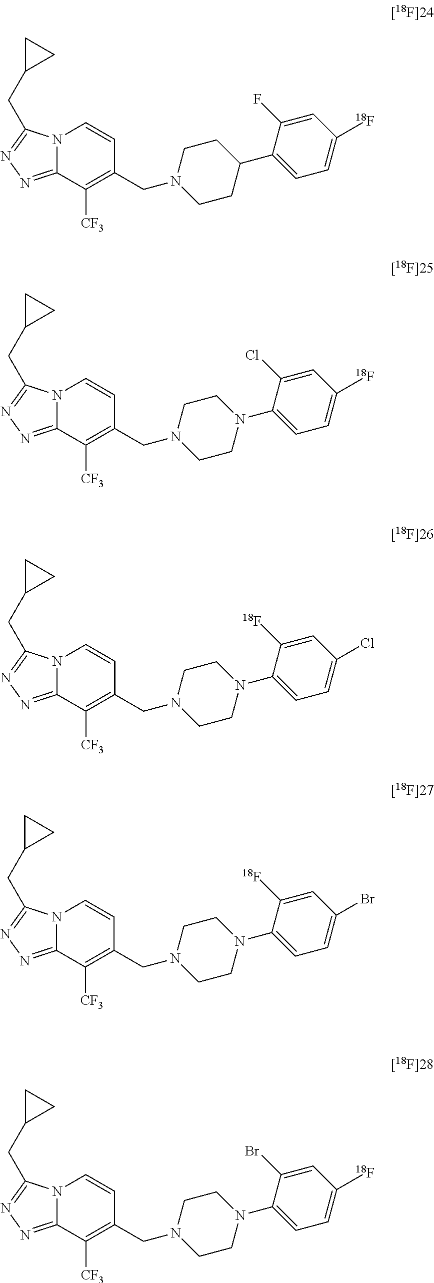 [SMILES: Fc1cc([18F])ccc1C1CCN(Cc2ccn3c(CC4CC4)nnc3c2C(F)(F)F)CC1 ; FC(F)(F)c1c(CN2CCN(c3ccc([18F])cc3Cl)CC2)ccn2c(CC3CC3)nnc12 ; FC(F)(F)c1c(CN2CCN(c3ccc(Cl)cc3[18F])CC2)ccn2c(CC3CC3)nnc12 ; FC(F)(F)c1c(CN2CCN(c3ccc(Br)cc3[18F])CC2)ccn2c(CC3CC3)nnc12 ; FC(F)(F)c1c(CN2CCN(c3ccc([18F])cc3Br)CC2)ccn2c(CC3CC3)nnc12]

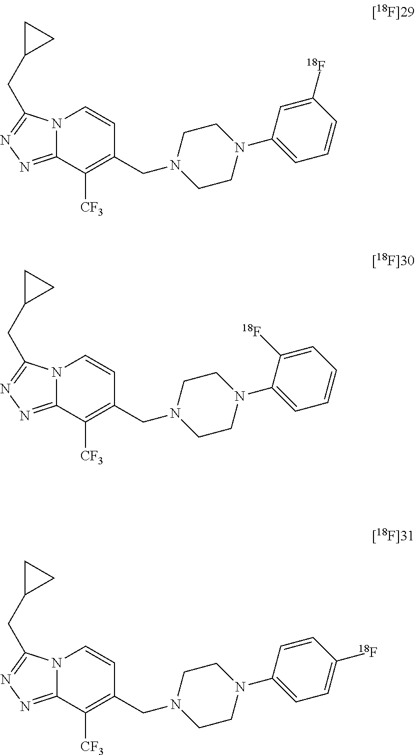 [SMILES: FC(F)(F)c1c(CN2CCN(c3cccc([18F])c3)CC2)ccn2c(CC3CC3)nnc12 ; FC(F)(F)c1c(CN2CCN(c3ccccc3[18F])CC2)ccn2c(CC3CC3)nnc12 ; FC(F)(F)c1c(CN2CCN(c3ccc([18F])cc3)CC2)ccn2c(CC3CC3)nnc12]

or a pharmaceutically acceptable salt thereof.

In some embodiments, the present disclosure provides a pharmaceutical composition comprising a compound of Formula (I), or a pharmaceutically acceptable salt thereof, and a pharmaceutically acceptable carrier.

In some embodiments, the present disclosure provides a method of imaging a brain of a subject, the method comprising:

i) administering to the subject an effective amount of a compound of Formula (I), or a pharmaceutically acceptable salt thereof;
ii) waiting a time sufficient to allow the compound to accumulate in the brain to be imaged; and
iii) imaging the brain with an imaging technique.

In some embodiments, the present disclosure provides a method of monitoring treatment of a psychiatric or a neurological disorder associated with mGluR2 in a subject, the method comprising:

i) administering to the subject an effective amount of a compound of Formula (I), or a pharmaceutically acceptable salt thereof;
ii) waiting a time sufficient to allow the compound of Formula (I) to accumulate in a brain of the subject;
iii) imaging the brain of the subject with an imaging technique;
iv) administering to the subject a therapeutic agent in an effective amount to treat the psychiatric or the neurological disorder;
v) after iv), administering to the subject an effective amount of a compound of Formula (I), or a pharmaceutically acceptable salt thereof;
vi) waiting a time sufficient to allow the compound of Formula (I) to accumulate in the brain of the subject;
vii) imaging the brain of the subject with an imaging technique; and
viii) comparing the image of step iii) and the image of step vii).

In some embodiments, the imaging technique is selected from positron emission tomography (PET) imaging, positron emission tomography with computer tomography (PET/CT) imaging, and positron emission tomography with magnetic resonance (PET/MRI) imaging.

In some embodiments, the neurological disorder associated with mGluR2 is selected from Alzheimer's disease, Parkinson's disease, dyskinesia, Lewy body disease, Prion disease, motor neuron disease (MND), and Fluntington's disease.

In some embodiments, the psychiatric disorder associated with mGluR2 is selected from schizophrenia, psychosis, anxiety, depression, drug abuse, pain, smoking cessation, and epilepsy.

In some embodiments, the present disclosure provides a compound of Formula (II)

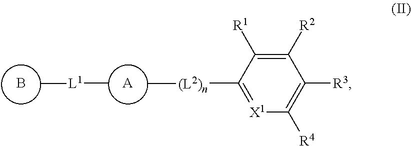

or a pharmaceutically acceptable salt thereof, wherein:
ring B is selected from formula (i), formula (ii), formula (v), and formula (vi):

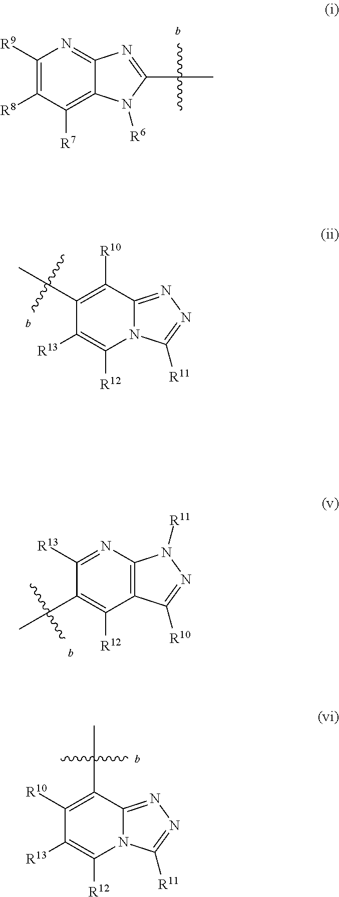

wherein b indicates a point of attachment of ring B to L¹;
L¹ is C₁₋₃ alkylene, which is optionally substituted with 1 or 2 substituents independently selected from halo, C₁₋₃ haloalkyl, OH, C₁₋₃ alkoxy, C₁₋₃ haloalkoxy, amino, C₁₋₆alkylamino, di(C₁₋₆ alkyl)amino, thio, and C₁₋₆alkylthio;
ring A is selected from formula (iii) and formula (iv):

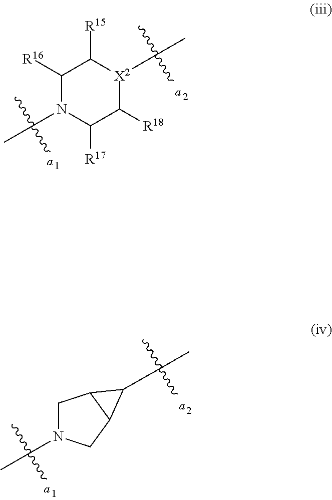

wherein a₁ indicates a point of attachment of ring A to L¹, and a₂ indicates a point of attachment of ring A to L²;
each L² is independently selected from C₁₋₃ alkylene, O, N(R^{N}), and S(═O)₂, wherein said C₁₋₃ alkylene is optionally substituted with 1 or 2 substituents independently selected from halo, C₁₋₃ haloalkyl, OH, C₁₋₃ alkoxy, C₁₋₃ haloalkoxy, amino, C₁₋₆alkylamino, di(C₁₋₆alkyl)amino, thio, and C₁₋₆ alkylthio;
n is 0, 1, 2, or 3;
each R^{N} is selected from H and C₁₋₃ alkyl;
X¹ is selected from N and CR⁵;
X² is selected from N and CR¹⁴;
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆alkylamino, di(C₁₋₆ alkyl)amino, thio, and C₁₋₆ alkylthio; and or R⁶ and R⁷, together with the carbon atom to which R⁷ is attached and the N atom to which R⁶ is attached form a 5-7-membered heterocycloalkyl ring, which is optionally substituted with 1 or 2 substituents independently selected from OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆alkylamino, di(C₁₋₆alkyl)amino, thio, and C₁₋₆ alkylthio;
provided that:
(a) if the ring B has formula (i) and X² is CR¹⁴, then X¹ is N or R¹ is C₁₋₃ haloalkoxy; and
(b) if the ring B has formula (ii), then X¹ is N.

In some embodiments of Formula (II):

L¹ is C₁₋₃ alkylene;
each L² is independently selected from C₁₋₃ alkylene, O, and N(R^{N});
n is 0, 1, or 2; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, halo, C₁₋₃ alkyl, C₁₋₃ alkoxy, C₁₋₃ haloalkyl, C₁₋₃ haloalkoxy, and C₃₋₁₀ cycloalkyl-C₁₋₃ alkyl.

In some embodiments, the compound of Formula (II) has formula:

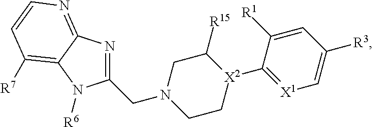

or a pharmaceutically acceptable salt thereof, wherein:
R¹⁵ is selected from H and C₁₋₃ alkyl;
X¹ is selected from N and CH;
X² is selected from N and CH;
R¹ and R³ are each independently selected from halo, C₁₋₃alkoxy, C₁₋₃ haloalkyl, and C₁₋₃ haloalkoxy;
R⁶ is C₁₋₃ alkyl, C₁₋₃ haloalkyl, or HO—C₁₋₃ alkyl; and
R⁷ is H, or R⁷ and R⁶ together with the atoms to which they are attached form a 6-membered heterocycloalkyl ring.

In some embodiments, the compound of Formula (II) is selected from:

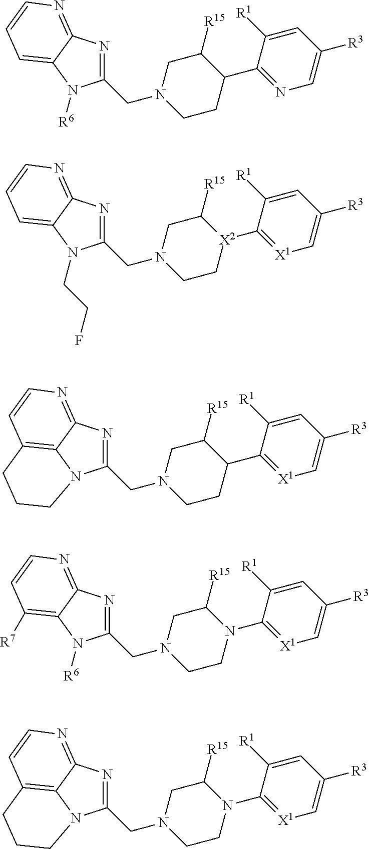

or a pharmaceutically acceptable salt thereof.

In some embodiments, the compound of Formula (II) has formula:

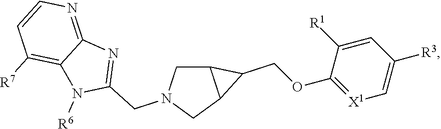

or a pharmaceutically acceptable salt thereof, wherein:
X¹ is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl; and
R⁷ is H, or R⁷ and R⁶ together with the atoms to which they are attached form a 6-membered heterocycloalkyl ring.

In some embodiments, the compound of Formula (II) is selected from:

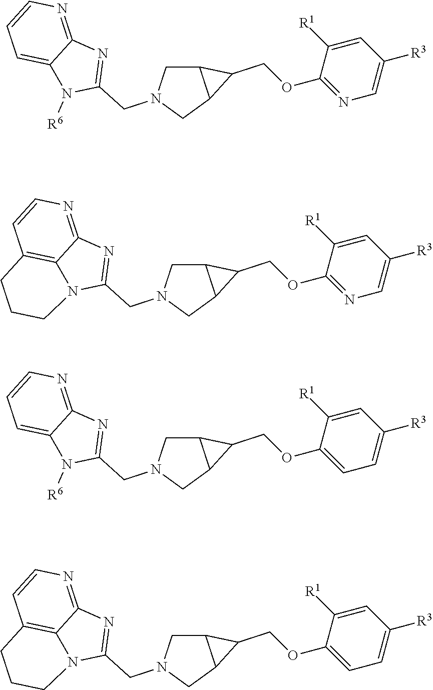

or a pharmaceutically acceptable salt thereof.

In some embodiments, the compound of Formula (II) has formula:

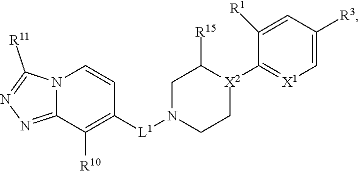

or a pharmaceutically acceptable salt thereof, wherein:
L¹ is selected from CH₂ and C(═O);
R¹¹ is C₃₋₁₀ cycloalkyl-C₁₋₃ alkyl;
R¹⁰ is selected from halo and C₁₋₃ haloalkyl;
R¹⁵ is selected from H and C₁₋₃ alkyl;
X² is selected from N and CH; and
R¹ and R³ are each independently selected from halo, C₁₋₃ alkoxy, C₁₋₃ haloalkyl, and C₁₋₃ haloalkoxy.

In some embodiments, the compound of Formula (II) is selected from any one of the following compounds:

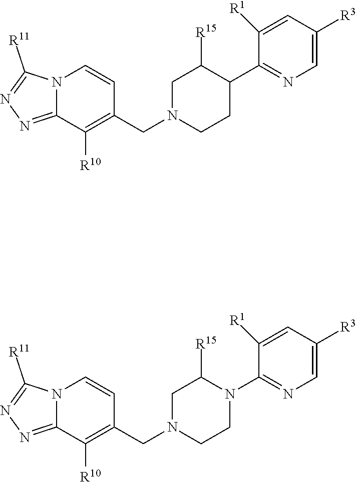

or a pharmaceutically acceptable salt thereof.

In some embodiments, the compound of Formula (II) has formula:

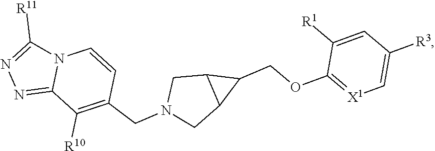

or a pharmaceutically acceptable salt thereof, wherein:
R¹¹ is C₃₋₁₀ cycloalkyl-C₁₋₃ alkyl;
R¹⁰ is selected from halo and C₁₋₃ haloalkyl;
X¹ is selected from N and CH; and
R¹ and R³ are each independently selected from halo, C₁₋₃ alkoxy, C₁₋₃ haloalkyl, and C₁₋₃ haloalkoxy.

In some embodiments, the compound of Formula (II) is selected from any one of the following compounds:

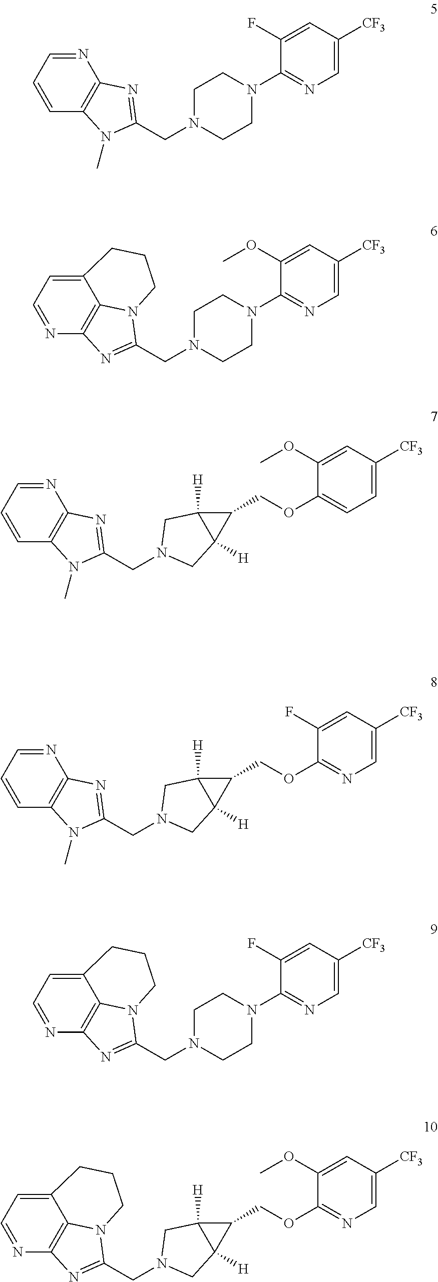 [SMILES: Cn1c(CN2CCN(c3ncc(C(F)(F)F)cc3F)CC2)nc2ncccc21 ; COc1cc(C(F)(F)F)cnc1N1CCN(Cc2nc3nccc4c3n2CCC4)CC1 ; COc1cc(C(F)(F)F)ccc1OC[C@@H]1[C@H]2CN(Cc3nc4ncccc4n3C)C[C@@H]12 ; Cn1c(CN2C[C@@H]3[C@@H](COc4ncc(C(F)(F)F)cc4F)[C@@H]3C2)nc2ncccc21 ; Fc1cc(C(F)(F)F)cnc1N1CCN(Cc2nc3nccc4c3n2CCC4)CC1 ; COc1cc(C(F)(F)F)cnc1OC[C@@H]1[C@H]2CN(Cc3nc4nccc5c4n3CCC5)C[C@@H]12]

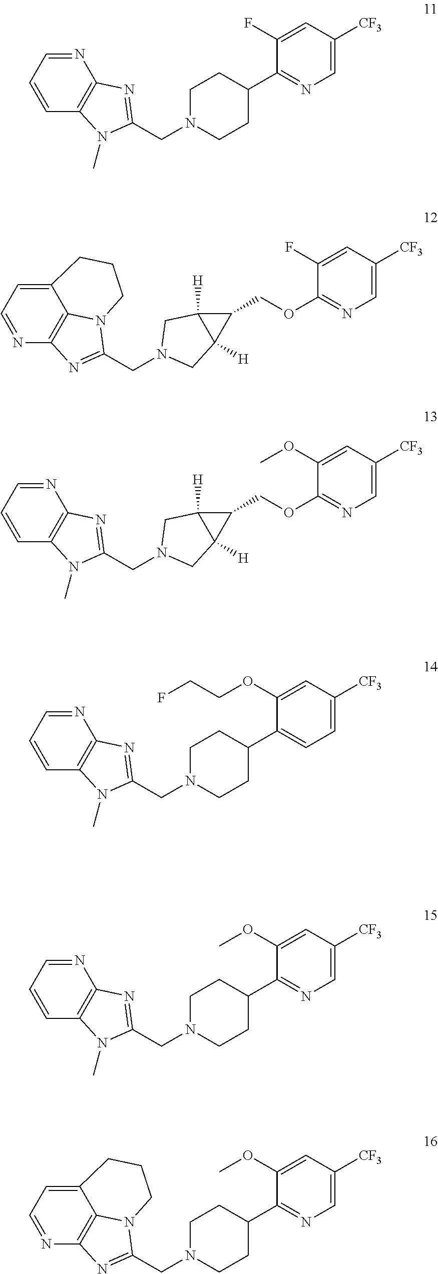 [SMILES: Cn1c(CN2CCC(c3ncc(C(F)(F)F)cc3F)CC2)nc2ncccc21 ; Fc1cc(C(F)(F)F)cnc1OC[C@@H]1[C@H]2CN(Cc3nc4nccc5c4n3CCC5)C[C@@H]12 ; COc1cc(C(F)(F)F)cnc1OC[C@@H]1[C@H]2CN(Cc3nc4ncccc4n3C)C[C@@H]12 ; Cn1c(CN2CCC(c3ccc(C(F)(F)F)cc3OCCF)CC2)nc2ncccc21 ; COc1cc(C(F)(F)F)cnc1C1CCN(Cc2nc3ncccc3n2C)CC1 ; COc1cc(C(F)(F)F)cnc1C1CCN(Cc2nc3nccc4c3n2CCC4)CC1]

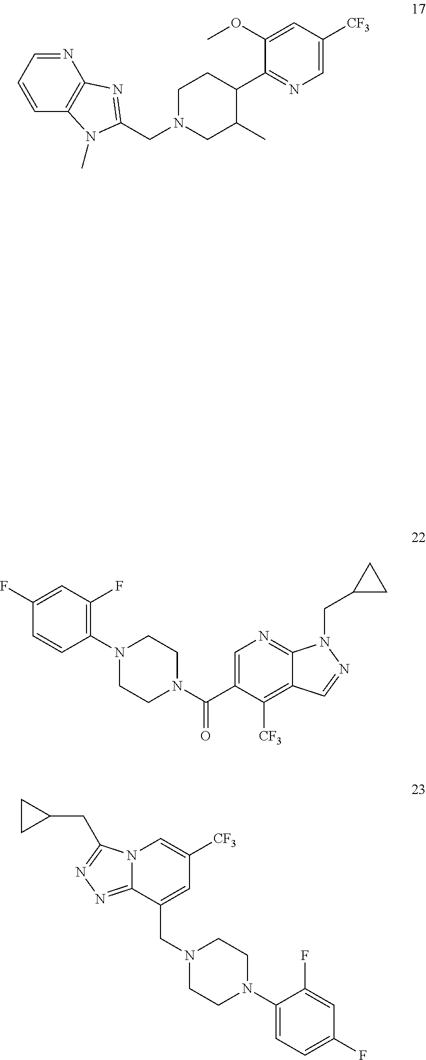 [SMILES: COc1cc(C(F)(F)F)cnc1C1CCN(Cc2nc3ncccc3n2C)CC1C ; O=C(c1cnc2c(cnn2CC2CC2)c1C(F)(F)F)N1CCN(c2ccc(F)cc2F)CC1 ; Fc1ccc(N2CCN(Cc3cc(C(F)(F)F)cn4c(CC5CC5)nnc34)CC2)c(F)c1]

or a pharmaceutically acceptable salt thereof.

In some embodiments, the present disclosure provides a pharmaceutical composition comprising a compound of Formula (II), or a pharmaceutically acceptable salt thereof, and a pharmaceutically acceptable carrier.

In some embodiments, the present disclosure provides a method of treating a psychiatric or a neurological disorder associated with mGluR2 in a subject, the method comprising administering to the subject in need thereof a therapeutically effective amount of a compound of Formula (II), or a pharmaceutically acceptable salt thereof.

In some embodiments, the neurological disorder associated with mGluR2 is selected from Alzheimer's disease, Parkinson's disease, dyskinesia, Lewy body disease, Prion disease, motor neuron disease (MND), and Huntington's disease.

In some embodiments, the psychiatric disorder is selected from schizophrenia, psychosis, anxiety, depression, drug abuse, pain, smoking cessation, and epilepsy.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains assay results showing mGluR2 PAM activity for the exemplary compounds.

FIG. 2 contains chemical structures of exemplary PET tracers for mGluR2.

FIG. 3 contains a scheme showing chemical synthesis of compounds 1, 2, and 7. Reagents and conditions: (a) Et₃N, MgSO₄, then Na(OAc)₃BH, DCE, rt, overnight; (b) PPh₃, diethyl azodicarboxylate solution (40 wt. % in toluene), THF, rt, 16 h; (c) TFA, DCM, rt, 2 h.

FIG. 4 contains a table showing the linear regression of the Log P of the reference compounds against the log of capacity factors k determined by HPLC.

FIG. 5 contains a table showing the Log P of 1, 2, and 7 determined by HPLC assay.

FIG. 6 contains a table showing plasma protein binding results of 1 and 2.

FIG. 7 contains a table showing plasma stability results for compounds 1 and 2.

FIG. 8 contains a table showing microsomal stability data for compounds 1 and 2, the natural logarithm (In) of peak area ratio data.

FIG. 9 contains a table showing microsomal stability results for compounds 1 and 2.

FIG. 10 contains a table showing the solution stability of compound 1 at the different pH.

FIG. 11 contains the target sequence of mGluR2 having 872 residues used for building the model for docking studies.

FIG. 12 contains Z-Scores for the hybrid model generated on YASARA for mGluR2 protein. The figure shows the initial model and all hybridized parts.

FIG. 13 contains (i) an image that was generated by ModFOLD based on residue accuracy prediction for the model or mGluR2 (upper image); and (ii) an image generated by QMEAN showing the local quality of the model for mGluR2 (lower image).

FIG. 14 contains a Ramachandran plot for the hybrid model built by YASARA for mGluR2. Plot generated with the SAVES server.

FIG. 15 contains a table showing docking scores for the known mGluR2 ligands into the designated binding site of the mGluR2 protein.

FIG. 16 contains an image of mGluR2 protein showing a position of the ligand binding site.

FIG. 17 contains an image showing results of the functional assays of mGluR1-6 and 8.

FIG. 18 contains snapshots of the docking results for compounds 1 (18a), 2 (18b) and 7 (18c). Pictures were rendered in PyMol 2.3.3. The interacting residues are shown in teal. Solid lines represent H-bonds, dotted lines show TI-TI stacking or indicate n-cation interaction.

FIG. 19 contains a line plot showing the mGluR2 PAM activity of compounds 1, 2, and 7.

FIG. 20 contains a table showing pharmacological properties of compounds 1, 2, and 7, and a table showing the in vitro stability characterization of compounds 1, 2, and 7.

FIG. 21A contains a bar graph showing assessment of BBB permeability for compounds 1, 2, and 7 via PAMPA assay. Pictures were rendered from Prism 5.0.

FIG. 21B contains a bar graph showing assessment of BBB permeability for compounds 1, 2, and 7 via Pgp-Glo assay. Pictures were rendered from Prism 5.0.

FIG. 22 contains a bar graph showing the ex vivo biodistribution in rat at four different time points post-[¹¹C]1 injection. Picture was rendered from Prism 5.0.

FIG. 23 contains images showing accumulation of [¹¹C]1 in different areas of rat brain at the time interval 10-15 min. Coronal level 1 shows uptake in the cingulate and motor cortex; level 2 in the striatum, level 3 in the thalamus and striatum, level 4 in the thalamus and hippocampus and level 5 in the cerebellum. Axial and sagittal views show activity distribution in the midbrain level. Slice thickness is 1.25 mm.

FIG. 24 contains a line plot showing in vivo binding profile of [¹¹C]1 in the rat brain, including that time-activity distribution of [¹¹C]1 in different brain areas show fast accumulation and reversible binding. The data is averaged of six normal Sprague Dawley rats.

FIG. 25 contains bar graphs showing in vivo binding profile of [¹¹C]1 in the rat brain, the blocking effect was calculated in the time interval 10-30 min after administration of [¹¹C]1. Cort=cortex, Str=striatum, Hippocamp=hippocampus, Thai=thalamus, Cereb=cerebellum and WB=whole brain. Pictures were rendered from Prism 5.0.

### DETAILED DESCRIPTION

The metabotropic glutamate receptor 2 (mGluR2) is widely expressed in the nervous systems [see refs. 1, 2]. mGluR2 expression is abundant in brain areas such as prefrontal cortex, hippocampus, amygdala, striatum, thalamus, cerebellum, and nucleus accumbens [see refs. 3, 4]. It predominantly localizes on presynaptic nerve terminals and modulates synaptic transmission and neuroplasticity [see refs. 3]. Structurally, mGluR2 has a characteristic extracellular Venus flytrap domain (VFTD), a seven transmembrane (7-TM) domain and a cysteine rich domain (CRD) that connects the mGluR dimers [see refs. 5]. The therapeutic benefits of mGluR2 modulators have been shown for a variety of conditions including Alzheimer's disease [see refs. 6-9], schizophrenia [see refs. 10-13], depression [see refs. 14], anxiety [see refs. 15] and pain [see refs. 16-18].

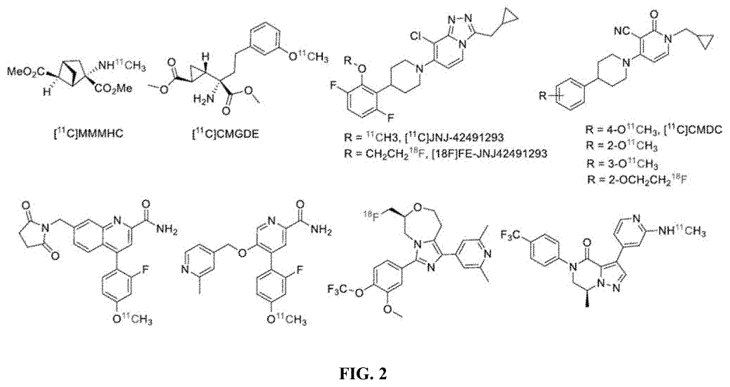

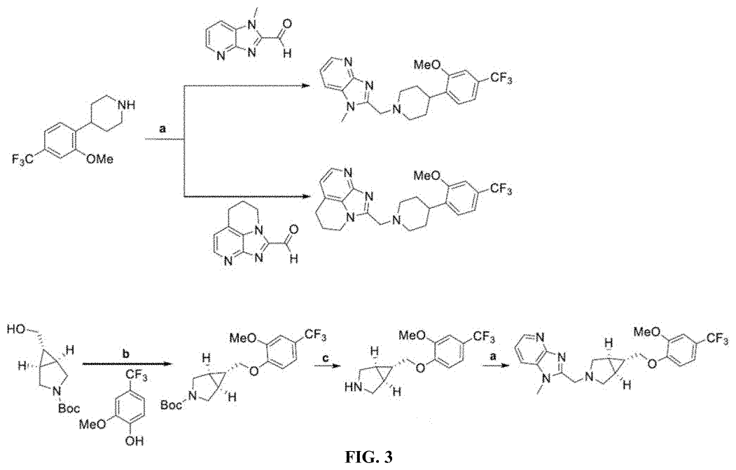

Several PET radiotracers for mGluR2 have been derived from allosteric modulators that target the 7-TM instead of VFTD region of mGluR2. It is believed that the allosteric modulators would bear higher lipophilicity and mGluR2 selectivity than orthosteric ligands due to the hydrophobicity and heterogeneity of the 7-TM binding pocket across mGlu receptors [see refs. 21-23]. So far, two radioligands in this category have been advanced for human clinical trials, including mGluR2 PAM [¹¹C]JNJ42491293 (FIG. 2) and a radioligand from Merck. Flowever, [¹¹C]JNJ42491293 (FIG. 2) was not found useful for the visualization and quantification of mGluR2 in vivo because of its apparent off-target binding [see refs. 24, 25]. The Merck radiotracer was only reported in an abstract without information on its chemical structure and detailed imaging results [see refs. 26, 27]. The fluorine-18 labeled derivative, [¹⁸F]FE-JNJ-42491293 (see FIG. 2), was disclosed in an abstract but it is not clear if this tracer has the similar off-target binding as its ¹¹C counterpart [see ref. 28]. Other mGluR2 PAM tracers (see FIG. 2) exhibited an insufficient affinity and low BBB-penetration. PET imaging with these compounds did not enable in vivo visualization of the living rat brain [see refs. 29, 30]. The mGluR2 NAM tracers, on the other hand, showed off-target binding and limited brain uptake with intensive interaction with brain efflux pumps on the murine BBB.

In contrast, the mGluR2 PAM benzimidazole derivatives within the present claims are efficient and efficacious mGluR2 PET tracers, as described herein, compounds 1 (EC₅₀=13 nM) [see ref. 37] and 2 (EC₅₀=5 nM) [see ref. 36]. The presence of 2-methoxy-4-trifluoromethylphenyl group in compounds 1 and 2 allows rapid radiolabeling of the phenol precursor via O-[¹¹C]methylation with [¹¹C]CH₃I. Compound 7 was further designed as a PET imaging candidate [see ref. 38]. The structurally distinct compound 3, a potent and selective mGluR2 PAM (EC₅₀=78 nM), was used as a selective blocking reagent during the investigation of [¹¹C]JNJ42491293 and therefore it was used as a blocking reagent in the present studies [see ref. 30]. Described herein are the design, synthesis and characterization of mGluR2 PAM-active compounds using in silico modeling, in vitro assays and in vivo PET imaging methods to evaluate their potential as mGluR2-specific PET imaging ligands and therapeutics. Pharmaceutical compositions, as well as method of making and using these compound for treating various psychiatric and neurological diseases and conditions are also described.

### Therapeutic Compounds

In some embodiments, the present disclosure provides a compound of Formula:

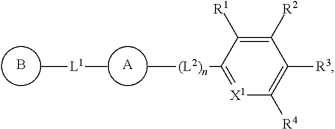

or a pharmaceutically acceptable salt thereof, wherein ring B, L¹, ring A, L², n, X¹, R¹, R², R³, and R⁴ are described herein (e.g., for Formula (I) or Formula (II)). In some embodiments, the compound of the above Formula has Formula (I) when it comprises at least one radioisotope selected from ¹¹C and ¹⁸F. In some embodiments, the compound of the above Formula has Formula (II) when it comprises only stable isotopes (i.e., the compound of Formula (II) does not comprise any radioisotopes).

Certain embodiments of ring B, L¹, ring A, L², n, X¹, R¹, R², R³, and R⁴ are described as follows (in Formula (I) or Formula (II)). In some embodiments:
ring B is selected from formula (i), formula (ii), formula (v), and formula (vi):

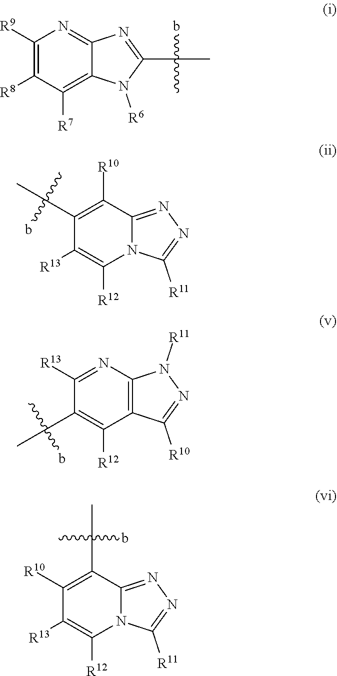

wherein b indicates a point of attachment of ring B to L¹;
L¹ is C₁₋₃ alkylene, which is optionally substituted with 1 or 2 substituents independently selected from halo, C₁₋₃ haloalkyl, OH, C₁₋₃ alkoxy, C₁₋₃ haloalkoxy, amino, C₁₋₆alkylamino, di(C₁₋₆ alkyl)amino, thio, and C₁₋₆ alkylthio;
ring A is selected from formula (Hi) and formula (iv):

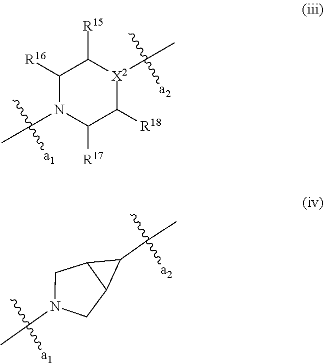

wherein a₁ indicates a point of attachment of ring A to L¹, and a₂ indicates a point of attachment of ring A to L²;
each L² is independently selected from C₁₋₃ alkylene, O, N(R^{N}), and S(═O)₂, wherein said C₁₋₃ alkylene is optionally substituted with 1 or 2 substituents independently selected from halo, C₁₋₃ haloalkyl, OH, C₁₋₃ alkoxy, C₁₋₃ haloalkoxy, amino, C₁₋₆alkylamino, di(C₁₋₆alkyl)amino, thio, and C₁₋₆ alkylthio;
each R^{N} is selected from H and C₁₋₃ alkyl;
n is 0, 1, 2, or 3;
X¹ is selected from N and CR⁵;
X² is selected from N and CR¹⁴; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆alkylamino, di(C₁₋₆ alkyl)amino, thio, and C₁₋₆ alkylthio;
or R⁶ and R⁷, together with the carbon atom to which R⁷ is attached and the N atom to which R⁶ is attached form a 5-7-membered heterocycloalkyl ring, which is optionally substituted with 1 or 2 substituents independently selected from OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆alkylamino, di(C₁₋₆alkyl)amino, thio, and C₁₋₆ alkylthio.

In some embodiments of Formula (I), the compound comprises at least one radioisotope selected from ¹¹C and ¹⁸F. In some embodiments of Formula (I), the compound comprises at least one ¹¹C radioisotope. In some embodiments of Formula (I), the compound comprises at least one ¹⁸F radioisotope.

In some embodiments, the compound of Formula (I) is not:

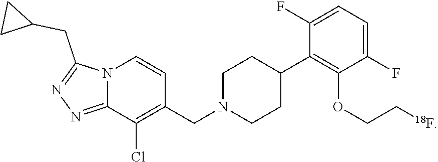

In some embodiments, the compound of Formula (I) is not:

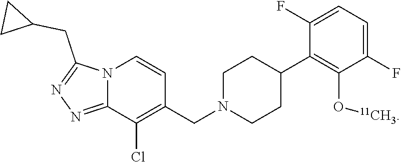

In some embodiments of the compound of Formula (II), if the ring B has formula (i) and X² is CR¹⁴, then X¹ is N or R¹ is C₁₋₃ haloalkyl. In some embodiments of Formula (II), if the ring B has formula (ii), then X¹ is N.

In some embodiments, the compound of Formula (II) is not any one of the following compounds:

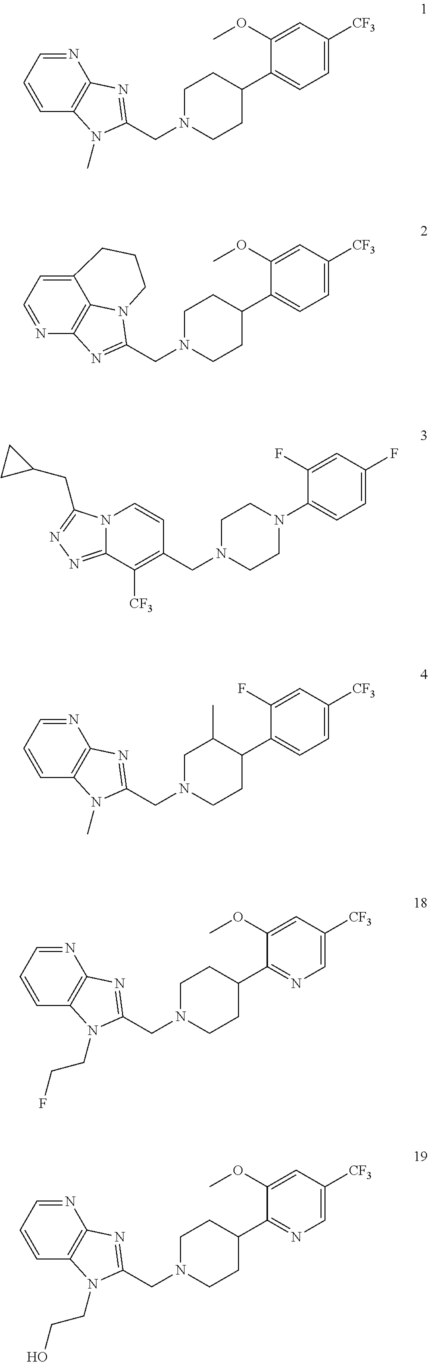 [SMILES: COc1cc(C(F)(F)F)ccc1C1CCN(Cc2nc3ncccc3n2C)CC1 ; COc1cc(C(F)(F)F)ccc1C1CCN(Cc2nc3nccc4c3n2CCC4)CC1 ; Fc1ccc(N2CCN(Cc3ccn4c(CC5CC5)nnc4c3C(F)(F)F)CC2)c(F)c1 ; CC1CN(Cc2nc3ncccc3n2C)CCC1c1ccc(C(F)(F)F)cc1F ; COc1cc(C(F)(F)F)cnc1C1CCN(Cc2nc3ncccc3n2CCF)CC1 ; COc1cc(C(F)(F)F)cnc1C1CCN(Cc2nc3ncccc3n2CCO)CC1]

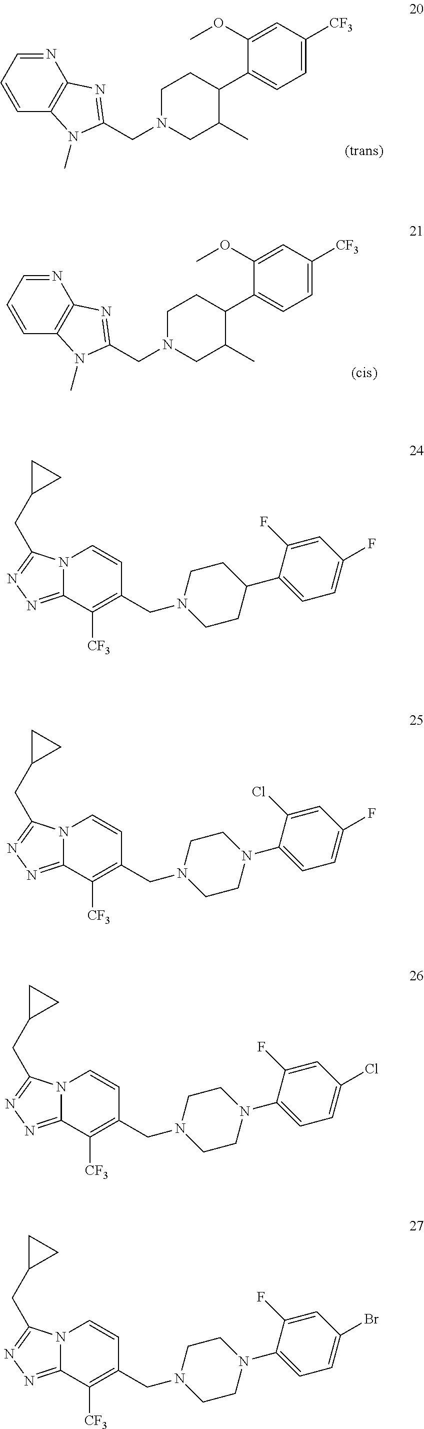 [SMILES: COc1cc(C(F)(F)F)ccc1C1CCN(Cc2nc3ncccc3n2C)CC1C ; COc1cc(C(F)(F)F)ccc1C1CCN(Cc2nc3ncccc3n2C)CC1C ; Fc1ccc(C2CCN(Cc3ccn4c(CC5CC5)nnc4c3C(F)(F)F)CC2)c(F)c1 ; Fc1ccc(N2CCN(Cc3ccn4c(CC5CC5)nnc4c3C(F)(F)F)CC2)c(Cl)c1 ; Fc1cc(Cl)ccc1N1CCN(Cc2ccn3c(CC4CC4)nnc3c2C(F)(F)F)CC1 ; Fc1cc(Br)ccc1N1CCN(Cc2ccn3c(CC4CC4)nnc3c2C(F)(F)F)CC1]

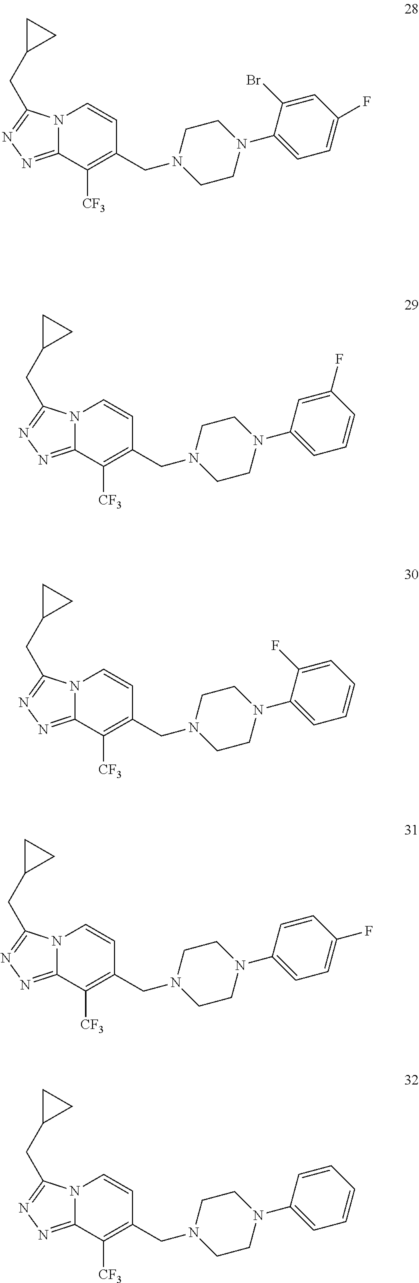 [SMILES: Fc1ccc(N2CCN(Cc3ccn4c(CC5CC5)nnc4c3C(F)(F)F)CC2)c(Br)c1 ; Fc1cccc(N2CCN(Cc3ccn4c(CC5CC5)nnc4c3C(F)(F)F)CC2)c1 ; Fc1ccccc1N1CCN(Cc2ccn3c(CC4CC4)nnc3c2C(F)(F)F)CC1 ; Fc1ccc(N2CCN(Cc3ccn4c(CC5CC5)nnc4c3C(F)(F)F)CC2)cc1 ; FC(F)(F)c1c(CN2CCN(c3ccccc3)CC2)ccn2c(CC3CC3)nnc12]

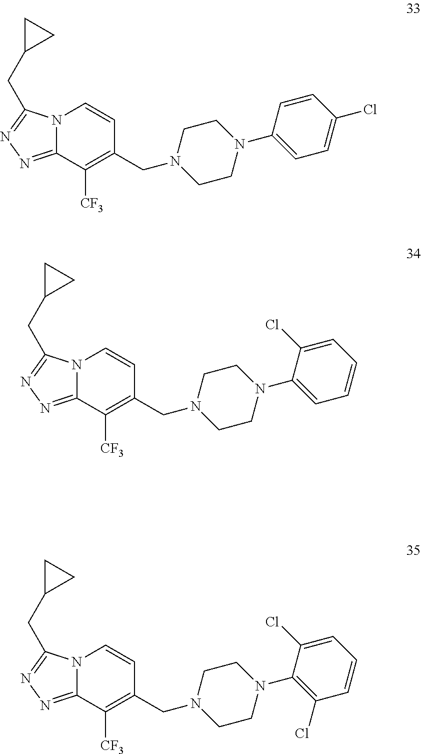 [SMILES: FC(F)(F)c1c(CN2CCN(c3ccc(Cl)cc3)CC2)ccn2c(CC3CC3)nnc12 ; FC(F)(F)c1c(CN2CCN(c3ccccc3Cl)CC2)ccn2c(CC3CC3)nnc12 ; FC(F)(F)c1c(CN2CCN(c3c(Cl)cccc3Cl)CC2)ccn2c(CC3CC3)nnc12]

In some embodiments, ring B has formula (i):

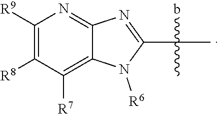

In some embodiments, ring B has formula (i):

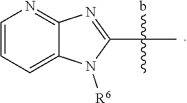

In some embodiments, ring B has formula (i):

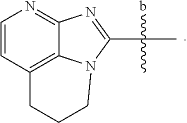

In some embodiments, ring B has formula (ii):

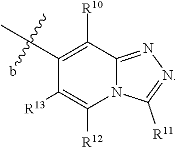

In some embodiments, ring B has formula (ii):

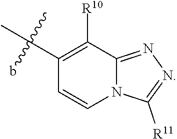

In some embodiments, ring B has formula (v):

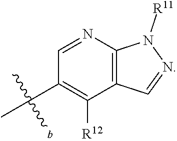

In some embodiments, ring B has formula (vi):

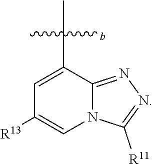

In some embodiments, L¹ is C₁₋₃ alkylene, which is optionally substituted with halo, C₁₋₃ haloalkyl, C₁₋₃ alkoxy, or C₁₋₃ haloalkoxy. In some embodiments, L¹ is C₁₋₃ alkylene (e.g., ethylene, methylene, or n-propylene).

In some embodiments, L¹ is C₁₋₃ alkylene, which is optionally substituted with oxo. In some embodiments, L¹ is C(═O).

In some embodiments, ring A has formula (iii):

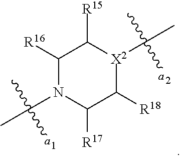

In some embodiments, ring A has formula (iii):

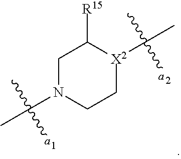

In some embodiments, ring A has formula (iii):

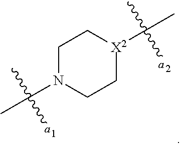

In some embodiments, ring A has formula (iii):

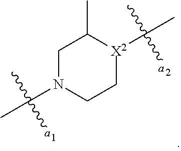

In some embodiments, ring A has formula (iv):

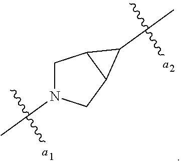 [SMILES: *C1C2CN(*)CC12]

In some embodiments, ring A has formula (iv):

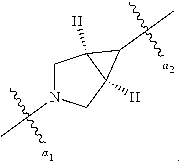 [SMILES: *C1[C@H]2CN(*)C[C@@H]12]

In some embodiments, ring B is formula (i) and ring A is formula (iii).

In some embodiments, ring B is formula (i) and ring A is formula (iv).

In some embodiments, ring B is formula (ii) and ring A is formula (iii).

In some embodiments, ring B is formula (ii) and ring A is formula (iv).

In some embodiments, ring B is formula (v) and ring A is formula (iii).

In some embodiments, ring B is formula (vi) and ring A is formula (iii).

In some embodiments, each L² is independently selected from C₁₋₃ alkylene, O, and N(R^{N}), wherein said C₁₋₃ alkylene is optionally substituted with halo, C₁₋₃ haloalkyl, C₁₋₃ alkoxy, or C₁₋₃ haloalkoxy. In some embodiments, each L² is independently selected from C₁₋₃ alkylene, O, and NH. In some embodiments, each L² is independently selected from C₁₋₃ alkylene and O.

In some embodiments, R^{N} is H. In some embodiments, R^{N} is C₂₋₃ alkyl.

In some embodiments, n is 0 (i.e., (L²)ₙ is a bond between ring A and the phenyl/pyridinyl ring). In some embodiments, n is 1 (e.g., (L²)ₙ is C₁₋₃ alkylene (e.g., methylene, ethylene, n-propylene). In some embodiments, n is 2. (e.g., (L²)ₙ is C₁₋₃ alkylene-O or C₁₋₃ alkylene-NH). In some embodiments, n is 3.

In some embodiments, X¹ is N. In some embodiments, X¹ is CR⁵. In some embodiments, X¹ is CH.

In some embodiments, X² is N. In some embodiments, X² is CR¹⁴. In some embodiments, X² is CH.

In some embodiments, X¹ is N, and X² is N.

In some embodiments, X¹ is N, and X² is CR¹⁴.

In some embodiments, X¹ is CR⁵, and X² is N.

In some embodiments, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, halo, C₁₋₃ alkyl, C₁₋₃ alkoxy, C₁₋₃ haloalkyl, C₁₋₃ haloalkoxy, and C₃₋₁₀ cycloalkyl-C₁₋₃ alkyl.

In some embodiments of formula (I), at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is halo, C₁₋₃ haloalkyl, or C₁₋₃ haloalkoxy comprising at least one radioisotope ¹⁸F.

In some embodiments of formula (I), at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ is C₁₋₃ alkoxy comprising at least one radioisotope ¹¹C.

In some embodiments of formula (I), at least one of R¹, R², R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ is selected from ¹⁸F, ¹¹CH₃O, CF₂¹⁸F, ¹⁸FCH₂O, ¹⁸FCD₂O, ¹⁸FCH₂CH₂O, and ¹⁸FCD₂CD₂O.

In some embodiments:

L¹ is C₁₋₃ alkylene;
each L² is independently selected from C₁₋₃ alkylene, O, and N(R^{N});
n is 0, 1, or 2; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, halo, C₁₋₃ alkyl, C₁₋₃ alkoxy, C₁₋₃ haloalkyl, C₁₋₃ haloalkoxy, and C₃₋₁₀ cycloalkyl-C₁₋₃ alkyl.

In some embodiments, R¹ and R³ are each independently selected from halo, C₁₋₃ alkoxy, C₁₋₃ haloalkyl, and C₁₋₃ haloalkoxy.

In some embodiments, R¹ is selected from C₁₋₃ alkoxy, halo, and C₁₋₃ haloalkoxy. In some embodiments of formula (I), R¹ is selected from ¹⁸F, ¹¹CH₃O, CF₂¹⁸F, ¹⁸FCH₂O, ¹⁸FCD₂O, ¹⁸FCH₂CH₂O, and ¹⁸FCD₂CD₂O.

In some embodiments, R² and R⁴ are each H.

In some embodiments, R³ is selected from C₁₋₃ haloalkyl and halo. In some embodiments of formula (I), R³ is selected from ¹⁸F, ¹¹CH₃O, CF₂¹⁸F, ¹⁸FCH₂O, ¹⁸FCD₂O, ¹⁸FCH₂CH₂O, and ¹⁸FCD₂CD₂O.

In some embodiments of formula (I), R³ is selected from ¹⁸F, and CF₂¹⁸F.

In some embodiments, R⁶ is C₁₋₃ alkyl (e.g. methyl, ethyl, n-propyl).

In some embodiments, R¹¹ is C₃₋₁₀ cycloalkyl-C₁₋₃ alkyl.

In some embodiments, R¹⁰ is selected from halo and C₁₋₃ haloalkyl. In some embodiments, R¹⁰ is halo. In some embodiments, R¹⁰ is C₁₋₃ haloalkyl.

In some embodiments, R¹⁵ is selected from H and C₁₋₃ alkyl. In some embodiments, R¹⁵ is C₁₋₃ alkyl. In some embodiments, the carbon atom to which R¹⁵ is attached is in 5 configuration. In some embodiments, the carbon atom to which R¹⁵ is attached is in R configuration.

In some embodiments, R², R⁴, R⁵, R⁷, R⁸, R⁹, R¹², R¹³, R¹⁴, R¹⁶, R¹⁷, and R¹⁸ are each H.

In some embodiments, the compound has formula:

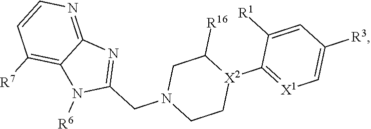

or a pharmaceutically acceptable salt thereof, wherein R⁶, R⁷, R¹, R³, X¹, X², and R¹⁵ are as described herein.

In some embodiments:
R¹⁵ is selected from H and C₁₋₃ alkyl;
X¹ is selected from N and CH;
X² is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl, C₁₋₃ haloalkyl, or HO—C₁₋₃ alkyl; and
   R⁷ is H, or R⁷ and R⁶ together with the atoms to which they are attached form a 6-membered heterocycloalkyl ring.

In some embodiments, R⁶ is C₁₋₃ alkyl.

In some embodiments, the compound has formula:

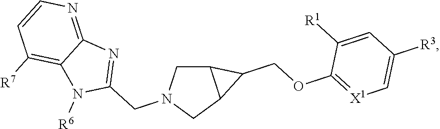

or a pharmaceutically acceptable salt thereof, wherein R⁶, R⁷, R¹, R³, and X¹ are as described herein.

In some embodiments:
X¹ is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl; and
   R⁷ is H, or R⁷ and R⁶ together with the atoms to which they are attached form a 6-membered heterocycloalkyl ring.

In some embodiments, the compound has formula:

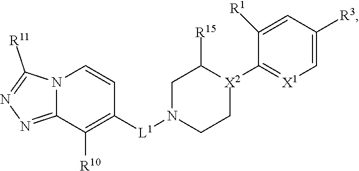

or a pharmaceutically acceptable salt thereof, wherein R¹, R³, X¹, X², R¹⁵, R¹⁰, and R¹¹ are as described herein.

In some embodiments:
L¹ is selected from CH₂ and C(═O);
R¹¹ is C₃₋₁₀ cycloalkyl-C₁₋₃ alkyl;
R¹⁰ is selected from halo and C₁₋₃ haloalkyl;
R¹⁵ is selected from H and C₁₋₃ alkyl;
X¹ is selected from N and CH;
X² is selected from N and CH; and
   R¹ and R³ are each independently selected from halo, C₁₋₃ alkoxy, C₁₋₃ haloalkyl, and C₁₋₃ haloalkoxy.

In some embodiments, the compound has formula:

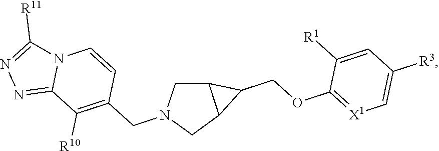

or a pharmaceutically acceptable salt thereof, wherein R¹, R³, X¹, R¹⁰, and R¹¹ are as described herein.

In some embodiments:

R¹¹ is C₃₋₁₀ cycloalkyl-C₁₋₃ alkyl;
R¹⁰ is selected from halo and C₁₋₃ haloalkyl;
X¹ is selected from N and CH; and
R¹ and R³ are each independently selected from halo, C₁₋₃alkoxy, C₁₋₃ haloalkyl, and C₁₋₃ haloalkoxy.

In some embodiments, the compound of Formula (I) is:

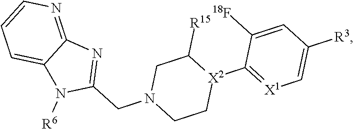

or a pharmaceutically acceptable salt thereof, wherein R³, R⁶, X¹, X², and R¹⁵ are as described herein.

In some embodiments, the compound of Formula (I) is:

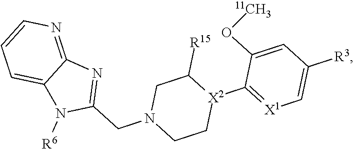

or a pharmaceutically acceptable salt thereof, wherein R³, R⁶, X¹, X², and R¹⁵ are as described herein.

In some embodiments, the compound of Formula (I) is:

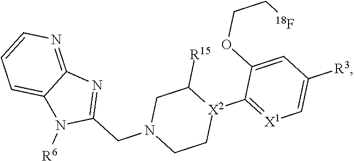

or a pharmaceutically acceptable salt thereof, wherein R³, R⁶, X¹, X², and R¹⁵ are as described herein.

In some embodiments, the compound of Formula (I) is:

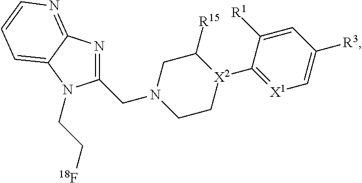

or a pharmaceutically acceptable salt thereof, wherein R³, R¹, X¹, X², and R¹⁵ are as described herein.

In some embodiments, the compound of Formula (I) is:

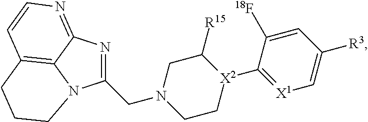

or a pharmaceutically acceptable salt thereof, wherein R³, X¹, X², and R¹⁵ are as described herein.

In some embodiments, the compound of Formula (I) is:

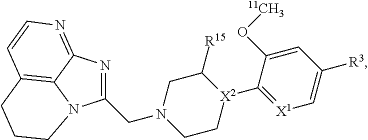

or a pharmaceutically acceptable salt thereof, wherein R³, X¹, X², and R¹⁵ are as described herein.

In some embodiments, the compound of Formula (I) is:

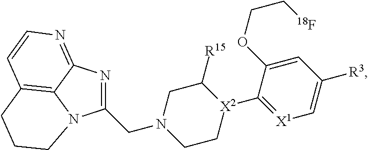

or a pharmaceutically acceptable salt thereof, wherein R³, X¹, X², and R¹⁵ are as described herein.

In some embodiments, the compound of Formula (I) is:

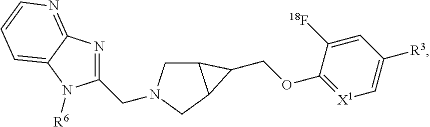

or a pharmaceutically acceptable salt thereof, wherein R³, X¹, and R⁶ are as described herein.

In some embodiments, the compound of Formula (I) is:

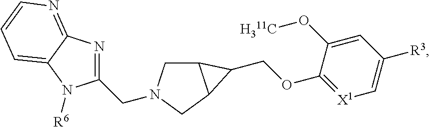

or a pharmaceutically acceptable salt thereof, wherein R³, X¹, and R⁶ are as described herein.

In some embodiments, the compound of Formula (I) is:

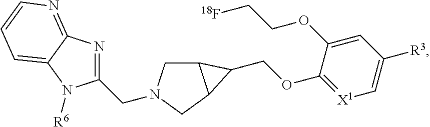

or a pharmaceutically acceptable salt thereof, wherein R³, X¹, and R⁶ are as described herein.

In some embodiments, the compound of Formula (I) is:

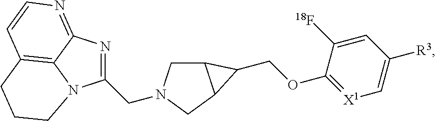

or a pharmaceutically acceptable salt thereof, wherein R³ and X¹ are as described herein.

In some embodiments, the compound of Formula (I) is:

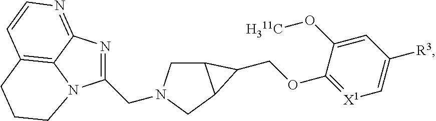

or a pharmaceutically acceptable salt thereof, wherein R³ and X¹ are as described herein.

In some embodiments, the compound of Formula (I) is:

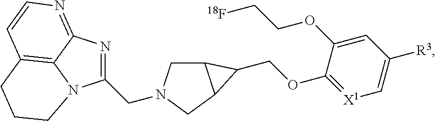

or a pharmaceutically acceptable salt thereof, wherein R³ and X¹ are as described herein.

In some embodiments, the compound of Formula (I) is:

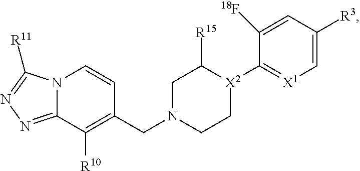

or a pharmaceutically acceptable salt thereof, wherein R³, X¹, X², R¹⁵, R¹⁰, and R¹¹ are as described herein.

In some embodiments, the compound of Formula (I) is:

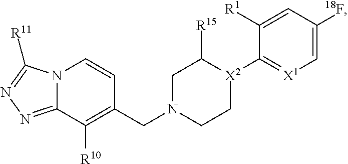

or a pharmaceutically acceptable salt thereof, wherein R¹, X¹, X², R¹⁵, R¹⁰, and R¹¹ are as described herein.

In some embodiments, the compound of Formula (I) is:

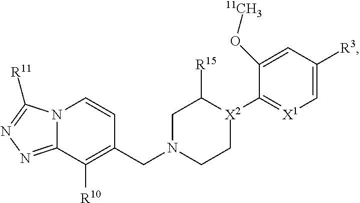

or a pharmaceutically acceptable salt thereof, wherein R³, X¹, X², R¹⁵, R¹⁰, and R¹¹ are as described herein.

In some embodiments, the compound of Formula (I) is:

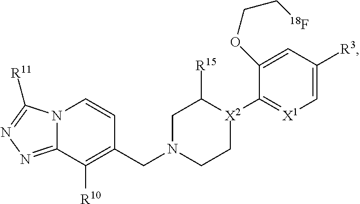

or a pharmaceutically acceptable salt thereof, wherein R³, X¹, X², R¹⁵, R¹⁰, and R¹¹ are as described herein.

In some embodiments, the compound of Formula (I) comprises at least one group selected from ¹⁸F, ¹¹CH₃O, CF₂¹⁸F, ¹⁸FCH₂O, ¹⁸FCD₂O, and ¹⁸FCH₂CH₂O.

In some embodiments, the compound of Formula (I) is selected from any one of the following compounds:

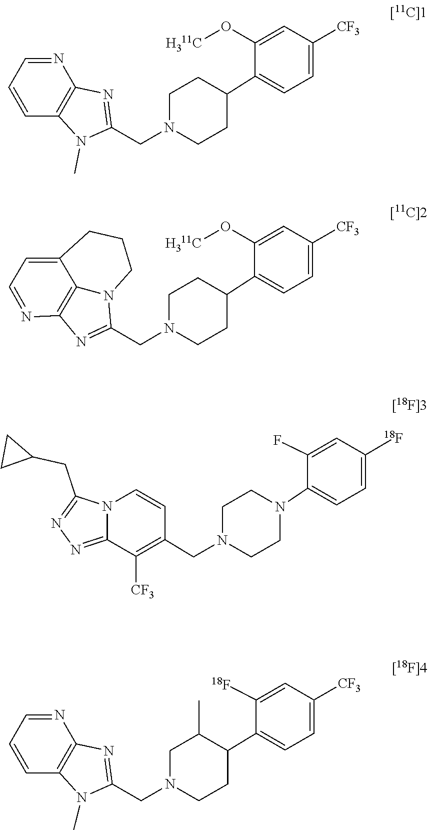 [SMILES: Cn1c(CN2CCC(c3ccc(C(F)(F)F)cc3O[11CH3])CC2)nc2ncccc21 ; [11CH3]Oc1cc(C(F)(F)F)ccc1C1CCN(Cc2nc3nccc4c3n2CCC4)CC1 ; Fc1cc([18F])ccc1N1CCN(Cc2ccn3c(CC4CC4)nnc3c2C(F)(F)F)CC1 ; CC1CN(Cc2nc3ncccc3n2C)CCC1c1ccc(C(F)(F)F)cc1[18F]]

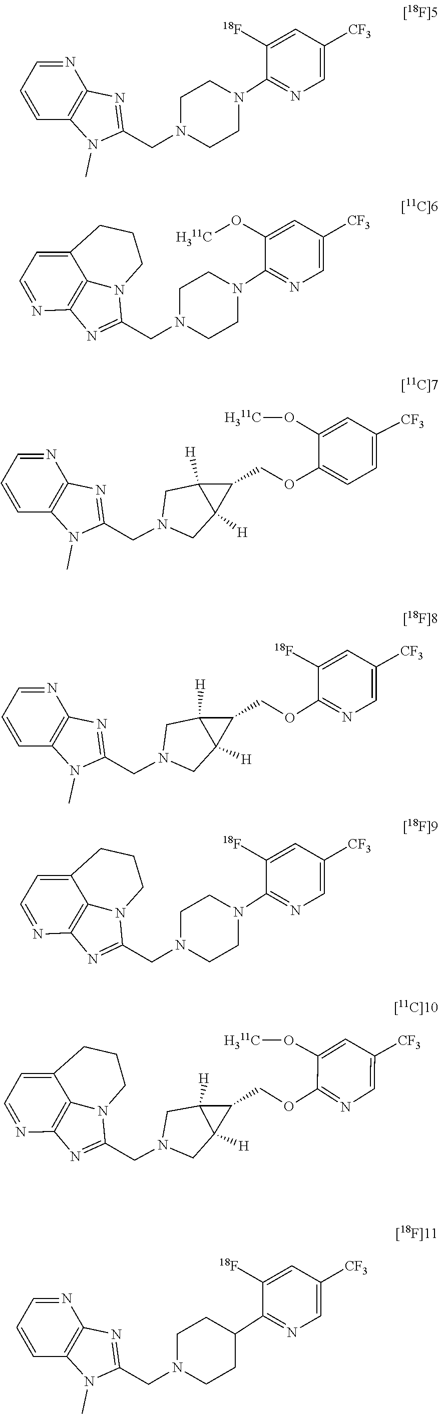

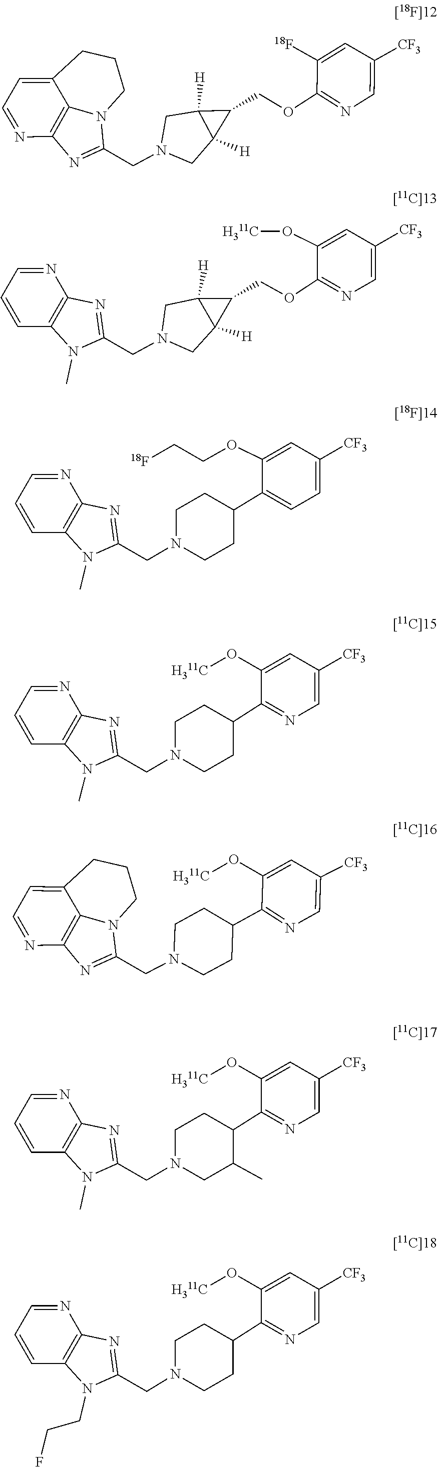

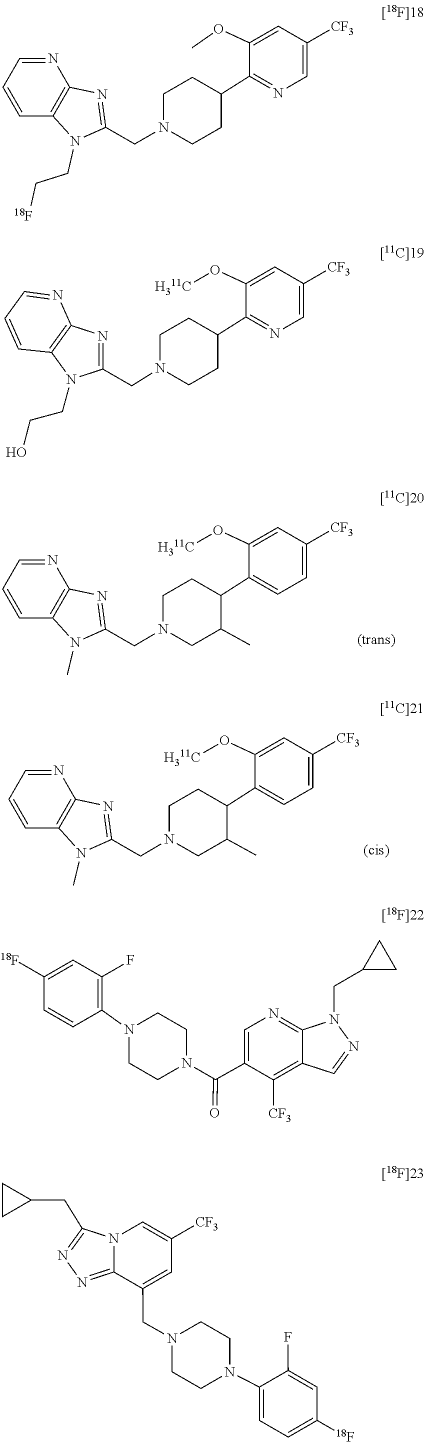 [SMILES: COc1cc(C(F)(F)F)cnc1C1CCN(Cc2nc3ncccc3n2CC[18F])CC1 ; [11CH3]Oc1cc(C(F)(F)F)cnc1C1CCN(Cc2nc3ncccc3n2CCO)CC1 ; CC1CN(Cc2nc3ncccc3n2C)CCC1c1ccc(C(F)(F)F)cc1O[11CH3] ; CC1CN(Cc2nc3ncccc3n2C)CCC1c1ccc(C(F)(F)F)cc1O[11CH3] ; O=C(c1cnc2c(cnn2CC2CC2)c1C(F)(F)F)N1CCN(c2ccc([18F])cc2F)CC1 ; Fc1cc([18F])ccc1N1CCN(Cc2cc(C(F)(F)F)cn3c(CC4CC4)nnc23)CC1]

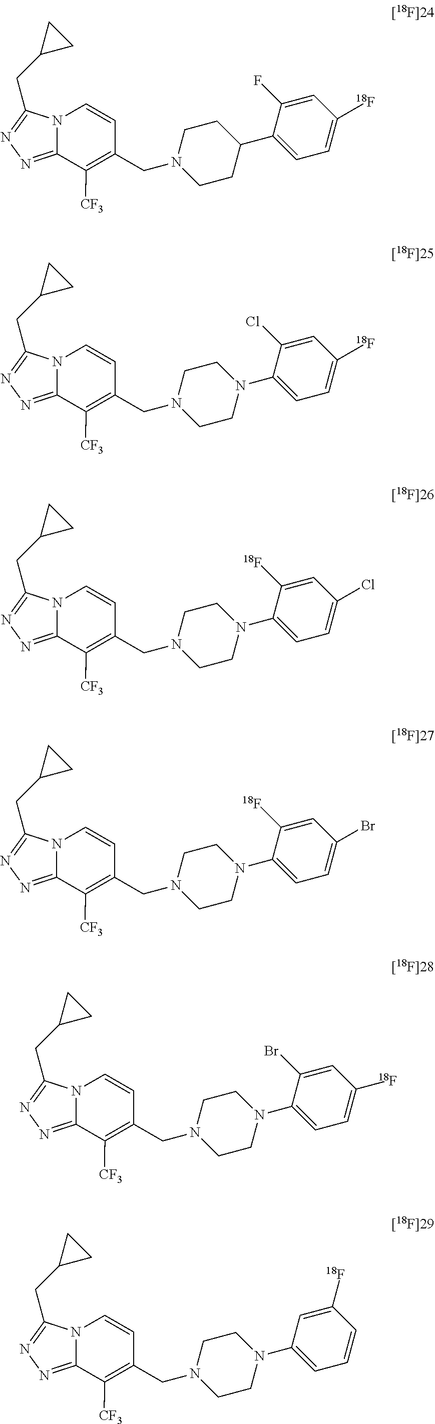 [SMILES: Fc1cc([18F])ccc1C1CCN(Cc2ccn3c(CC4CC4)nnc3c2C(F)(F)F)CC1 ; FC(F)(F)c1c(CN2CCN(c3ccc([18F])cc3Cl)CC2)ccn2c(CC3CC3)nnc12 ; FC(F)(F)c1c(CN2CCN(c3ccc(Cl)cc3[18F])CC2)ccn2c(CC3CC3)nnc12 ; FC(F)(F)c1c(CN2CCN(c3ccc(Br)cc3[18F])CC2)ccn2c(CC3CC3)nnc12 ; FC(F)(F)c1c(CN2CCN(c3ccc([18F])cc3Br)CC2)ccn2c(CC3CC3)nnc12 ; FC(F)(F)c1c(CN2CCN(c3cccc([18F])c3)CC2)ccn2c(CC3CC3)nnc12]

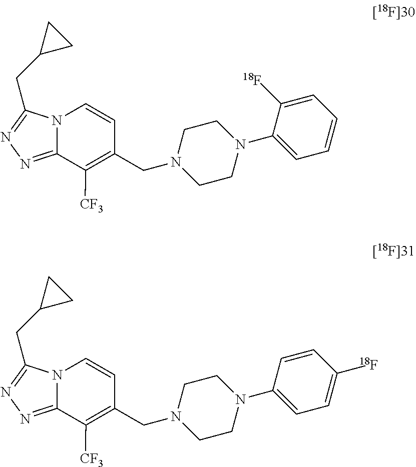 [SMILES: FC(F)(F)c1c(CN2CCN(c3ccccc3[18F])CC2)ccn2c(CC3CC3)nnc12 ; FC(F)(F)c1c(CN2CCN(c3ccc([18F])cc3)CC2)ccn2c(CC3CC3)nnc12]

or a pharmaceutically acceptable salt thereof.

In some embodiments, the compound of Formula (II) is:

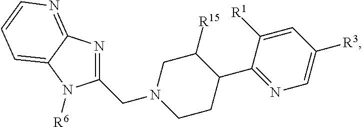

or a pharmaceutically acceptable salt thereof, wherein R¹, R³, R⁶, and R¹⁵ are as described herein.

In some embodiments, the compound of Formula (II) is:

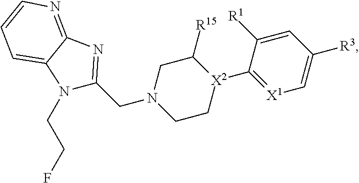

or a pharmaceutically acceptable salt thereof, wherein R¹, R³, X¹, X², and R¹⁵ are as described herein.

In some embodiments, the compound of Formula (II) is:

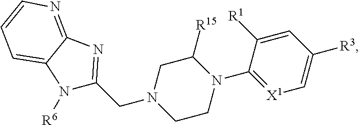

or a pharmaceutically acceptable salt thereof, wherein R¹, R³, R⁶, X¹, and R¹⁵ are as described herein.

In some embodiments, the compound of Formula (II) is:

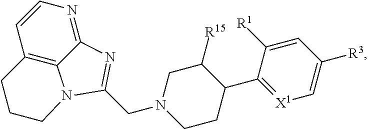

or a pharmaceutically acceptable salt thereof, wherein R¹, R³, X¹, and R¹⁵ are as described herein.

In some embodiments, the compound of Formula (II) is:

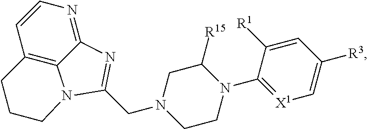

or a pharmaceutically acceptable salt thereof, wherein R¹, R³, X¹, and R¹⁵ are as described herein.

In some embodiments, the compound of Formula (II) is:

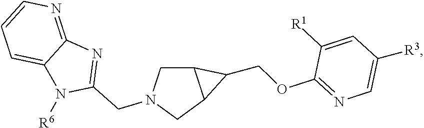

or a pharmaceutically acceptable salt thereof, wherein R¹, R³, and R⁶ are as described herein.

In some embodiments, the compound of Formula (II) is:

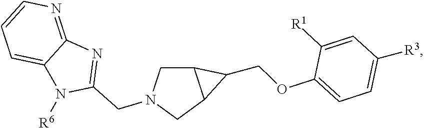

or a pharmaceutically acceptable salt thereof, wherein R¹, R³, and R⁶ are as described herein.

In some embodiments, the compound of Formula (II) is:

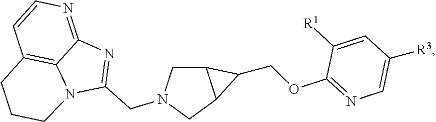

or a pharmaceutically acceptable salt thereof, wherein R¹ and R³ are as described herein.

In some embodiments, the compound of Formula (II) is:

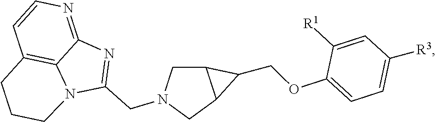

or a pharmaceutically acceptable salt thereof, wherein R¹ and R³ are as described herein.

In some embodiments, the compound of Formula (II) is:

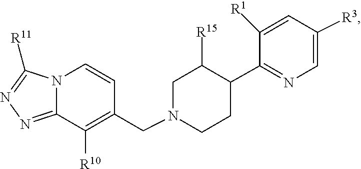

or a pharmaceutically acceptable salt thereof, wherein R¹, R³, R¹⁵, R¹⁰, and R¹¹ are as described herein.

In some embodiments, the compound of Formula (II) is:

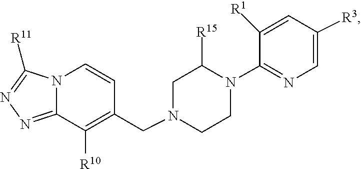

or a pharmaceutically acceptable salt thereof, wherein R¹, R³, R¹⁵, R¹⁰, and R¹¹ are as described herein.

In some embodiments, the compound of Formula (II) is selected from any one of the following compounds:

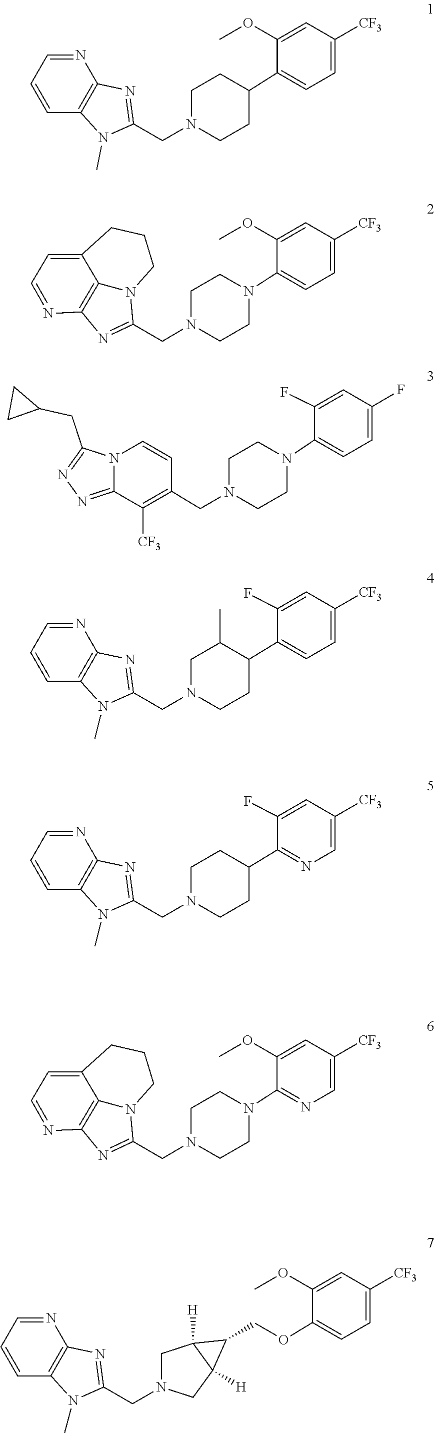

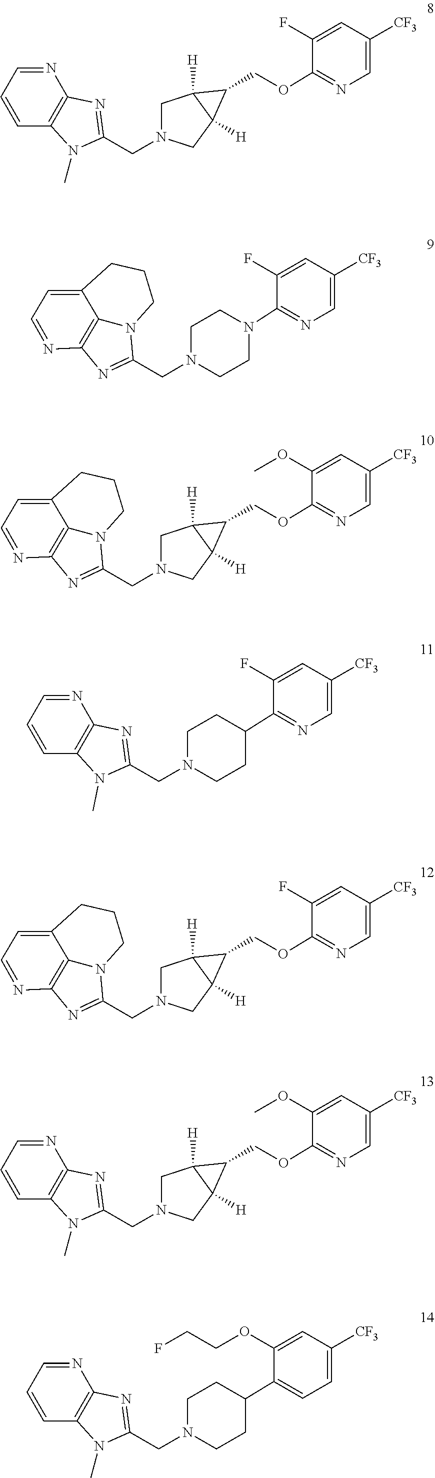

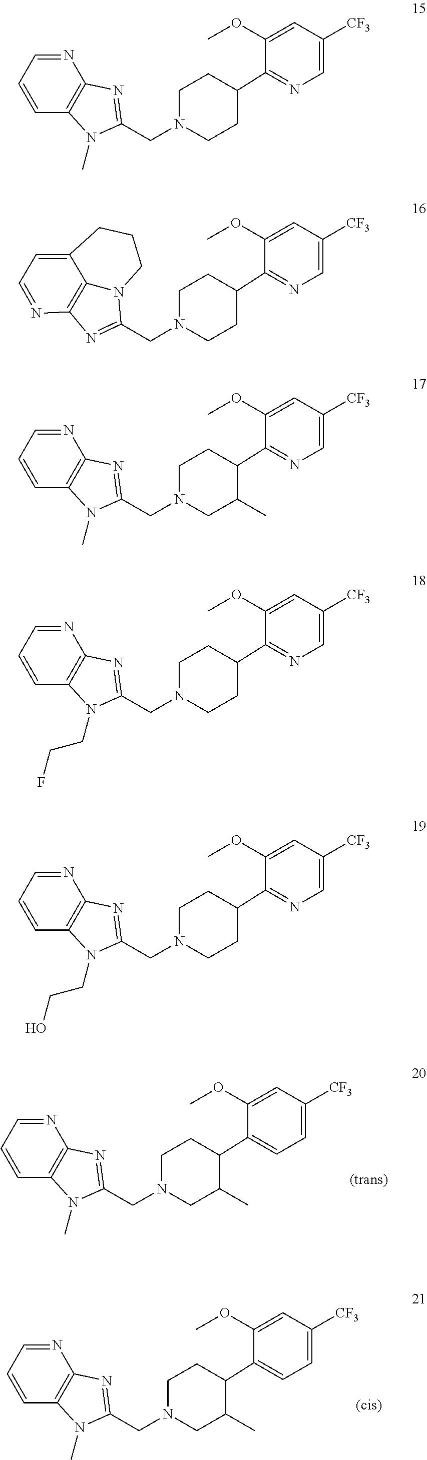

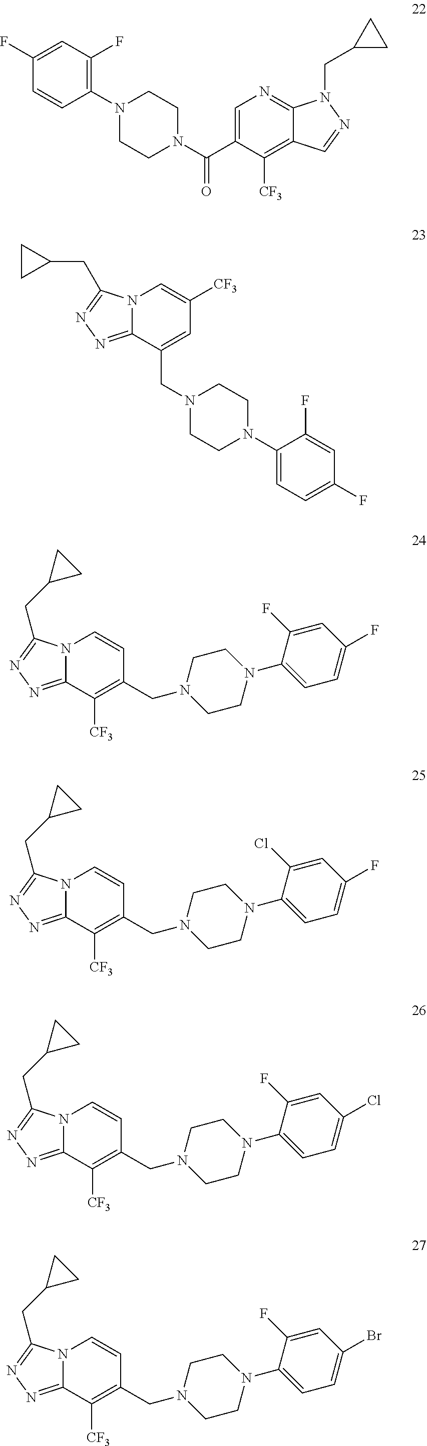 [SMILES: O=C(c1cnc2c(cnn2CC2CC2)c1C(F)(F)F)N1CCN(c2ccc(F)cc2F)CC1 ; Fc1ccc(N2CCN(Cc3cc(C(F)(F)F)cn4c(CC5CC5)nnc34)CC2)c(F)c1 ; Fc1ccc(N2CCN(Cc3ccn4c(CC5CC5)nnc4c3C(F)(F)F)CC2)c(F)c1 ; Fc1ccc(N2CCN(Cc3ccn4c(CC5CC5)nnc4c3C(F)(F)F)CC2)c(Cl)c1 ; Fc1cc(Cl)ccc1N1CCN(Cc2ccn3c(CC4CC4)nnc3c2C(F)(F)F)CC1 ; Fc1cc(Br)ccc1N1CCN(Cc2ccn3c(CC4CC4)nnc3c2C(F)(F)F)CC1]

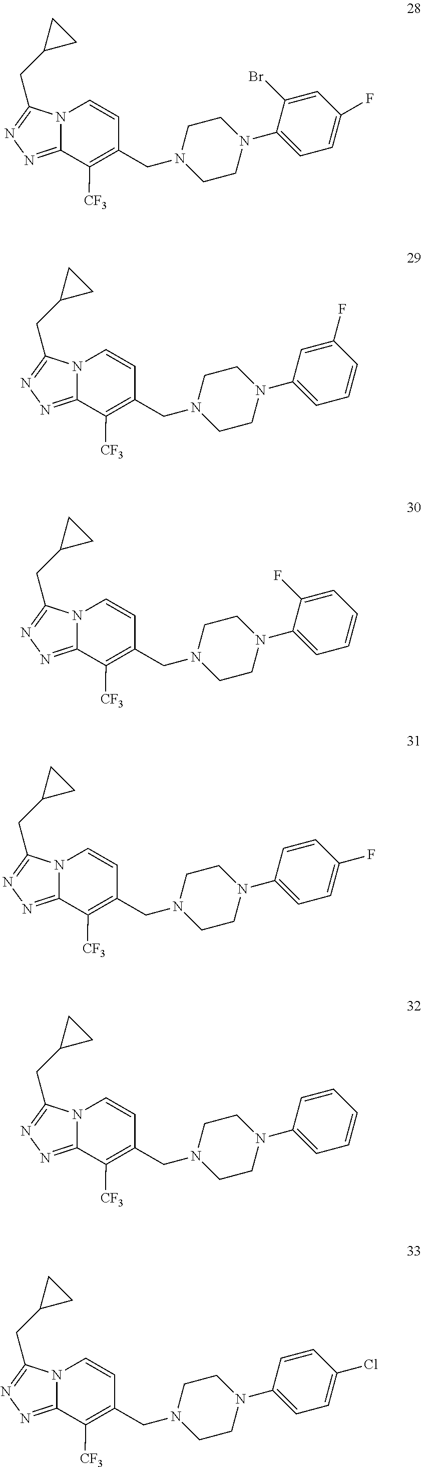 [SMILES: Fc1ccc(N2CCN(Cc3ccn4c(CC5CC5)nnc4c3C(F)(F)F)CC2)c(Br)c1 ; Fc1cccc(N2CCN(Cc3ccn4c(CC5CC5)nnc4c3C(F)(F)F)CC2)c1 ; Fc1ccccc1N1CCN(Cc2ccn3c(CC4CC4)nnc3c2C(F)(F)F)CC1 ; Fc1ccc(N2CCN(Cc3ccn4c(CC5CC5)nnc4c3C(F)(F)F)CC2)cc1 ; FC(F)(F)c1c(CN2CCN(c3ccccc3)CC2)ccn2c(CC3CC3)nnc12 ; FC(F)(F)c1c(CN2CCN(c3ccc(Cl)cc3)CC2)ccn2c(CC3CC3)nnc12]

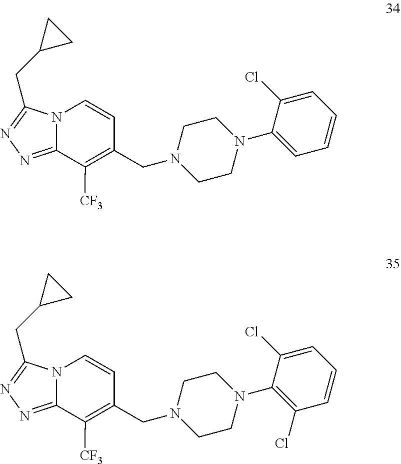 [SMILES: FC(F)(F)c1c(CN2CCN(c3ccccc3Cl)CC2)ccn2c(CC3CC3)nnc12 ; FC(F)(F)c1c(CN2CCN(c3c(Cl)cccc3Cl)CC2)ccn2c(CC3CC3)nnc12]

or a pharmaceutically acceptable salt thereof.

### Pharmaceutically Acceptable Salts

In some embodiments, a salt of any one of the compounds of the present disclosure is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, (3-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Methods of Use

In one general aspect, the present application relates to compounds of formula (I) useful in imaging techniques, diagnosing and monitoring treatment of various diseases and conditions described herein. Such compounds are labeled in so far as each compound includes at least one ¹⁸F radioisotope or at least one ¹¹C isotope.

### Methods of Diagnosis, Imaging, and Monitoring Treatment:

As the mGluR2 receptor has increasing attention within the pharmaceutical industry as a target for treatment of neurological and psychiatric disorders, developing a suitable PET tracer for imaging mGluR2 provides the capability of measuring biological process at molecular level in vivo. PET has become an important clinical diagnostic and research modality, and also a valuable technology in drug discovery and development. PET offers picomolar sensitivity and is a fully translational technique that requires specific probes radiolabeled with a usually short-lived positron-emitting radionuclide. Carbon-11 (radioactive half-life (t_{1/2})=20.4 min) and fluorine-18 (ti/2=109.7 min) are the most commonly used radionuclides in PET imaging. PET has provided the capability of measuring biological processes at the molecular and metabolic levels in vivo by the detection of the photons formed as a result of the annihilation of the emitted positrons.

As a noninvasive medical and molecular imaging technique and a powerful tool in neurological research, PET offers the possibility of visualizing and analyzing the target receptor expression under physiological and pathophysiological conditions. PET has often been used to detect disease-related biochemical changes before the disease-associated anatomical changes can be found using standard medical imaging modalities.

Moreover, PET tracers serve as invaluable biomarkers during the clinical development of potential therapeutics, in which the receptor occupancy of potential drug candidates in the brain is measured. In vivo receptor occupancy can help to answer many vital questions in the drug discovery and development process, such as whether potential drugs reach their molecular targets, the relationship between therapeutic dose and receptor occupancy, the correlation between receptor occupancy and plasma drug levels, and the duration of time the drug remains at its target.

Despite the great wealth of information that such probes can provide, the potential of PET strongly depends on the availability of suitable PET radiotracers. However, existing tracer discussed earlier suffer from serious drawbacks, including off-target binding, low BBB-penetration, and undesirable interaction with brain efflux pumps. The compounds within the present claims cross the BBB quickly and are mainly accumulated in the cortex, striatum, thalamus, hippocampus and cerebellum, which were reported as the mGluR2-rich regions of the rat brain, do not engage in off-target binding, and do not interact with brain efflux pumps.

In some embodiments, the present disclosure provides a method of identifying and quantifying mGluR2 density in the brain of a subject. This may be attained, for example, by imaging the brain. A method of imaging the brain comprises (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. Since ¹⁸F or ¹¹C within the compound of Formula (I) is a positron emitting radioisotope, the suitable imaging techniques include positron emission tomography (PET) and its modifications. As such, the imaging technique may be selected from positron emission tomography (PET) imaging, positron emission tomography with computer tomography (PET/CT) imaging, and positron emission tomography with magnetic resonance (PET/MRI) imaging, as well as other suitable methods.

In some embodiments, the present disclosure provides a method of diagnosing a psychiatric or a neurological disorder (e.g., psychiatric or neurological disorder in which mGluR2 is implicated) in a subject, the method comprising (i) administering to the subject an effective amount of a compound of Formula (I), or a pharmaceutically acceptable salt thereof, or a pharmaceutical composition comprising same; (ii) waiting a time sufficient to allow the compound to accumulate in the brain to be imaged (e.g., 1 min, 5 min, 10 min, 15 min, or 30 min), and (iii) imaging the brain with an imaging technique. The method may also comprise comparing images obtained from subjects exhibiting the symptoms of the disease or condition with the images obtained from healthy subjects. In one example, loss or overabundance of mGluR2 receptors in the brain of the subject may be indicative of a neurodegenerative disease (e.g., Alzheimer's disease or Parkinson's disease) or a psychiatric disease (e.g., schizophrenia or depression), or a related condition.

In some embodiments, the mGluR2-selective PET radiotracers of Formula (I) within the present claims are useful to study the role of mGluR2 in health and disease conditions. In some embodiments, the present disclosure provides a method of supporting the clinical development of potential therapeutics, in which the receptor occupancy of potential drug candidates such as mGluR2 allosteric modulators in the brain is measured. In vivo receptor occupancy can help to answer many vital questions in the drug discovery and development process such as whether potential drugs reach their molecular targets, the relationship between therapeutic dose and receptor occupancy, the correlation between receptor occupancy and plasma drug levels, and the duration of time the drug remains at its target.

In yet other embodiments, the present disclosure provides a method of monitoring treatment of a psychiatric or a neurological disorder (e.g., a psychiatric or a neurological disorder in which mGluR2 is implicated) in a subject, the method comprising (i) administering to the subject an effective amount of a compound of Formula (I), or a pharmaceutically acceptable salt thereof, or a pharmaceutical composition comprising same, (ii) waiting a time sufficient to allow the compound of Formula (I) to accumulate in a brain of the subject (e.g., 5 min, 15 min, or 30 min); (iii) imaging the brain of the subject with an imaging technique; (iv) administering to the subject a therapeutic agent in an effective amount to treat the psychiatric or the neurological disorder (e.g., levodopa or an experimental drug substance for treating AD, PD, schizophrenia, epilepsy, or depression); (v) after (iv), administering to the subject an effective amount of a compound of Formula (I), or a pharmaceutically acceptable salt thereof; (vi) waiting a time sufficient to allow the compound of Formula (I) to accumulate in the brain of the subject (e.g., 5 min, 15 min, or 30 min); (vii) imaging the brain of the subject with an imaging technique; and (viii) comparing the image of step (iii) and the image of step (vii). In one example, attaining overabundance of mGluR2 receptors in the brain of the subject, as determined by comparing the images, is indicative of successful treatment of the neurodegenerative disease. Suitable examples of diseases the treatment of which can be monitored according to the methods of the present disclosure include any of the diseases described herein. One particular example is schizophrenia. Other suitable examples include AD, PD, pain, psychosis, epilepsy, anxiety, depression, drug abuse, smoking cessation, dyskinesia, Lewy body disease, Prion disease, motor neuron disease (MND), and Huntington's disease.

### Methods of Modulating a Receptor

L-Glutamate is the most abundant excitatory neurotransmitter in the central nervous system (CNS) of vertebrates and probably mediates more than 50% of all synapses. Two major classes of receptors, ionotropic glutamate receptors (iGluRs) and metabotropic glutamate receptors (mGluRs), as well as transporters are involved in glutamate signaling. iGluRs, including the N-methyl-d-aspartate (NMDA), a-amino-3-hydroxy-5-methyl-4-isoxazole-propionate (AMPA), and kainite receptors are ligand-gated ion channels that mediate fast synaptic transmission. The mGluRs modulate the presynaptic glutamate release and/or postsynaptic effects of glutamate. mGluRs belong to class C of the G-protein-coupled receptor (GPCR) super family, which can be further divided into three subgroups including eight known receptor sub-types (group I: mGluR1 and mGluR5, group II: mGluR2 and mGluR3, and group III: mGluR4, mGluR6, mGluR7, and mGluR8) based on their structural similarity, ligand specificity, and preferred coupling mechanism. mGluRs are involved in glutamate signaling in almost every excitatory synapse in CNS, and they have distinctive biodistribution in CNS depending on subtypes and subgroups. Group II metabotropic glutamate receptors (mGluR2 and mGluR3) are widely expressed in the forebrain and localized presynaptically, where they negatively modulate glutamate and GABA release. Initial research and drug discovery efforts had focused on pharmacological ligands for mGluR2/3, which had largely been competitive orthosteric ligands including agonists and antagonists. These competitive orthosteric ligands possess extremely high potencies or binding affinities for the group, but poor selectivity within the group. A number of extremely potent group II mGluR selective agonists have been published in literature and developed for the treatment of anxiety and schizophrenia in preclinical and clinical studies. While normalization of glutamate levels through the use of mGluR2/3 agonists had shown comparable efficacy to conventional antipsychotic drugs for the treatment of schizophrenia, the preclinical studies revealed that the antipsychotic effect of mGluR2/3 agonists was absent in mGluR2 knockout mice but not mGluR3 knockout mice, suggesting the antipsychotic effects might be mediated via the mGluR2 but not mGluR3 receptor and even the effect of mGluR2 and mGluR3 might be different/opposite.

Because of the high degree of homology at the orthosteric sites of group II mGluRs, selective mGluR2 agonists have been difficult to design. Recently developed allosteric modulators have changed glutamate related drug development. Allosteric modulators are small molecules capable of enhancing agonist or antagonist mediated receptor activity while possessing no or less intrinsic agonist or antagonist activity. Relative to classical mGluR agonists and antagonists, the PAMs and NAMs offer improved selectivity versus other mGluRs and chemical tractability, and may reduce receptor desensitization specificity.

It is important to develop allosteric mGluR2-selective ligands for the diagnosis and treatment of neurological and psychiatric disorders. In recent years, several pharmaceutical companies and research groups have focused on developing PAMs and NAMs of mGluR2 as therapeutic drugs for different neurological conditions. Targeting mGluR2 with allosteric modulators has advantages over orthosteric ligands such as improved selectivity and better tolerability, which may offer enhanced therapeutic effects as well as improved side-effect profiles. It has been shown that enhancement or inhibition of mGluR functions has different biological response, which can be related to different diseases (such as those described herein). For example, it has been suggested that PAMs of mGluR2 can be related to therapeutic approaches of pain, schizophrenia, and drug abuse; while NAMs can be related to the therapeutics of cognitive disorders like AD.

In some embodiments, the present disclosure provides a method of modulating (e.g., positively allosterically modulating) mGluR2 in a cell, the method comprising contacting the cell with an effective amount of a compound of the present disclosure (e.g., Formula (I) or (II)), or a pharmaceutically acceptable salt thereof, or a pharmaceutical composition comprising same. In some embodiments, the contacting occurs in vitro, in vivo, or ex vivo. In some embodiments, the cell is a neuron. In some embodiments, the modulation is selective with respect to mGluR2, as opposed to other mGluR receptors (e.g., the modulation is 10×, 20×, 50×, 100×, or 1000× more selective with respect to mGluR2).

In some embodiments, the present disclosure provides a method of modulating (e.g., positively allosterically modulating) mGluR2 in a subject, the method comprising administering to the subject an effective amount of a compound of the present disclosure (e.g., Formula (I) or (II)), or a pharmaceutically acceptable salt thereof, or a pharmaceutical composition comprising same.

### Methods of Treating a Disease or Condition

In some embodiments, the compounds and compositions of the present disclosure are useful in treating psychiatric or neurodegenerative disease or disorder where mGluR2 is implicated in the pathology of the disease or condition. Suitable examples of neurodegenerative disease or disorder include Parkinson's disease (including associated deficits in motor system such as akinesia, bradykinesia, and dyskinesia), Alzheimer's disease, dyskinesia, Lewy body disease, Prion disease, motor neuron disease (MND), Huntington's disease, amyotrophic lateral disorder, ischemia, ischemic brain damage, and a traumatic brain injury. Suitable examples of psychiatric disease or disorder include anxiety disorders, depression, drug addiction, pain, schizophrenia, psychosis, anxiety, drug abuse, smoking cessation, and epilepsy.

Other examples of diseases and conditions treatable by the compounds of the present disclosure include cerebral deficits subsequent to cardiac bypass surgery and grafting, stroke, cerebral ischemia, spinal cord trauma, head trauma, perinatal hypoxia, cardiac arrest, hypoglycemic neuronal damage, dementia, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, convulsions, migraine, urinary incontinence, substance tolerance, substance withdrawal, mood disorders, trigeminal neuralgia, hearing loss, tinnitus, macular degeneration of the eye, emesis, brain edema, tardive dyskinesia, sleep disorders, attention deficit/hyperactivity disorder, and conduct disorder.

In some embodiments, the compounds and compositions of the present disclosure are useful in treating brain cancer. Suitable examples of brain cancer include glioblastoma and medulloblastoma. In some embodiments, the present disclosure provides a use of a compound or a composition as described herein in the manufacture of a medicament for the treatment of any one of the disease or conditions described herein.

### Combinations

The compounds of the present disclosure can be used on combination with at least one medication or therapy useful, e.g., in treating or alleviating symptoms of a psychiatric or a neurological condition. Suitable examples of such medications include levodopa (L-dopa), carbidopa, safinamide, dopamine agonists (e.g., ropinirole, pramipexole, rotigotine), amantadine, trihexyphenidyl, benztropine, selegiline, rasagiline, tolcapone, and entacapone, or a pharmaceutically acceptable salt thereof. Other examples include antidepressants (e.g., SSRIs, SNRIs, or tricyclic antidepressants) and antipsychotics (e.g., aripiprazole, fluphenazine, haloperidol, paliperidone, or risperidone).

The compound of the present disclosure may be administered to the patient simultaneously with the additional therapeutic agent (in the same dosage form or in different dosage forms) or consecutively (the additional therapeutic agent may be administered before or after administration of the compound of the present disclosure).

### Pharmaceutical Compositions

The present application also provides pharmaceutical compositions comprising an effective amount of a compound of the present disclosure (e.g., Formula (I) or Formula (II)) disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethyleneglycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of the present disclosure (e.g., a compound of Formula (I) or Formula (II)) is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of the compound (e.g., Formula (I) or Formula (II)) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound of Formula (I) or Formula (II) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present disclosure also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Definitions

As used herein, the term “about” means “approximately” (e.g., plus or minus approximately 10% of the indicated value).

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the disclosure, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula —N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “thio” refers to a group of formula —SH.

As used herein, the term “Cₙ₋ₘ alkylthio” refers to a group of formula —S-alkyl, wherein the alkyl group has n to m carbon atoms. In some embodiments, the alkyl group has 1 to 6, 1 to 4, or 1 to 3 carbon atoms.

As used herein, “halo” refers to F, Cl, Br, or I. In some embodiments, a halo is F, Cl, or Br.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Ring-forming carbon atoms of a cycloalkyl group can be optionally substituted by 1 or 2 independently selected oxo or sulfide groups (e.g., C(O) or C(S)). Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, or 10 ring-forming carbons (C₃₋₁₀). In some embodiments, the cycloalkyl is a C₃₋₁₀ monocyclic or bicyclic cyclocalkyl. In some embodiments, the cycloalkyl is a C₃₋₇ monocyclic cyclocalkyl. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. In some embodiments, cycloalkyl is cyclopropyl, cyclobutyl, cyclopentyl, or cyclohexyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 1-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyO isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the mGluR2 with a compound of the disclosure includes the administration of a compound of the present disclosure to an individual or patient, such as a human, having mGluR2, as well as, for example, introducing a compound of the disclosure into a sample containing a cellular or purified preparation containing the mGluR2.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “radioisotope” refers to an atom having an atomic mass or mass number different from the atomic mass or mass number typically found in nature (i.e., naturally occurring).

As used herein, the term “isotopic enrichment factor” refers to the ratio between the isotopic abundance and the natural abundance of a specified isotope.

“D” and “d” both refer to deuterium. A compound of the present disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“¹⁸F” refers to the radioisotope of fluorine having 9 protons and 9 neutrons. “F” refers to the stable isotope of fluorine having 9 protons and 10 neutrons (i.e., the “¹⁹F isotope”). A compound of the present disclosure has an isotopic enrichment factor for each designated ¹⁸F atom of at least 3500 (52.5% ¹⁸F incorporation at each designated ¹⁸F atom), at least 4000 (60% ¹⁸F incorporation), at least 4500 (67.5% ¹⁸F incorporation), at least 5000 (75% ¹⁸F), at least 5500 (82.5% ¹⁸F incorporation), at least 6000 (90% ¹⁸F incorporation), at least 6333.3 (95% ¹⁸F incorporation), at least 6466.7 (97% ¹⁸F incorporation), at least 6600 (99% ¹⁸F incorporation), or at least 6633.3 (99.5% ¹⁸F incorporation).

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “radioisotope” refers to an atom having an atomic mass or mass number different from the atomic mass or mass number typically found in nature (i.e., naturally occurring).

As used herein, the term “isotopic enrichment factor” refers to the ratio between the isotopic abundance and the natural abundance of a specified isotope.

“D” and “d” both refer to deuterium. A compound of the present disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

“¹⁸F” refers to the radioisotope of fluorine having 9 protons and 9 neutrons. “F” refers to the stable isotope of fluorine having 9 protons and 10 neutrons (i.e., the “¹⁹F isotope”). A compound of the present disclosure has an isotopic enrichment factor for each designated ¹⁸F atom of at least 3500 (52.5% ¹⁸F incorporation at each designated ¹⁸F atom), at least 4000 (60% ¹⁸F incorporation), at least 4500 (67.5% ¹⁸F incorporation), at least 5000 (75% ¹⁸F), at least 5500 (82.5% ¹⁸F incorporation), at least 6000 (90% ¹⁸F incorporation), at least 6333.3 (95% ¹⁸F incorporation), at least 6466.7 (97% ¹⁸F incorporation), at least 6600 (99% ¹⁸F incorporation), or at least 6633.3 (99.5% ¹⁸F incorporation).

“¹¹C” refers to the radioisotope of carbon having 6 protons and 5 neutrons. “C” refers to the stable isotope of carbon having 6 protons and 6 neutrons (i.e., the “¹²C isotope”). A compound of the present disclosure has an isotopic enrichment factor for each designated ¹¹C atom of at least 3500 (52.5% ¹¹C incorporation at each designated ¹¹C atom), at least 4000 (60% ¹¹C incorporation), at least 4500 (67.5% ¹¹C incorporation), at least 5000 (75% ¹¹C), at least 5500 (82.5% ¹¹C incorporation), at least 6000 (90% ¹¹C incorporation), at least 6333.3 (95% ¹¹C incorporation), at least 6466.7 (97% ¹¹C incorporation), at least 6600 (99% ¹¹C incorporation), or at least 6633.3 (99.5% ¹¹C incorporation).

### Examples

### Abbreviation Used

BBB, blood-brain barrier; VFTD, Venus flytrap domain; 7-TM, seven transmembrane; CRD cysteine rich domain; POOL, Partial Order Optimum Likelihood; EL2, extracellular loop 2; PDB, Protein Data Bank; PAM, positive allosteric modulator; NAM, negative allosteric modulator; MW, molecular weight; tPSA, topological polar surface area; Clᵢₙₜ, the intrinsic clearance; G₀ adenylate cyclase inhibitory G-protein; MWCO, molecular weight cut off; ND, not determined; PBL, Polar brain lipid; PBL, porcine polar brain lipid; P-gp, P-glycoprotein; PAMPA, parallel artificial membrane permeability assay; Pₑ, effective permeability; EOS, end of synthesis. SWFI, sterile water for injection; TAC, time-activity curve; SUV, standardized uptake value; USP, United States Pharmacopeia; % ID/g, percentage of injected dose per gram of wet tissue.

### General Methods

All reagents and starting materials were obtained from the commercial sources including Sigma-Aldrich (St. Louis, Mo.), Thermo Fisher Scientific, Oakwood Products, Inc., Matrix Scientific, Acros Organics and used as received. The reactions were monitored by TLC using a UV lamp monitored at 254 nm. If necessary, the reactions were also checked by LC-MS using the Agilent 1200 series HPLC system coupled with a multi-wavelength UV detector and a model 6310 ion trap mass spectrometer (Santa Clara, Calif.) equipped with an Agilent Eclipse C8 analytical column (150 mm×4.6 mm, 5 μm). Elution was with a 0.1% formic acid solution of water (A) and acetonitrile (B). The silica gel used in flash column chromatography was from Aldrich (Cat. 60737, pore size 60 Å, 230-400 mesh). Flash chromatography was also performed with a CombiFlash Rf Purification System (Teledyne Isco) using a Silica ReadySep Rf column. The products were identified by LC-MS as well as ¹FI NMR, ¹³C NMR and ¹⁹F NMR using a Varian 500 MHz spectrometer. All NMR samples were dissolved in chloroform-d (CDCl3), methanol-d4 (CD₃OD) or DMSO-d6 [(CD₃)₂SO] containing tetramethylsilane as a reference standard. Chemical shifts were expressed as ppm and calculated downfield or upfield from the NMR signal of reference standard. J was expressed as Hz, and its splitting patterns were reported as s, d, t, q, or m. FIRMS was obtained from the Fligh-Resolution Mass Spectrometry Facility at the University California, Riverside, using electrospray ionization (ESI)/atmospheric pressure chemical ionization (APCI) technique (Agilent Time of Flight (TOF) LC-MS). Unless otherwise specified, the purities of all new compounds were over 95% determined by HPLC.

### Molecular Modeling

Model Structure: The mGluR2 receptor model structure was built in YASARA [See ref. 40] using a series of structures from the Protein Data Bank (PDB). These structures were obtained after a BLAST [See ref. 66] search of the mGluR2 sequence against the PDB. The model was built by manually selecting from these template structures with sequence homology to mGluR2. These templates are mGluR1 complexed with glutamate (PDB ID:1EWK) [See ref. 67], mGluR5 complexed with glutamate (PDB ID: 3LMK) [See ref. 68] and Metabotropic Glutamate Receptor 5 Apo Form (PDB ID 6N52) [see ref. 69]. Using these three structures as templates, a hybrid model for mGluR2 was built in YASARA.

Ligand Docking: To prepare the ligands for docking, the ligands were drawn on ChemDraw Professional 16.0 by PerkinElmer and were converted into PDB format in Avogadro 1.2 [See ref. 70]. These ligands were further optimized in Avogadro before docking. Docking was performed into the model structure with AutoDock [see ref. 48] embedded in YASARA [see ref. 40].

### Ca²⁺ Mobilization Assay

The Gq coupled receptors (mGluR1 and mGluR5) were tested using Ca²⁺ mobilization assay. mGluR1 stable cell lines were plated into poly-L-lysine (PLL) coated 384-well black clear bottom cell culture plates with complete Basal Medium Eagle (BME) buffer, which was composed of 10% dialyzed FBS, penicillin G (100 units/mL), streptomycin (100 μg/mL) with tetracycline (1 μg/mL) at density of 20,000 cells in 40 μl per well for overnight. On the other hand, HEK-293 Cells transiently transfected using the calcium phosphate method with cDNA encoding mGluR5 for 40 h were plated into the plate with complete BME at density of 20,000 cells in 40 μL per well for 8 h. mGluR1 stable cells or cells transiently expressing mGluR5 were incubated with 20 μL of the calcium dye (FLIPR Calcium 4 Assay Kit; Molecular Devices) diluted in the assay buffer (1×FIBSS, 2.5 mM probenecid, and 20 mM HEPES, pH 7.4) for 45 min at 37° C. and 15 min at room temperature. To measure agonist activity of receptors, the drug plates were prepared with different concentrations of test or reference compound at 3 times the desired final concentration. When measuring antagonist activity, another drug plate which contained EC₈₀ concentration of the reference drug was prepared. Once loaded in FLIPR (Molecular Devices), basal fluorescence was measured for 10 s, then 10 μL of test or reference compounds were added, followed by continued fluorescence measurement for an additional 120 s. Raw data were plotted as a function of molar concentration of the compound with Prism 5.0 (GraphPad Software).

cAMP assay

The Gi/o coupled receptors (mGluR2, mGluR3, mGluR4, mGluR6 and mGluR8) were tested using cAMP assay. Promega's split luciferase based GloSensor cAMP biosensor technology was used in determining Gi-GPCR mediated cAMP production in live cells. On the cells stably expressing mGluR2, mGluR3, mGluR4, mGluR6 or mGluR8, GloSensor cAMP DNA construct was transfected overnight. Cells were seeded into PLL coated 384-well white clear bottom cell culture plates with complete BME Buffer with Tetracycline (1 μg/mL) at a density of 20,000 cells for another 24 h. The cell medium was removed and then 20 μL of buffer was loaded. To measure the agonist activity, 10 μL of 3× test compound solution was added 15 min before addition of 10 μL of luciferin/isoproterenol mixture at a final concentration of 4 mM and 200 nM, respectively, followed by counting of the plate. To measure the PAM or antagonist activity, cells were pre-incubated with test compound for 15 min before addition of EC₂₀ or EC₈₀ concentration of a reference agonist for another 15 min. Then 10 μL of luciferin/isoproterenol mixture at a final concentration of 4 mM and 200 nM, respectively, was added for 15 min followed by counting of the plate. In these experiments, isoproterenol was used to activate endogenous (32 adrenergic receptors expressed in HEK293 T cells to activate the endogenous Gs protein. Luminescence was counted in a TriLux luminescence counter. Data were analyzed with Prism 5.0 (GraphPad software).

### Secondary Assays—Dose-Response Assays

Compounds were tested for their potency in dose-response experiments. Eight-point dose response curves were performed in duplicate twice on two separate lots of cells (sometimes a third curve might be needed if in the first experiment the range of concentrations used was outside of the active range). For antagonists, these curves were performed in the presence of the ECso concentration of the agonist. For each compound, the results from four replicates were averaged and then either EC₅₀ or IC₅₀ values were calculated by non-linear regression using the 4-parameter logistic equation. Results were reported as EC₅₀ or IC₅₀ values for each tested compound (and receptor) and include the EC₅₀ or IC₅₀ values of a known agonist or antagonist for comparison purposes.

### In Vitro Characterization

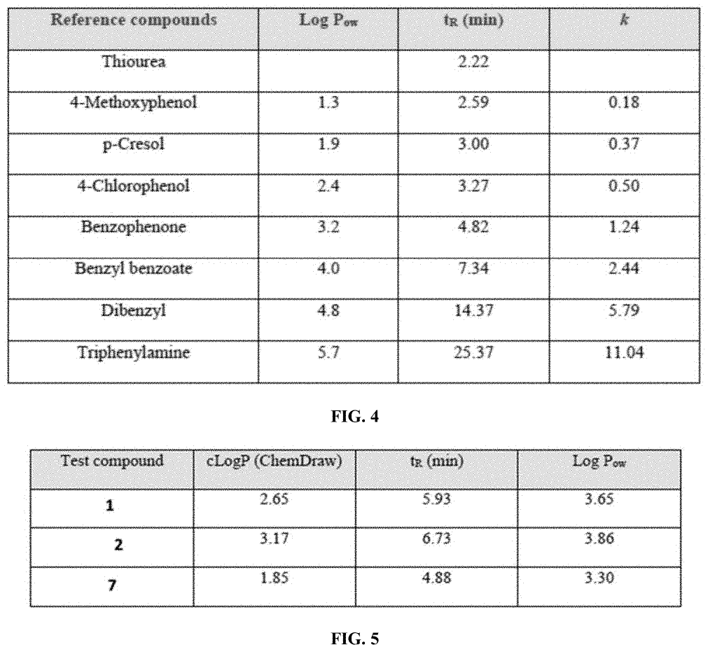

Determination of Log P The Log P was determined using a reversed-phase HPLC method. First, seven reference compounds were examined to obtain the linear regression of the log P against the log of capacity factors by the expression: log P_{ow}=a+b*log k. The Log P of these reference compounds was already been determined. The capacity factor k was calculated by the expression: k=(t_{R}−t₀)/t₀. The retention time t_{R} of test compound was determined on the HPLC (Agilent 1260 infinity II LC System, XTerra™ MS Cis 5μ 2.1×250 mm, methanol/water=75/25, 0.25 mL/min). The dead-time to was measured by using thiourea. All measurements were done with triplicate three parallels and results are given in FIG. 4. The linear regression equation of the Log P against the log of capacity factors was generated in Excel: log P_{ow}=3.049+2.429*log k, where R² was 0.9964. The retention time of compound 1, 2, and 7 was also determined on the HPLC under the same condition and each test was repeated three times (FIG. 5).

### Plasma Protein Binding Assay

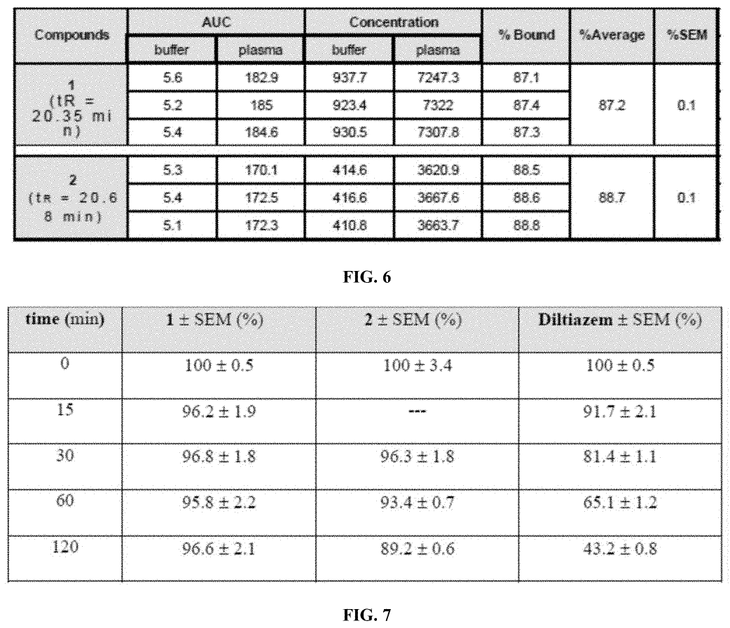

Disposable RED device inserts (product 90006) were from Thermos Scientific (Waltham, Mass.). Each insert was made of two side-by-side chambers separated by a vertical cylinder of dialysis membrane (MWCO 8,000) validated for minimal non-specific binding. A stock solution of the test compound in DMSO was spiked into the rat plasma to reach a concentration of 10 μM. 400 μL of sample solution was placed into the sample chamber of the RED device, and 600 μL of phosphate-buffered saline (PBS) was added to the buffer chamber of the RED device. Samples were prepared in triplicates. The plate was covered with aluminum sealing cover and incubated at 37° C. on an orbital shaker at approximately 200 rpm for 5 h. After incubation, 300 μL of post-dialysis samples from the buffer and sample chambers were transferred to different microcentrifuge tubes. To the buffer sample was added 300 μL of plasma, and an equal volume of buffer was added to the collected plasma sample. 600 μL of cold acetonitrile was added to the samples, and the samples were vortexed and incubated for 30 min on ice and then were centrifuged at 14000 rpm for 10 min. Supernatant was transferred to vial for HPLC analysis (XTerra™ MS C₁₈ 5μ, 2.1×250 mm column; Gradient elution from 5% to 90% B in 30 min; 0.1 M ammonium formate in water (A) and acetonitrile (B); UV 254 nm; 100 μL of injection volume). The percentage of the test bound compound was calculated as % Free=(Concentration in buffer chamber/Concentration in plasma chamber)×100%; % Bound=100%−% Free (FIG. 6).

### Plasma Stability

Compound stability in rat serum was examined using a published method. [See ref. 57] Rat serum (100 μL, Abeam, Inc, No. ab7488) and test compound or control compound (2.5 μL, 1 mM in DMSO) was added to the individual tube. The tube was vortexed and incubated at 37° C. During the incubation, aliquots of 50 μL samples were quenched with ice-cold acetonitrile at 0, 15, 30, 60, and 120 min time points, respectively. After mixing, the quenched samples were centrifuged, and the supernatant was withdrawn for analysis by HPLC (Agilent 1260 infinity II LC System, XTerra™ MS C₁₈, 5μ, 2.1×250 mm, 20 mM ammonium formate (A)/acetonitrile (B), 0.25 mL/min, gradient of 5% to 100% B). The samples were assayed at least three times. Compound 1-OH was used as internal standard while diltiazem was used as a positive control. The percentage remaining was calculated by (peak area at the specific time point)/(peak area at 0 min)×100% (FIG. 7).

### Microsomal Stability

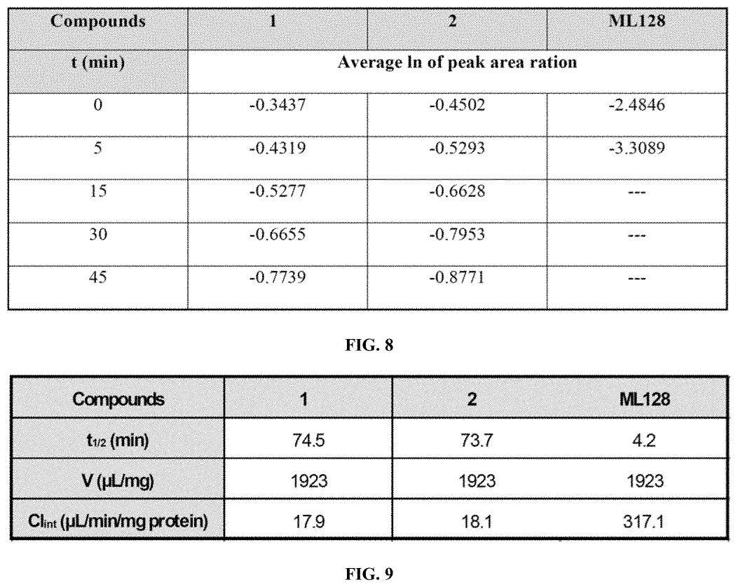

Compound stability in rat liver microsomal was tested using a published method. [see refs. 57, 58] In a vial 1.5 μL of test compound (1 mM in DMSO stock solution) was mixed with 432 μL of PBS (50 mM, pH 7.4). The mixture was kept at 37° C. for 10 min before adding 13 μL of Sprague-Dawley rat liver microsomes (Sigma-Aldrich, No. M9066). The vial was vortexed and shaken at 37° C. for 5 min, followed by addition of 50 μL of NADPH (10 mM in PBS stock solution) to start the reaction. The mixture was incubated at 37° C. for 0, 5, 15, 30, 45 min, respectively, and quenched by addition of 250 μL of ice-cold acetonitrile and 3 μL of the internal standard (0.5 mM in DMSO). The quenched solutions were centrifuged at 10,000 g for 15 min. The supernatant was collected and quantitated by RP-HPLC (Phenomenex Luna® column 5 μC₁₈, 100 Å, 250×4.6 mm; 0.7 mL/min, 15 min, Acetonitrile/water/0.1% FA). The procedure was repeated three times for each compound. Compound 1-OH was used as internal standard and compound ML128 served as positive control. The percentage of remaining intact test-compound was calculated by (peak area at the specific time point)/(peak area at 0 min)×100%. Each procedure was repeated three times (FIGS. 8 and 9). For FIG. 8, the natural logarithm (In) of peak area ratio (test compound peak area/internal standard peak area) was plotted against time and the gradient of the line was determined. Subsequently, the half-life (t_{1/2}) min was calculated as 0.693/k, where the elimination rate constant (k) equals to negative gradient. V (μL/mg) was obtained as a ratio of volume of incubation (μL)/protein in the incubation (mg). Finally, the intrinsic clearance (C_{Lint}) (μL/min/mg protein) was calculated as V×0.693/t_{1/2}.

### The Solution Stability

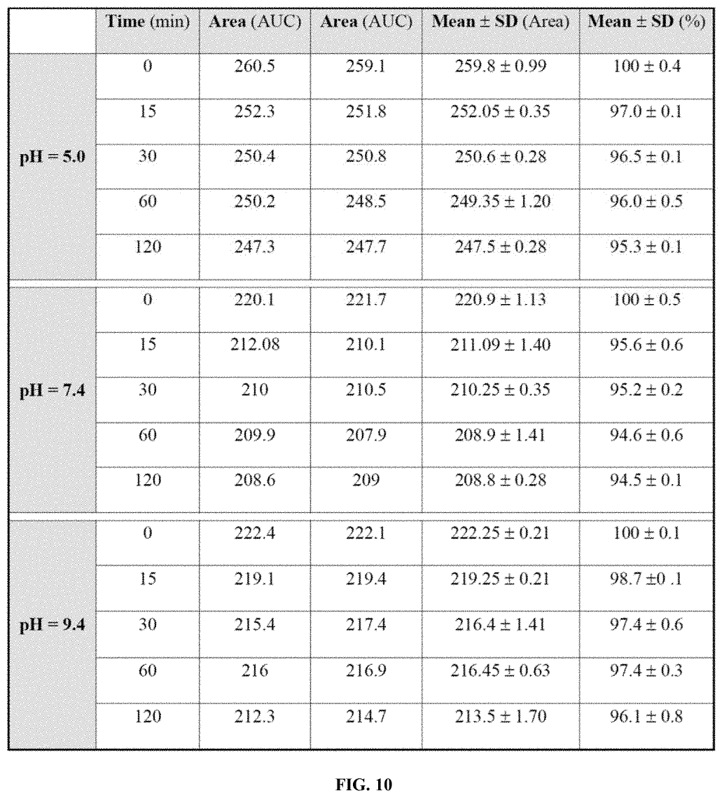

The solution stability of 3 was examined in the aqueous buffers at different pH values. 50 μL of compound in DMSO (0.25 mM) was added to the sodium acetate-KCl—HCl buffer (950 μL, 20 mM, pH 5.0), phosphate buffer (950 μL, 20 mM, pH 7.4), and boric acid-KCl—NaOH buffer (950 μL, 20 mM, pH 9.4), respectively. The mixtures were incubated for 2 h at 37° C. and analyzed by HPLC (Phenomenex Luna® column, 5 μm C₁₈, 100 Å, 250×4.6 mm, eluents: CH₃CN/H₂O in 0.1% formic acid). The area under curve (AUC) values of 13 was monitored at 0, 15, 30, 60, and 120 min time points (n=2, see FIG. 10).

### PAMPA-BBB (Parallel Artificial Membrane Permeation Assay)

Polar brain lipid (PBL) was purchased from Avanti Polar Lipids (Alabaster, Ala.). Theophylline, caffeine, and dodecane were purchased from Sigma-Aldrich. The 96-well acceptor filter plate (polyvinylidene difluoride membrane, pore size 0.45 μm) and the donor microplate were obtained from Merck Millipore Bioscience (Bedford, Mass.). Test compound was dissolved in DMSO at 5 mg/mL, and further diluted in phosphate buffer (pH 7.4) to obtain the sample solution at a final concentration of 25 μg/mL. The acceptor wells were coated with 4 μL of porcine polar brain lipid (PBL) in dodecane (20 mg/mL) before 200 μL of phosphate buffer was added. To the corresponding donor well, 300 μL of the sample solution (n=5) was added. The acceptor well was carefully put on the donor plate and kept for 18 h. After incubation, the acceptor plate was separated from the donor plate and the concentration of the test compounds in both acceptor and donor wells was determined using a UV plate reader (SpectraMax M Series Multi-Mode Microplate Readers). Verapamil (Pe=16×10⁻⁶ cm/s) and theophylline (Pₑ=0.12×10⁻⁶ cm/s) were used as positive and negative control compounds, respectively.

### P-Gp ATpase Assay (Pgp-Glo™ Assay)

P-gp ATPase activity was measured with the Pgp-Glo™ assay system with human P-gp membrane by following the manufacturer's instructions (Promega, Co. USA). The assay relies on the ATP dependence of the light-generating reaction of firefly luciferase. Briefly, 25 μg of P-gp membrane was incubated at 37° C. with one of these samples including Na₃VO₄ (100 μM), solvent control (0.1% DMSO), quercetin (100 μM), the test compound (200 μM), verapamil (100 μM), verapamil (100 μM) plus the test compound (100 μM). The ATPase reaction was initiated by addition of MgATP (5 mM) and followed by incubation for 40 min at 37° C. The reaction was stopped, and the remaining unmetabolized ATP was detected as a luciferase-generated luminescence signal by addition of ATP detection reagent. Following a room-temperature signal-stabilization period (20 min), luminescence was read on a Veritas microplate luminometer (Tuner Designs, San Francisco, Calif.). P-gp ATPase activity was presented as a drop-in luminescence of samples compared to that treated with Na₃VO₄.

### Whole Body Biodistribution Studies

The quantitative biodistribution of [¹¹C]1 was done using 16 healthy Sprague Dawley rats (weight 330-370 g). After anesthetization (2% isoflurane with oxygen flow of 1.5 L/min) the rats were administrated with the [¹¹C]1 (30-42 MBq (0.81-1.14 mCi) using tail vein injection and sacrificed by decapitation at the time points 5, 20, 30 or 40 min after administration of the radioactivity. The tissue samples including blood, midbrain, cerebellum, cortex, lung, heart, liver, spleen, kidney and muscle were rapidly collected into pre-weighted gamma-counting tubes and measured with standards (samples of [¹¹C]1) using PerkinElmer Wizard2 2480 gamma-counter. Tubes were weighted, and the net mass of the tissue samples was determined and the percent of the injected radioactivity (% ID/g) in the samples was calculated.

### In Vivo Characterization

Altogether twelve normal Sprague Dawley rats (male, 275-500 g) were used in sixteen studies to investigate in vivo imaging characteristics of [¹¹C]1. Four rats had control studies followed by the “blocking” studies while three rats had only “blocking” studies and 5 rats had only control studies to investigate specificity and sensitivity of [¹¹C]1. For the imaging studies rats were anesthetized with isoflurane/nitrous oxide (1.0-1.5% isoflurane, with oxygen flow of 1-1.5 L/min) and the tail vein was catheterized for administration of the imaging ligand ([¹¹C]1). The rats were adjusted into the scanner for imaging position (Triumph II Preclinical Imaging System, Trifoil Imaging, LLC, Northridge, Calif.). The vital signs such as heart rate and/or breathing were monitored throughout the imaging. Data acquisition of 60 min was started from the injection of radioligand [¹¹C]1 (20-41 MBq (0.54-1.11 mCi) i.v.). The “cold” compounds 1 and 7 were used to investigate specificity and sensitivity of [¹¹C]1 for the mGluR2. For injection 1 was dissolved into a saline solution with 10% DMSO, 5% Tween-20 and 85% PBS with a pH of 7.4 and 7 was dissolved into saline with 20% HP-B-CD with pH under 5.5. The “cold” compounds were administered (i.v., 4 mg/kg) 10 min before the radioactivity. CT scan was performed after every PET imaging study to obtain anatomical information and correction for attenuation. The PET imaging data were corrected for uniformity, scatter, and attenuation and processed by using maximum-likelihood expectation-maximization (MLEM) algorithm with 30 iterations to dynamic volumetric images (18×10″, 14×30″, 20×60″, 10×180″). CT data were reconstructed by the modified Feldkamp algorithm using matrix volumes of 512×512×512 and pixel size of 170 μm. The ROIs, i.e., whole brain, thalamus, cerebellum, striatum, and cortex, were drawn onto coronal PET slices according to the brain outlines as derived from the rat brain atlas and corresponding TACs (time-activity curves) were created by PMOD 3.2 (PMOD Technologies Ltd., Zurich, Switzerland). Percent changes between the control and blocking studies were calculated in the selected brain areas at the 10-30 min time window after injection of [¹¹C]1.

Preparation of mGluR2 Homology Model

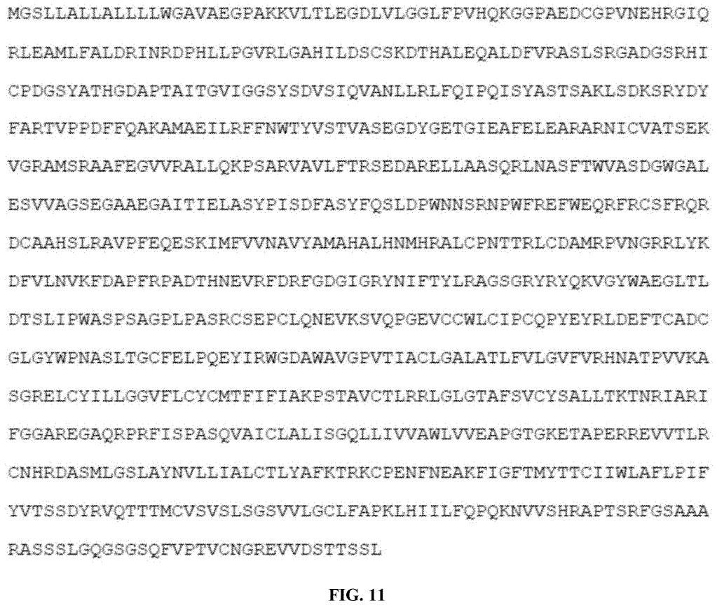

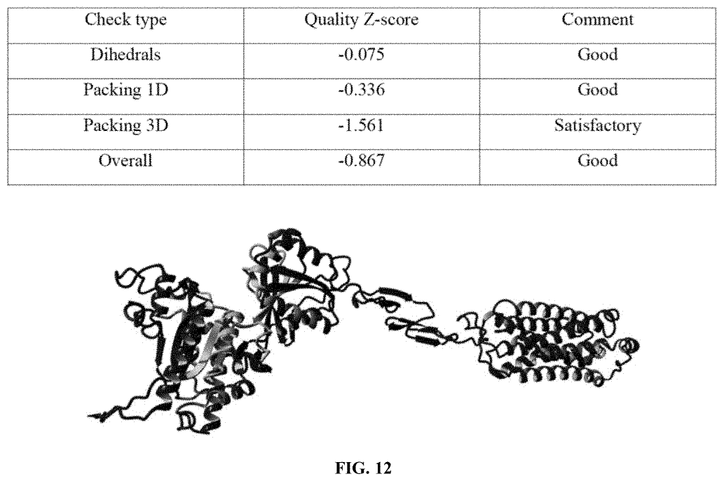

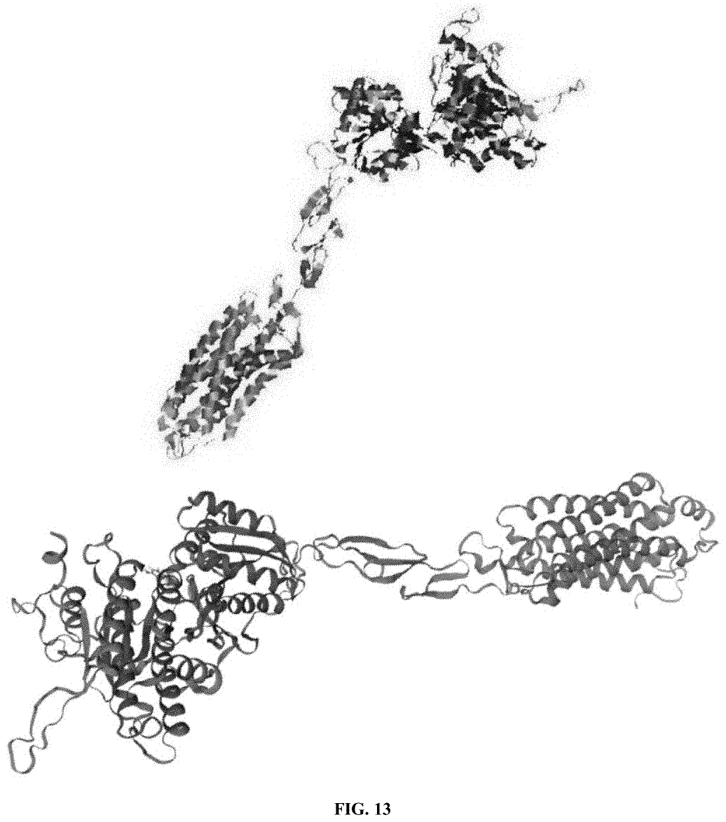

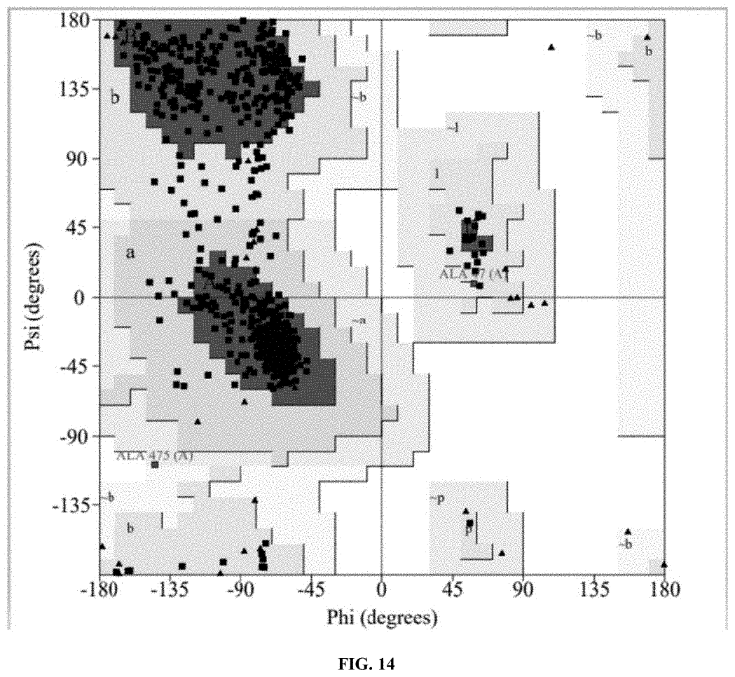

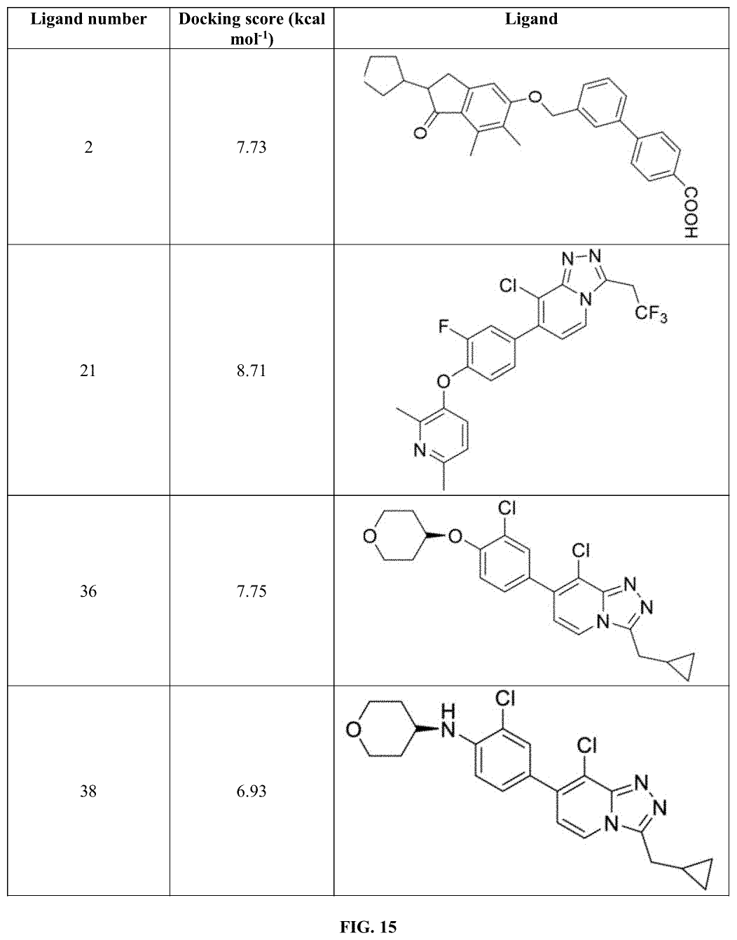

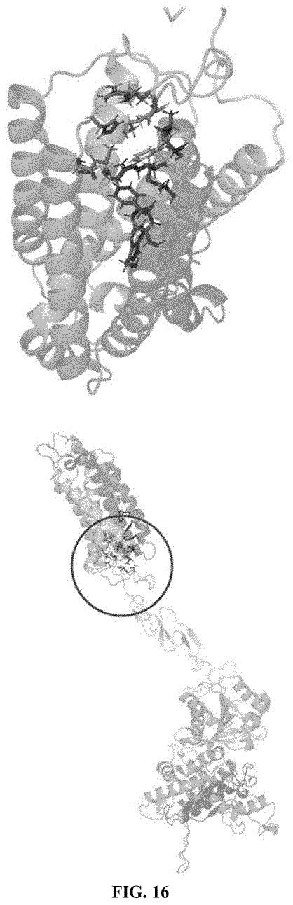

The target sequence having 872 residues used for building the model for mGluR2 is shown in FIG. 11. A hybrid model was generated in YASARA [see ref. 71] from the above sequence and the template structures with the PDB IDs, 1EWK [see ref. 72], 3LMK [see ref. 73] and 6N52 [see ref. 74]. These structures were obtained after a BLAST search against the PDB of the above mGluR2 sequence [see ref. 75]. YASARA generated 15 models initially from these structures and finally a hybrid model was generated using the best parts from these 15 initial models, to increase the accuracy beyond each contributor. FIG. 12 shows the hybrid model generated in YASARA with initial model in blue and hybridized parts in a different color. The resulting hybrid model obtained the following quality Z-scores (FIG. 13). This hybrid model was further validated by the following methods.

### MODFOLD Results

The model generated was validated using ModFOLD. [see ref. 76] The confidence and P-value for this model is HIGH: 1.161 E−3 with the global model quality score of 0.4399. In general, scores less than 0.2 indicate there may be incorrectly modelled domains and scores greater than 0.4 generally indicate more complete and confident models, which are highly similar to the native structure. Since the score for this model is higher than 0.4, it can be said with confidence that this model is a good one for mGluR2. The p-value represents the probability of each model being incorrect. The p-value for this model is 0.00116, meaning there is only a 1/1160 chance of this model being incorrect.

### Structure Analysis Verification Server (SAVES) Results

The second server used to validate this model was SAVES [see ref. 77-79] and its components, VERIFY 3D and ERRAT. VERIFY 3D analyses the residues based on their location and environment in the protein. It determines the compatibility of the model generated with its own amino acid sequence by assigning a structural class based on the location and environment and comparing the results to good structures. Verify 3D assigned a 3D-1D score of >0.2 for at least 86.72% of the amino acids. This implies that the model is compatible with its sequence.

The ERRAT server is another part of the SAVES database. It helps in verifying protein structures. The error values are plotted as a function of the position of a sliding 9-residue window. The function is based on the statistics of non-bonded atom-atom interactions in the structure. The plot for the hybrid model generated by YASARA for mGluR2 is shown below. Regions that can be rejected at 95% confidence level are yellow. 5% of a good protein structure are normally expected to have an error value above this level. Regions that can be rejected at 99% confidence interval are red. It can be seen from the figure below that the model contains significantly low red colored regions. The quality factor for this model is 96.52. Therefore, it is a good model according to ERRAT.

### SWISS Model-QMEAN Results [See Ref. 78]

QMEAN is a composite scoring function which derives both global and local absolute quality estimates based on one single model. The global scores are originally in a range [0,1] with one being good. Per default they are transformed into Z-scores to relate them with what we would expect from high resolution X-ray structures. The local scores are a linear combination of the 4 statistical potential terms as well as the agreement terms evaluated on a per residue basis. They are as well in the range [0,1] with one being good.

The QMEAN score is −2.04. Below is an image showing the sequence of the protein colored by local quality. The orange areas denote poor quality whereas blue ones are of good quality. It can be seen from the image that most of the residues forming a part of the alpha helices and beta. Sheets have high confidence that they are predicted accurately whereas the loops connecting them do not have good confidence scores for accuracy.

### Ramachandran Plot

Following is the Ramachandran plot for the model generated by YASARA for mGluR2. 89.1% (639) of the residues lie in the favored regions and 10.6% (76) lie in the additionally allowed regions. There are 0.3% (2) residues in the generously allowed regions and no residues in disallowed regions. This is further evidence of a quality model structure.

Docking of Known mGluR2 Binders

As further test of model quality, known binders were docked into the predicted binding site (vide infra). These known ligands were chosen from a list of PAMs previously reported [80]. The method used for docking known binders was similar to that of the PAMs in this study. In total 48 known ligands were used to dock into the binding site. The grid box generated for binding consisted of residues within 10 Å from the center of the predicted binding site. FIG. 15 shows docking scores for the top known ligands into the designated binding site.

### Prediction of Binding Site

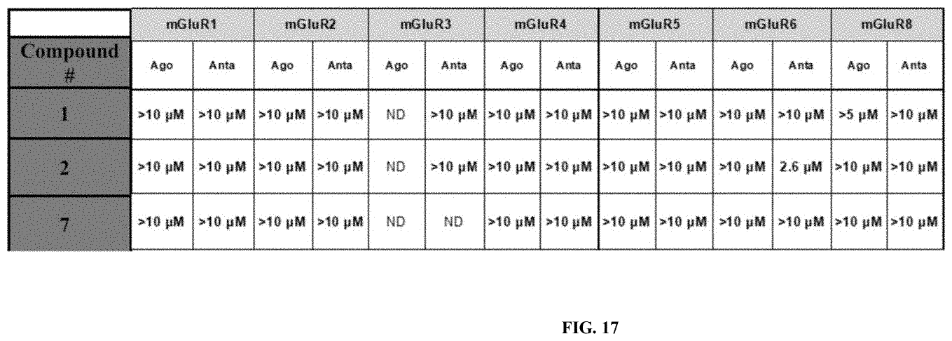

For binding site predictions, three servers were used, POOL [see ref. 81], DEPTH [see ref. 82], and MetaPocket [see ref. 83]. The consolidated results are as follows. The binding site identified is comprised of the following residues: Phe623, Arg635, Leu639, Thr709, Arg720, Cys721, His723, Asp725, Met728, Trp773, Phe776, Phe780, Arg788, Val789, Thr791, Met794. mGluR2 functional activity determined by GloSensor cAMP assay (see refs. 84, 8511: The functional assays of mGluR1-6 and 8 are shown in FIG. 17. In the figure: Ago: agonist activity; Anta: antagonist activity; The EC⁵⁰ value of agonist activity or the IC₅₀ value of antagonist activity >10 μM indicates that no curve was noted in the dose-response up to 10 μM; ND: Not determined.

### Example 1—Synthesis of 1-methyl-1H-imidazo[4,5-b]pyridine-2-carbaldehyde (The Intermediate

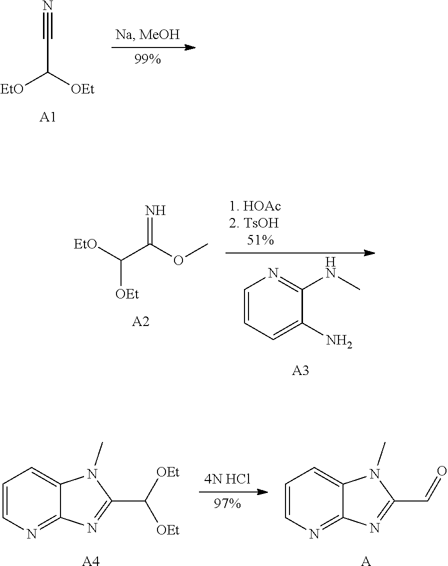

To a stirred solution of diethoxyacetonitrile (A1, 6.0 g, 46 mM) in absolute methanol (15 mL) under an atmosphere of nitrogen was added a solution of sodium methylate that generated by adding sodium (0.25 g, 4.7 mM) in absolute methanol (2 mL). The mixture was stirred at room temperature until the nitrile disappeared completely in 4 h, and was then treated with solid carbon dioxide to form sodium carbonate precipitates. The sodium carbonate was filtered off and washed with methanol. The filtrate was evaporated under vacuum on rotary evaporator at a bath temperature less than 30° C. The resulting liquid was dissolved in ether and filtered to remove the remaining salts and evaporated to give the methyl 2,2-diethoxyethanimidoate (A2) that was used directly in next step.

N²-methylpyridine-2,3-diamine (A3, 1.1 g, 8.9 mM) was dissolved in 1,2-dimethoxyethane (30 ml). A2 (3.1 g, 19 mM) and glacial acetic acid (1 ml) were added to the solution under stirring. The reaction mixture was stirred at room temperature for 3 h, and was refluxed for 5 h. p-Toluenesulfonic acid monohydrate (cat.) was then added to the reaction mixture, which was refluxed for 7 h. The mixture was concentrated under reduced pressure, and the residue was diluted with toluene (30 mL). The mixture was refluxed for 7 h, cooled, and mixed with a solution of sodium carbonate (2 g) in water (50 mL). The crude product was extracted with ethyl acetate. The combined extract was dried over magnesium sulfate and concentrated under reduced pressure to give a dark brown solid, which was purified by silica-gel column chromatography to obtain 2-(diethoxymethyl)-1-methyl-1H-imidazo[4,5-b]pyridine (A4, 0.83 g, 3.5 mM) in 39.5% yield. A4 (0.83 g, 3.5 mM) was mixed under vigorous stirring with 4N Hydrochloric acid solution (5 ml). The reaction mixture was stirred at 58° C. for 3 h and then was evaporated to dryness. The residue was mixed with dioxane (15 mL), and concentrated under reduced pressure to remove residual water. The crude product was purified by silica-gel column chromatography to give 1-methyl-1H-imidazo[4,5-b]pyridine-2-carbaldehyde (A, 0.55 g, 3.4 mM) in 97% yield.

### Example 2—Synthesis of 5,6-dihydro-4H-imidazo[4,5, 1-ij][1, 7]naphthyridine-2-carbaldehyde (THE intermediate B)

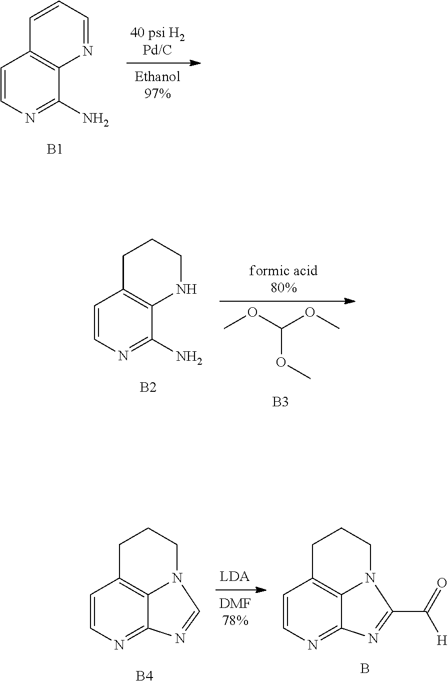

A 500 mL high-pressure Parr vessel was charged with 1,7-naphthyridin-8-amine (B1, 1.0 g 6.9 mM)Pd(OH)₂/C (20%, 0.5 g), conc.hydrochloric acid (37%) and ethanol (20 mL). The mixture was hydrogenated in Parr apparatus at 40 psi for five days. The LC-MS result indicated that the reaction was completed. The mixture was filtered through celite and evaporated to give yellow residue of hydrochloric salt. This residue was dissolved in water (20 mL), basified to pH 12 with 1N sodium hydroxide solution, and extracted with dichloromethane (4×20 mL). The extract was dried over sodium sulfate and evaporated to give 1,2,3,4-tetrahydro-1,7-naphthyridin-8-amine (B2, 1.0 g, 6.70 mM) as a tan solid in 97% yield. A high-pressure vessel, equipped with a magnetic stirring bar, was charged with B2, trimethylorthoformate (B3), and formic acid (cat.). The mixture was heated at 100° C. for 14 h, and the mixture was then evaporated under vacuum and the dark residue was mixed with 10 mL of saturated sodium carbonate solution. The mixture was extracted with dichloromethane three times. The extract was dried over sodium sulfate and the solvent was evaporated. The crude dark oil was purified by silica-gel column chromatography to give 5,6-dihydro-4H-imidazo[4,5,1-ij][1,7]naphthyridine (B4, 0.85 g, 5.34 mM) in 80% yield. A 100 mL 3-neck round bottomed flask, quipped with a magnetic stirring bar, nitrogen gas inlet, thermometer and a septum, was charged with B4 (1.2 g, 7.5 mM) and anhydrous tetrahydrofuran (20 mL). The suspension was heated to completely dissolve the material, and then cooled to room temperature to form fine suspension. This suspension was cooled to −75° C. and lithium diisopropylamide (5.6 mL, 2M in tetrahydrofuran) was added dropwise to keep temperature below −60° C. The mixture was stirred at −70° C. for 3 h, and then anhydrous N,N-dimethylmethanamide (1.0 g, 15.1 mM) was added over 5 min at temperature below −60° C. The result mixture was slowly warmed to room temperature, and then stirred at room temperature for 12 h. The reaction solution was cooled with an ice bath. Saturated aqueous solution of disodium hydrogen phosphate was added until pH 8.0-8.5. The mixture was extracted with dichloromethane and the concentrated residues was purified by silica-gel column chromatography to give 5,6-dihydro-4H-imidazo[4,5,1-ij][1,7]naphthyridine-2-carbaldehyde (8, 1.1 g, 5.88 mM) in 78% yield.

### Example 3—Synthesis of 3-(cyclopropylmethyl)-8-(trifluoromethyl)-[1,2,4]triazolo[4,3-a]pyridine-7-carbaldehyde (The Intermediate C)

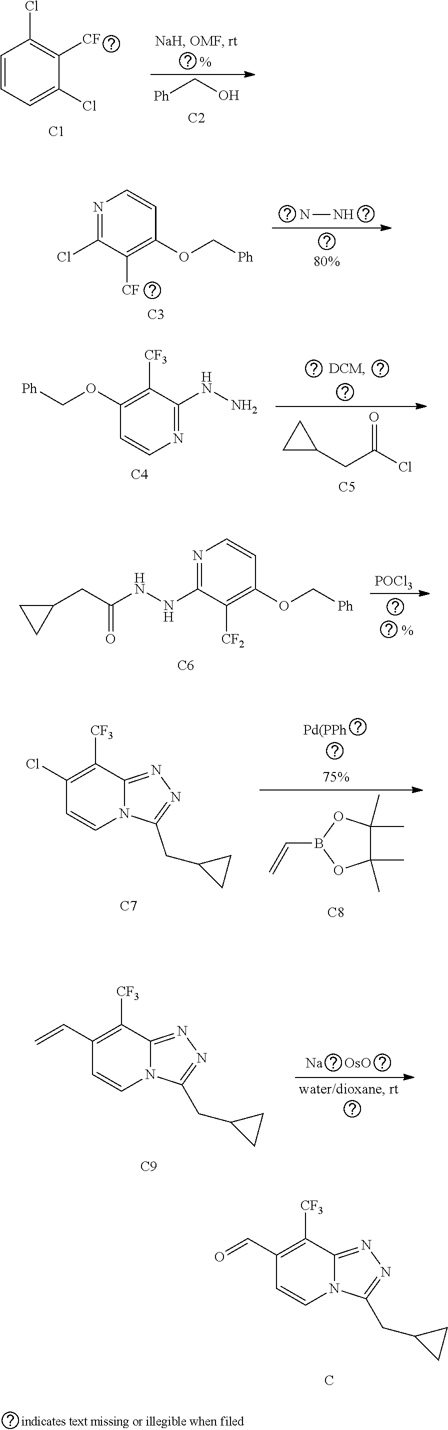

A solution of benzyl alcohol (2.4 g, 22 mM) was added to a mixture of 60% sodium hydride dispersion in mineral oil (0.97 mg, 24 mM) and dimethylformamide (80 mL) in an ice bath at 0° C. The mixture was allowed to stir at 0° C. for 30 min, and then a solution of 2,4-dichloro-3-(trifluoromethyl)pyridine (C, 5.0 g, 23 mM) in dimethylformamide (10 mL) was quickly added. The resulting mixture was stirred at 0° C. for 1 h, then quenched by the addition of water. The aqueous mixture was extracted with ethyl acetate. The combined organic layers were washed with brine, dried over magnesium sulfate, filtered, and concentrated in vacuo. The residue was purified by silica-gel column chromatography (5:1 hexanes/ethyl acetate) to afford 4-(benzyloxy)-2-chloro-3-(trifluoromethyl)pyridine (C3, 1.9 g, 6.9 mM) in 30% yield. To a suspension of C3 (1.09 g, 3.79 mM) in 1,4-dioxane (9 mL), was added hydrazine monohydrate (3.68 mL, 75.8 mM). The reaction mixture was heated at 160° C. for 30 min under microwave irradiation. After the mixture was cooled to room temperature, the volatiles were evaporated in vacuo. The residue thus obtained was dissolved in dichloromethane and washed with saturated sodium bicarbonate aqueous solution. The organic layer was separated, dried over sodium sulfate, concentrated in vacuo and purified by silica-gel column chromatography to give 4-(benzyloxy)-2-hydrazineyl-3-(trifluoromethyl)pyridine (C4, 0.86 g, 3.03 mM) in 80% yield.

To a cooled solution of C4 (0.5 g, 1.77 mM) and triethylamine (0.27 g, 2.65 mM) in anhydrous dichloromethane (10 mL) at 0° C. was added cyclopropylacetyl chloride (C5, 0.25 g, 2.12 mM). The reaction mixture was stirred at room temperature for 16 h. Saturated sodium bicarbonate aqueous solution was then added. The resulting solution was extracted with dichloromethane. Combined organic layer was dried under sodium sulfate, concentrated in vacuo and purified by silica-gel column chromatography to give N′-(4-(benzyloxy)-3-(trifluoromethyl)pyridin-2-yl)-2-cyclopropylacetohydrazide (C6, 0.53 g, 1.45 mM) in 82% yield. A mixture of C6 (1.0 g, 2.74 mM) and phosphorus oxychloride (0.5 mL, 5.47 mM) in 1,2-dichloroethane (10 ml) was heated at 150° C. under microwave irradiation for 10 min. After cooling to room temperature, the resulting reaction mixture was diluted with dichloromethane and washed with saturated sodium bicarbonate aqueous solution. The organic layer was separated, dried over sodium sulfate, and the volatiles were evaporated in vacuo. The residue thus obtained was purified by silica-gel column chromatography to give 7-chloro-3-(cyclopropylmethyl)-8-(trifluoromethyl)[1,2,4]triazolo[4,3-a]pyridine (C7, 0.38 g, 1.37 mM) in 50% yield.

A suspension of C7 (0.7 g, 2.54 mM), 4,4,5,5-tetramethyl-2-vinyl-1,3,2-dioxaborolane (C8, 0.65 mL, 3.81 mM), tetrakis(triphenylphosphine)palladium (0.29 g, 0.25 mM), and sodium bicarbonate (saturated aqueous solution, 2.0 mL) in 1,4-dioxane (10 mL) was heated at 150° C. under microwave irradiation for 18 min. After cooling, the resulting reaction mixture was diluted with ethyl acetate and water and filtered through a pad of diatomaceous earth. The filtrate was washed with water and brine and extracted with ethyl acetate. The organic layer was separated, dried with sodium sulfate, and concentrated in vacuo. The residue was purified by silica-gel column chromatography to give 3-(cyclopropylmethyl)-8-(trifluoromethyl)-7-vinyl-[1,2,4]triazolo[4,3-a]pyridine (C9, 0.51 g, 1.85 mM) in 73% yield.

A solution of C9 (0.5 g, 1.87 mM), sodium periodate (1.2 g, 5.6 mM), osmium tetroxide (2.5% in tert-butanol, 0.95 ml, 0.093 mM) in water (5 mL) and 1,4-dioxane (20 ml) was stirred at room temperature for 2 h. The resulting reaction mixture was diluted with ethyl acetate and water, and filtered through a pad of diatomaceous earth. The filtrate was extracted with ethyl acetate. The organic layer was separated, dried over sodium sulfate, and concentrated in vacuo. The solid residue purified by silica-gel column chromatography to give 3-(cyclopropylmethyl)-8-(trifluoromethyl)[1,2,4]triazolo[4,3-a]pyridine-7-carbaldehyde (C, 0.33 g, 1.22 mM) in 65% yield

### Example 4—Synthesis of 4-(2-methoxy-4-(trifluoromethyl)phenyl)piperidine (The Intermediate D)

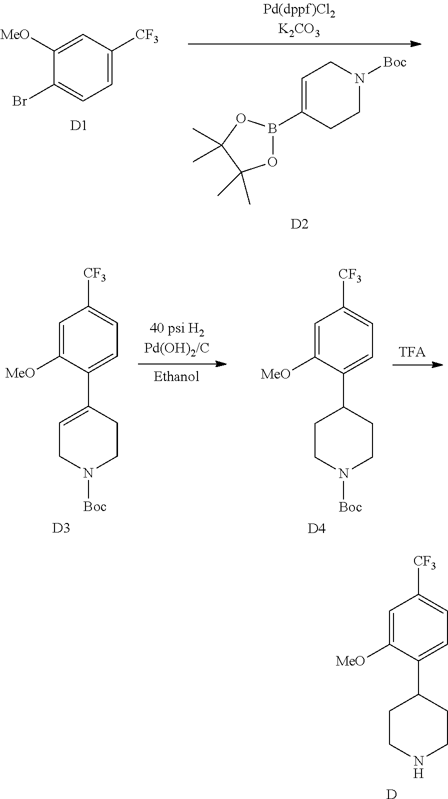

To a mixture of tert-butyl 4-(4,4,5,5-tetramethyl-1,3,2-dioxaborolan-2-yl)-5,6-dihydropyridine-1 (2H)-carboxylate (D2, 1.33 g, 4.3 mM) and potassium carbonate (2.2 g, 15.7 mM) in dioxane with water were added (1, 1-bis(diphenylphosphino)ferrocene)-dichloropalladium (143 mg, 0.2 mM) and 1-bromo-2-methoxy-4-(trifluoromethyl)benzene (D1, 1.0 g, 3.9 mM). The reaction was heated to 80° C. overnight under nitrogen. The resulting black mixture was cooled to room temperature, diluted with water and extracted with ethyl acetate. The combined organic layers were dried with sodium sulfate, filtered, and concentrated under reduced pressure. The crude black oil was purified by silica-gel column chromatography to get tert-butyl-4-(2-methoxy-4-(trifluoromethyl)phenyl)-3,6-dihydropyridine-1(2H)-carboxylate (D3, 1.2 g, 3.35 mM) in 86% yield. A 500 mL high pressure Parr vessel was charged with D3 (1.2 g, 3.4 mM), Pd(OH)₂/C (20%, 0.5 g), ethanol (50 mL). The mixture was hydrogenated in Parr apparatus at 40 psi for 4 h. The LCMS showed the reaction was completed. The mixture was filtered through celite and evaporated to give a yellow residue of hydrochloride salt. This residue was dissolved in water (20 mL), and extracted with dichloromethane (4×20 mL). The extract was dried over sodium sulfate and evaporated to give tert-butyl-4-(2-methoxy-4-(trifluoromethyl)phenyl)piperidine-1-carboxylate (D4, 1.2 g, 3.4 mM) in 99% yield. To a solution of D4 (1.2 g, 3.4 mM) in dichloromethane (5 mL) was added trifluoroacetic acid (1 mL). The mixture was stirred at room temperature for 2 h. The solvents was removed under reduced pressure to give 4-(2-methoxy-4-(trifluoromethyl)phenyl)piperidine (D, 0.86 g, 3.4 mM) in 99% yield.

### Example 5—Synthesis of 3-fluoro-2-(piperidin-4-yl)-5-(trifluoromethyl)pyridine (The Intermediate E)

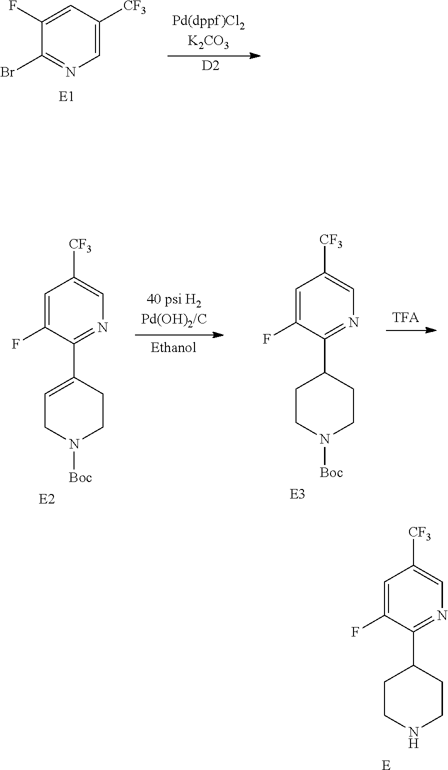

To a mixture of D2 (2.3 g, 16.4 mM) in dioxane and water were added (1,1-bis(diphenylphosphino)ferrocene)-dichloropalladium (150 mg, 0.2 mM) and 2-bromo-3-fluoro-5-(trifluoromethyl)pyridine (E1, 1.0 g, 4.1 mM). The reaction was heated to 80° C. overnight under nitrogen. The resulting black mixture was cooled to room temperature, diluted with water and extracted with ethyl acetate. The combined organic layer was dried over sodium sulfate, filtered, and concentrated under reduced pressure. The crude black oil was purified by silica-gel column chromatography to get tert-butyl-3-fluoro-5-(trifluoromethyl)-3′,6′-dihydro-[2,4′-bipyridine]-1′(2′H)carboxylate (E2, 1.2 g, 3.4 mM) in 84% yield. A 500 mL high pressure Parr vessel was charged with E2 (1.2 g, 3.46 mM), Pd(OH)₂/C (20%, 0.5 g), ethanol (50 mL). The mixture was hydrogenated in Parr apparatus at 40 psi for 4 h. The LCMS showed that the reaction was completed. The mixture was filtered through celite and evaporated to give yellow residue of hydrochloride salt. This residue was dissolved in water (20 ml), and extracted with dichloromethane (4×20 mL). The extract was dried over sodium sulfate and evaporated to give tert-butyl-4-(3-fluoro-5-(trifluoromethyl)pyridin-2-yl)piperidine-1-carboxylate (E3, 1.2 g, 3.42 mM) in 99% yield.

To a solution of E3 (1.2 g, 3.4 mM) in dichloromethane (5 ml) was added trifluoroacetic acid (1 mL). The mixture was stirred at room temperature for 2 h. The solvents was removed under reduced pressure to give 3-fluoro-2-(piperidin-4-yl)-5-(trifluoromethyl)pyridine (E, 0.85 g, 3.4 mM) in 99% yield.

### Example 6—Synthesis of 4-(2-fluoro-4-(trifluoromethyl)phenyl)-3-methylpiperidine (The Intermediate F)

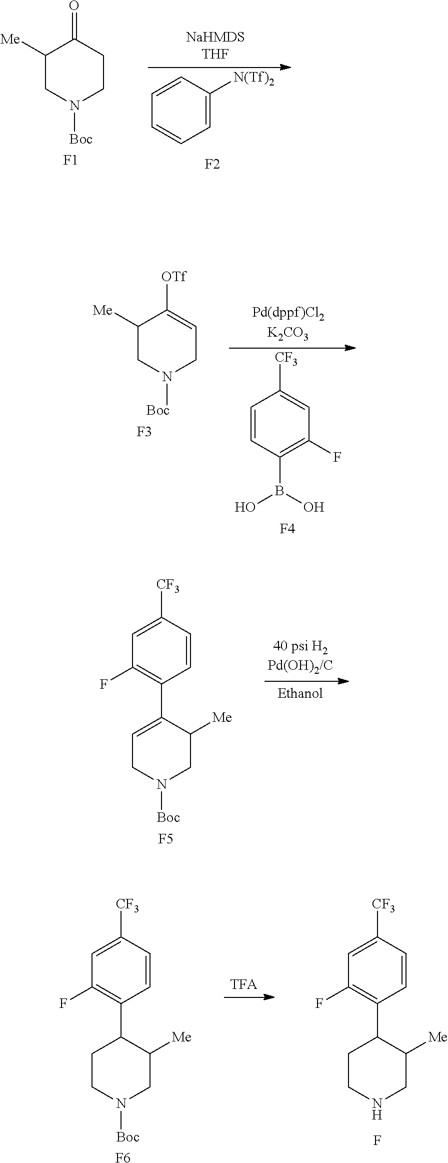

A 100 mL 3-neck round bottomed flask quipped with a magnetic stirring bar, nitrogen gas inlet, thermometer and a septum was charged with terl-butyl-3-methyl-4-oxopiperidine-1-carboxylate (F1, 1.0 g, 4.7 mM). After cooling this solution to −78° C., a solution of sodium bis(trimethylsilyl)amide (5.6 mL, 1.0 M in tetrahydrofuran) was added dropwise, maintaining the reaction temperature below −70° C. After the addition was complete, the reaction mixture was stirred at −78° C. for 1 h, and then a solution of 1,1,1-trifluoro-N-phenyl-N-((trifluoromethyl)sulfonyl)methanesulfonamide (F2, 2.0 g, 5.6 mM) in tetrahydrofuran was added over 10 min. The solution was allowed to warm to 0° C. and was then stirred for 2 h. The reaction mixture was concentrated under reduced pressure and the residue was subjected to silica gel chromatography to give terl-butyl-3-methyl-4-(((trifluoromethyl)sulfonyl)oxy)-3,6-dihydropyridine-1(2H)-carboxylate (F3, 1.2 g, 3.5 mM) in 74% yield. To a mixture of (2-fluoro-4-(trifluoromethyl)phenyl)boronic acid (F4, 0.5 g, 2.4 mM) and potassium carbonate (1.33 g, 9.6 mM) in dioxane and water were added (1,1-bis(diphenylphosphino)ferrocene)-dichloropalladium (88 mg, 0.12 mM) and F3 (0.83 g, 2.4 mM). The reaction was heated to 80° C. overnight under nitrogen. The resulting black mixture was cooled to room temperature, diluted with water and extracted with ethyl acetate. The combined organic layers were dried with sodium sulfate, filtered, and concentrated under reduced pressure. The crude black oil was purified by silica-gel column chromatography to get terl-butyl-4-(2-fluoro-4-(trifluoromethyl)phenyl)-3-methyl-3,6-dihydropyridine-1(2H)-carboxylate (F5, 0.62 g, 1.7 mM) in 71% yield. A 500 mL high-pressure Parr vessel, was charged with F5 (0.62 g, 1.7 mM), Pd(OH)₂/C (20%, 0.2 g), ethanol (50 mL). The mixture was hydrogenated in Parr apparatus at 40 psi for 4 h. The LC-MS showed that the reaction was completed. The mixture was filtered through celite and evaporated to give yellow residue of hydrochloride salt. This residue was dissolved in water (20 mL), and extracted with dichloromethane (4×20 mL). The extract was dried over sodium sulfate and evaporated to give terl-butyl-4-(2-fluoro-4-(trifluoromethyl)phenyl)-3-methylpiperidine-1-carboxylate (F6, 0.62 g, 1.7 mM) in 99% yield. To a solution of F6 (0.62 g, 1.7 mM) in dichloromethane (5 ml) was added trifluoroacetic acid (1 ml). The mixture was stirred at room temperature for 2 h. Solvents was removed under reduced pressure to give 4-(2-fluoro-4-(trifluoromethyl)phenyl)-3-methylpiperidine (F, 0.44 g, 1.67 mM) in 98% yield.

### Example 7—Synthesis of 1-(3-fluoro-5-(trifluoromethyl)pyridin-2-yl)piperazine (The intermediate G)

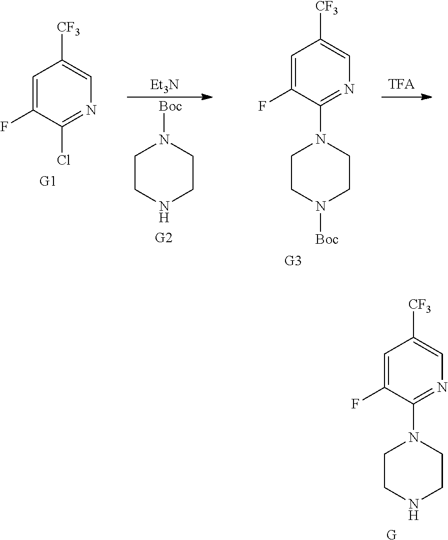

A 100 mL round bottomed flask was added 2-chloro-3-fluoro-5-(trifluoromethyl)pyridine (G1, 0.25 g, 1.25 mM), tert-butyl piperazine-1-carboxylate (G2, 0.7 g, 3.76 mM) and triethylamine (0.51 g, 5.0 mM) in anhydrous acetonitrile (20 mL). The reaction mixture was heated to refluxed for 2 h.

The resulting mixture was cooled to room temperature and purified by silica-gel column chromatography to give tert-butyl-4-(3-fluoro-5-(trifluoromethyl)pyridin-2-yl)piperazine-1-carboxylate (G3, 0.41 g, 1.17 mM) in 95% yield.

To a solution of G3 (0.41 g, 1.17 mM) in dichloromethane (5 mL) was added trifluoroacetic acid (1 mL). The mixture was stirred at room temperature for 2 h. The solvents was removed under reduced pressure to give 1-(3-fluoro-5-(trifluoromethyl)pyridin-2-yl)piperazine (G, 0.29 g, 1.16 mM) in 99% yield.

### Example 8—Synthesis of 1-(3-methoxy-5-(trifluoromethyl)pyridin-2-yl)piperazine (The Intermediate H)

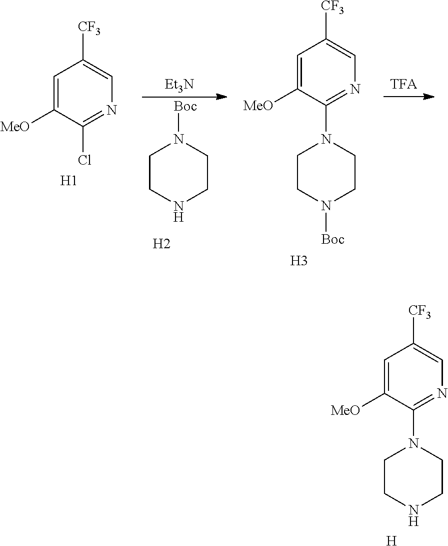

A 100 mL round bottomed flask was added 2-chloro-3-methoxy-5-(trifluoromethyl)pyridine (H1, 0.25 g, 1.18 mM), tert-butyl-piperazine-1-carboxylate (H2, 0.66 g, 3.54 mM) and triethylamine (0.48 mg, 4.7 mM) in anhydrous acetonitrile (20 mL). The reaction mixture was heated to refluxed for 2 h. The resulting mixture was cooled to room temperature and purified by silica-gel column chromatography to give terl-butyl-4-(3-methoxy-5-(trifluoromethyl)pyridin-2-yl)piperazine-1-carboxylate (H3, 0.40 g, 1.11 mM) in 94% yield.

To a solution of H3 (0.40 g, 1.11 mM) in dichloromethane (5 mL) was added trifluoroacetic acid (1 mL). The mixture was stirred at room temperature for 2 h. Solvents was removed under reduced pressure to give 1-(3-methoxy-5-(trifluoromethyl)pyridin-2-yl)piperazine (H, 0.28 g, 1.08 mM) in 97% yield.

### Example 9—Synthesis of (1R, 5S, 6R)-6-((2-methoxy-4-(trifluoromethyl)phenoxy)methyl)-3-azabicyc/o[3. 1.O]hexane (The Intermediate /)

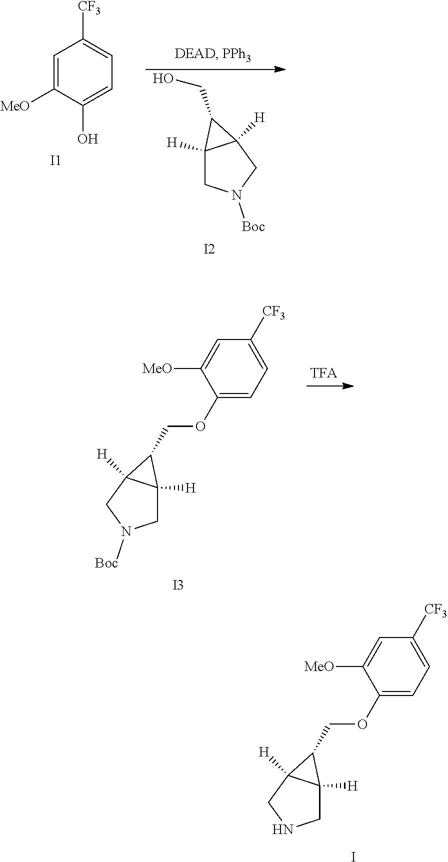

To (1R, 5S, 6R)-tert-butyl 6-(hydroxymethyl)-3-azabicyclo[3.1.0]hexane-3-carboxylate (12, 0.5 g, 2.3 mM) under nitrogen was added 2-methoxy-4-(trifluoromethyl)phenol (11, 0.45 g, 2.3 mM) and triphenyl phosphine (0.9 g, 3.5 mM) in THF. Diethyl azodicarboxylate solution (40 wt. % in toluene, 1.5 g, 3.5 mM) was added and the reaction was stirred under nitrogen for 16 h. The reaction was stripped in vacuo to give orange oil. The crude product was purified via flash chromatography to give tert-butyl-(1R,5S,6r)-6-((2-methoxy-4-(trifluoromethyl)phenoxy)methyl)-3-azabicyclo[3.1.0]hexane-3-carboxylate (13, 0.44 g, 1.14 mM) in 48% yield.

To a solution of 13 (0.44 g, 1.14 mM) in dichloromethane (5 mL) was added trifluoroacetic acid (1 mL). The mixture was stirred at room temperature for 2 h. The solvents was removed under reduced pressure to give (1R, 5S, 6R)-6-((2-methoxy-4-(trifluoromethyl)phenoxy)methyl)-3-azabicyclo[3.1.0]hexane (1, 0.3 g, 1.05 mM) in 92% yield.

### Example 10—Synthesis of (1R, 5, r)-6-(((3-fluoro-5-(trifluoromethyl)pyridin-2-yl)oxy)methyl)-3-azabicyclo[3.1.0]hexane (The intermediate J)

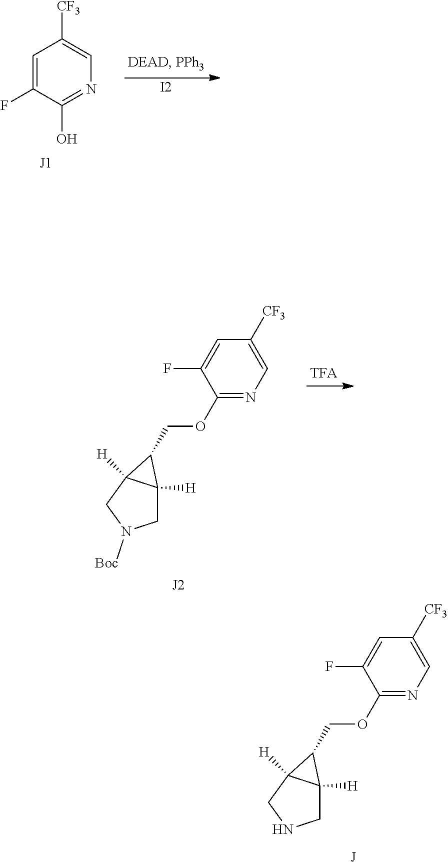

To 12 (0.64 g, 3.0 mM) under nitrogen was added 2-methoxy-4-(trifuoromethyl)phenol (J1, 0.55 g, 3.0 mM) and triphenylphosphine (1.2 g, 4.5 mM) in THF. Diethyl azodicarboxylate solution (40 wt. % in toluene, 1.97 g, 4.5 mM) was added and the reaction was stirred under nitrogen for 16 h. The reaction was stripped in vacuo to give an orange oil. The crude product was purified via flash chromatography to give tert-butyl-(1R,5S,6r)-6-(((3-fluoro-5-(trifluoromethyl)pyridin-2-yl)oxy)methyl)-3-azabicyclo[3.1.0]hexane-3-carboxylate (J2, 0.6 g, 1.59 mM) in 52% yield.

To a solution of J2 (0.6 g, 1.59 mM) in dichloromethane (5 mL) was added trifluoroacetic acid (1 mL). The mixture was stirred at room temperature for 2 h. The solvents was removed under reduced pressure to give (1R, 5S, 6r)-6-(((3-fluoro-5-(trifuoromethyl)pyridin-2-yl)oxy)methyl)-3-azabicyclo[3.1.0]hexane (J, 0.4 g, 1.45 mM) in 91% yield.

### Example 11—Synthesis of (1R, 5, 6R)-6-(((3-methoxy-5-(trifluoromethyl)pyridin-2-yl)oxy)methyl)-3-azabicyc/o[3. 1.O]hexane (The Intermediate K)

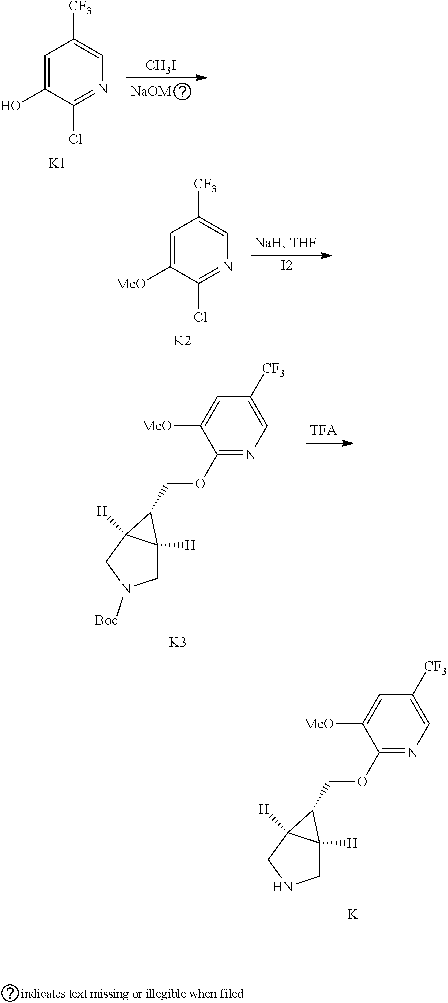

To a solution of 2-chloro-5-(trifluoromethyl)pyridin-3-ol (K1, 0.50 g, 2.53 mM) in dry Dimethylformamide (16 mL) under nitrogen was added sodium methoxide (0.14 g, 2.53 mM) in one portion. After the mixture was stirred for 4 h, methyl iodide (0.36 g, 2.53 mM) was added and stirred for 16 h. At the end of the reaction, the mixture was diluted with water (20 mL) and then extracted with ethyl acetate. The combined organic layers were dried over magnesium sulfate and then concentrated under reduced pressure on a rotary evaporator. The resulting residue was purified by silica-gel flash chromatography with ethyl acetate/hexane (1:5) as eluents to afford 2-chloro-3-methoxy-5-(trifluoromethyl)pyridine (K2, 0.45 g, 2.12 mM) in 84% yield. To a solution of K2 (0.2 g, 0.95 mM) in tetrahydrofuran (10 mL) was added under nitrogen 12 (0.2 g, 0.95 mM) and sodium hydride (68 mg, 2.84 mM). The mixture was stirred at room temperature for 24 h. At the end of the reaction, the solvents were removed on rotary evaporator. The resulting residue was purified by silica-gel column chromatography to give tert-butyl (1R,5S,6R)-6-(((3-methoxy-5-(trifluoromethyl)pyridin-2-yl)oxy)methyl)-3-azabicyclo[3.1.0]hexane-3-carboxylate (K3, 0.21 g, 0.54 mM) in 57% yield. To a solution of K3 (0.21 g, 0.54 mM) in dichloromethane (5 mL) was added trifluoroacetic acid (1 mL). The mixture was stirred at room temperature for 2 h. The solvents was removed under reduced pressure to give (1R, 5S, 6R)-6-(((3-methoxy-5-(trifluoromethyl)pyridin-2-yl)oxy)methyl)-3-azabicyclo[3.1.0]hexane (K, 0.15 g, 0.52 mM) in 96% yield.

### Example 12—Synthesis of 4-(2-(2-fluoroethoxy)-4-(trifluoromethyl)phenyl)piperidine (The Intermediate L)

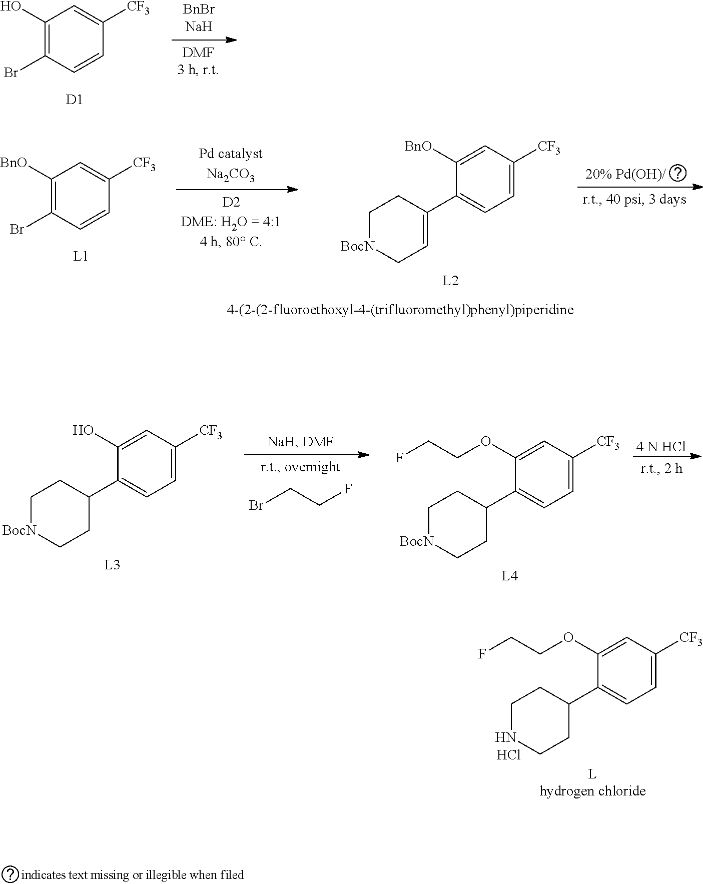

To a mixture of 2-bromo-5-(trifluoromethyl)phenol (D1, 2.41 g, 10 mM) in DMF (50 mL) were added benzyl bromide (1.56 mL, 13 mM). The reaction was stirred for 3 h under room temperature before it was diluted with water and extracted with ethyl acetate. The combined organics were dried over sodium sulfate, filtered, and dried under reduced pressure. The crude product was purified with silica gel column to get 2-(benzyloxy)-1-bromo-4-(trifluoromethyl)benzene as a white solid (L1, 2.64 g, 80%). ¹H NMR (300 MHz, CDCl₃) δ ppm 5.20 (s, 2H), 7.13 (d, J=8.4 Hz, 1H), 7.17 (s, 1H), 7.36-7.51 (m, 5H), 7.69 (d, J=8.1 Hz, 1H). ¹³C NMR (75 MHz, CDCl₃) δ ppm 155.5, 135.8, 134.0, 130.9 (q, J=31.4 Hz), 128.7, 128.3, 127.2, 123.9 (q, J=271.5 Hz), 118.8, 116.7, 110.2, 71.0. LC-MS, calculated for C₁₄H₁₀BrF₃O: 329.99; observed: m/z 330.0, 332.0 [M+H]⁺. To a mixture of L1 (1.0 g, 3.0 mM) and sodium carbonate (0.65 g, 6.1 mM) in DMF/water (4:1, 25 mL) were added [1,1′-bis(diphenylphosphino)ferrocene]dichloropalladium (II) (0.2 g, 0.27 mM) and D2 (0.95 g, 3.1 mM). The reaction was heated to 80° C. for 4 h under nitrogen. The resulting black mixture was cooled to room temperature, diluted with water and extracted with ethyl acetate. The combined organics were dried over sodium sulfate, filtered, and dried under reduced pressure. The crude black oil was purified with silica gel column to get tert-butyl-4-(2-(benzyloxy)-4-(trifluoromethyl)phenyl)-3,6-dihydropyridine-1 (2H)-carboxylate as colorless oil (L2, 1.2 g, 92%). LCMS, calculated for C24H25F3NQ3: 433.19; observed: m/z 434.20 [M+H]+. A 500 mL high-pressure Parr vessel was charged with L2 (1.2 g, 2.76 mM), Pd(OH)₂/C (20%, 0.12 g) and ethanol (20 ml). The mixture was sealed and hydrogenated in Parr apparatus at 40 psi for 4 h. The LC-MS showed that the reaction was completed. The mixture was filtered through celite and evaporated to give tert-butyl-4-(2-hydroxy-4-(trifluoromethyl)phenyl)piperidine-1-carboxylate as waxy white solid (L3, 0.9 g, 95%). ¹H NMR (300 MHz, DMSO-d₆) δ ppm 1.40 (s, 9H), 1.48-1.50 (m, 2H), 1.68-1.72 (m, 2H), 2.70-2.90 (m, 2H), 3.00-3.08 (m, 1H), 4.05-4.09 (m, 2H), 6.90-7.10 (m, 2H), 7.28-7.31 (m, 1H), 10.1 (bs, 1H). ¹³C NMR (75 MHz, DMSO-d6) δ ppm 155.5, 154.3, 136.8, 128.1, 127.7, 124.7 (q, J=271.6 Hz), 116.0, 111.5, 79.0, 35.5, 31.4, 28.6: LC-MS, calculated for C₁₇H₂₂F₃NQ₃: 345.16; observed: m/z 346.2 [M+H]+. To an ice cooled solution of L3 (0.1 g, 0.3 mM) and 1-bromo-2-fluoroethane in DMF (1.5 mL) was added sodium hydride (10 mg, 0.4 mM). The reaction was warmed to room temperature and stirred overnight. The resulting mixture was quenched with water and extracted with ethyl acetate. The combined organics were dried over sodium sulfate, filtered, and dried under reduced pressure. The crude product was purified with silica gel column to get tert-butyl-4-(2-(2-fluoroethoxy)-4-(trifluoromethyl)phenyl)piperidine-1-carboxylate as colorless oil (L4, 57.0 mg, 49%). ¹H NMR (300 MHz, CDCl₃) δ ppm 1.48 (s, 9H), 1.52-1.65 (m, 2H), 1.80-1.85 (m, 2H), 2.79-2.88 (m, 2H), 3.11-3.21 (m, 1H), 4.21-4.33 (m, 4H), 4.69-4.72 (m, 1H), 4.85-4.88 (m, 1H), 7.04 (s, 1H), 7.24-7.28 (m, 2H). ¹³C NMR (75 MHz, CDCl₃) δ ppm 155.7, 154.9, 138.4, 129.4, 127.2, 124.0 (q, J=271.9 Hz), 118.2, 108.2, 81.6 (q, J=171.9 Hz), 79.4, 67.7, 44.4, 35.6, 31.5, 28.5. LC-MS, calculated for C₁₉H₂sF₄NQ₃: 391.18; observed: m/z 392.2 [M+H]+. To the mixture of L4 (40 mg, 0.1 mM) in dichloromethane (0.5 mL) was added a solution of 4N HCl in dioxane (0.4 mL). The reaction was stirred at room temperature for 20 min. The solvent was evaporated under vacuum to give the crude product L (4-(2-(2-fluoroethoxy)-4-(trifluoromethyl)phenyl)piperidine as a white solid, which was used without purification.

### Example 13—Synthesis of 3-methoxy-2-(piperidin-4-yl)-5-(trifluoromethyl)pyridine (The Intermediate M)

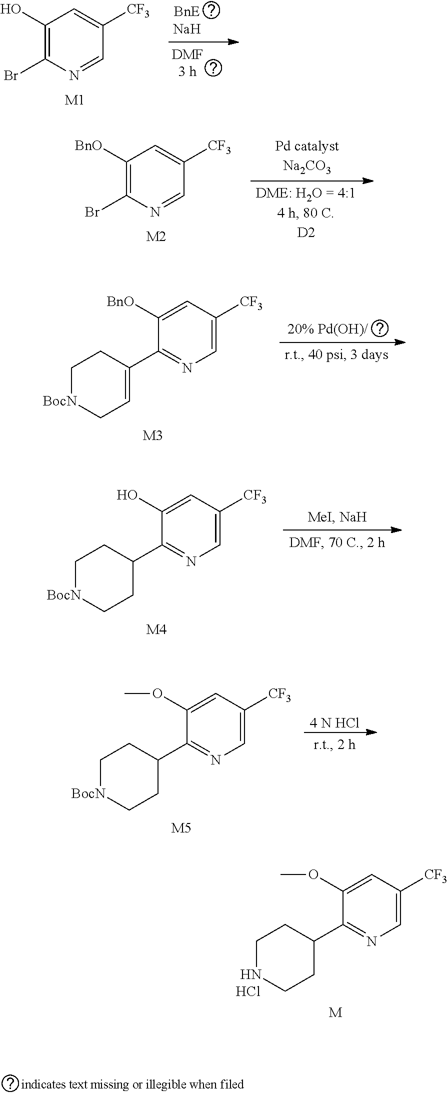

In a similar procedure as described for the synthesis of L 1, 2-(benzyloxy)-1-bromo-4-(trifluoromethyl)benzene (M2) was obtained as a white solid in 54% yield. LCMS: m/z 331.0, 333.0 [M+H]+, ¹H NMR (300 MHz, CDCl₃) δ ppm 5.25 (s, 2H), 7.28-7.48 (m, 6H), 8.30 (s, 1H). ¹³C NMR (75 MHz, CDCl3) δ ppm 152.1, 138.0, 134.6, 128.9, 128.7, 127.2, 127.1, 126.9, 121.4 (q, J=271.3 Hz), 116.4, 71.4. LC-MS, calculated for C₁₃H₉₈rF₃NQ: 330.98; observed: m/z 331.0, 333.0 [M+H]+. In a similar procedure as described for the synthesis of L2, tert-butyl-4-(2-(benzyloxy)-4-(trifluoromethyl)phenyl)-3,6-dihydropyridine-1 (2H)-carboxylate (M3) was obtained as a pale yellow oil in 64% yield. ¹H NMR (300 MHz, CDCl₃) δ ppm 1.49 (s, 9H), 2.50-2.69 (m, 2H), 3.3-3.62 (m, 2H), 4.05-4.30 (m, 2H), 5.08-5.20 (m, 2H), 6.48-6.70 (m, 1H), 7.25-7.45 (m, 6H), 8.45 (s, 1H). ¹³C NMR (75 MHz, CDCl₃) 5 ppm 154.9, 152.3, 137.5, 135.2, 128.9, 128.6, 127.4, 127.3, 127.1, 123.4 (q, J=272.9 Hz), 116.1, 79.7, 70.9, 53.4, 29.7, 28.5, 28.3, 28.2, 27.5. LC-MS, calculated for C₂₃H₂sF₃N₂O₃: 434.18; observed: m/z 435.1 [M+H]+. In a similar procedure as described for the synthesis of L3, tert-butyl-4-(2-hydroxy-4-(trifluoromethyl)phenyl)piperidine-1-carboxylate (M4) was obtained as a yellow solid in 80% yield. ¹H NMR (300 MHz, DMSO-d6) δ ppm 1.40 (s, 9H), 1.56-1.72 (m, 4H), 2.75-2.85 (m, 2H), 3.27-3.30 (m, 1H), 4.02-4.07 (m, 2H), 7.36 (s, 1H), 8.33 (s, 1H), 10.7 (bs, 1H). ¹³C NMR (75 MHz, DMSO-d6) δ ppm 156.9, 154.3, 151.0, 135.9, 124.1 (q, J=165.4 Hz), 124.0, 117.8, 79.0, 37.2, 30.0, 28.6, 25.0. LCMS, calculated for C₁₆H₂₁F3N₂O₃: 346.15; observed: m/z 347.2 [M+H]+. To a solution of M4 (100 mg, 0.3 mM) in DMF (1.2 mL) was added potassium carbonate (100 mg, 0.7 mM) and methyl iodide (26.0 μL, 0.4 mM). The reaction was heated to 70° C. for 2 h. The resulting reaction mixture was cooled to room temperature, diluted with water and extracted with ethyl acetate. The combined organics were dried over sodium sulfate, filtered, and dried under reduced pressure. The crude yellow oil was then stirred in 4N HCl solution of dioxane for 2 h at room temperature. Solvent was removed under vacuum. The crude product 3-methoxy-2-(piperidin-4-yl)-5-(trifluoromethyl)pyridine (M) was obtained as waxy yellow solid, which was used in the next reductive coupling reaction without purification.

### General Procedure of the Reductive Amination Reaction for Syntheses of Compounds 1-16.

To a stirred solution of hydrochloride salt of the amine derivatives such as the intermediates D-K (0.54 mM) in 1,2-dichloroethane (5 ml) under nitrogen at room temperature was added trimethylamine (0.22 g, 2.16 mM), magnesium sulfate (0.65 g, 5.41 mM) and the aldehyde derivatives such as the intermediates A-C (0.81 mM). After the mixture was stirred for 30 min, sodium triacetoxyborohydride (0.17 g, 0.81 mM) was added. The reaction mixture was stirred at room temperature overnight and then diluted with dichloromethane. The organic phase was washed with water and brine. The aqueous phase was extracted with dichloromethane. Combined organic layer was dried over sodium sulfate. The solvent was removed at reduced pressure, and the residue was purified by flash chromatography to give the product.

### Example 14—Preparation of Compound 1

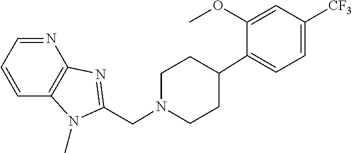 [SMILES: COc1cc(C(F)(F)F)ccc1C1CCN(Cc2nc3ncccc3n2C)CC1]

Compound 1. In a similar procedure of the reductive amination reaction as described afore, compound 1 was prepared by using the intermediates A and D. ¹H NMR (500 MHz, CD₃OD): δ 8.41 (d, 1H, J=5.0 Hz), 8.02 (d, 1H, J=8.5 Hz), 7.34-7.37 (m, 2H), 7.20 (d, 1H, J=7.5 Hz), 7.14 (s, 1H), 4.01 (s, 3H), 3.94 (s, 2H), 3.89 (s, 3H), 3.31-3.32 (m, 3H), 2.33-2.37 (m, 2H), 1.72-1.83 (m, 4H). LCMS, calculated for C21H23F3N4Q: 404.18; observed: m/z 405.15 [M+H]+.

### Example 15—Preparation of Compound 2

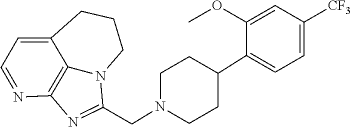 [SMILES: COc1cc(C(F)(F)F)ccc1C1CCN(Cc2nc3nccc4c3n2CCC4)CC1]

In a similar procedure of the reductive amination reaction as described afore, compound 2 was prepared by using the intermediates B and D. ¹H NMR (500 MHz, (CD₃)₂SO): δ 8.50 (d, 1H, J=5.0 Hz), 7.40 (d, 1H, J=8.5 Hz), 7.25 (d, 1H, J=8.5 Hz), 7.20 (s, 1H), 7.01 (d, 1H, J=4.5 Hz), 4.33 (t, 2H, J=6.0 Hz), 4.02 (s, 1H), 3.29 (s, 1H), 3.86 (s, 3H), 2.93-2.97 (m, 5H), 2.15-2.48 (m, 5H), 1.60-1.71 (m, 4H). LC-MS, calculated for C23H2sF3N4Q: 430.20; observed: m/z 431.20 [M+H]+.

### Example 16—Preparation of Compound 3 (JNJ-46356479)

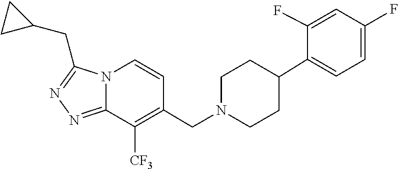 [SMILES: Fc1ccc(C2CCN(Cc3ccn4c(CC5CC5)nnc4c3C(F)(F)F)CC2)c(F)c1]

In a similar procedure of the reductive amination reaction as described afore, compound 3 was prepared by using the intermediates C and 1-(2,4-difluorophenyl)piperazine. ¹H NMR (500 MHz, CDCl₃) δ 8.23 (d, J=7.5 Hz, 1H), 7.45 (d, J=6.5 Hz, 1H), 6.90-6.91 (m, 1H), 6.78-6.81 (m, 2H), 3.81 (s, 2H), 3.10 (d, J=6.5 Hz, 2H), 3.01-3.09 (m, 4H), 2.63-2.75 (m, 4H), 1.21-1.26 (m, 1H), 0.60-0.62 (m, 2H), 0.33-0.34 (m, 2H). LC-MS, calculated for C22H22F5N5: 451.18; observed: m/z 452.05 [M+H]+.

### Example 17—Preparation of Compound 4

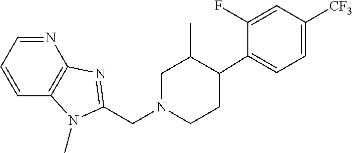 [SMILES: CC1CN(Cc2nc3ncccc3n2C)CCC1c1ccc(C(F)(F)F)cc1F]

In a similar procedure of the reductive amination reaction as described afore, compound 4 was prepared by using the intermediates A and F. ¹H NMR (500 MHz, CD3OD): δ 8.42 (d, 1H, J=4.5 Hz), 8.02 (d, 1H, J=8.5 Hz), 7.41-7.46 (m, 2H), 7.34-7.37 (m, 2H), 4.03 (s, 3H), 3.90 (s, 2H), 3.26-3.27 (m, 1H), 3.06-3.07 (m, 1H), 2.82 (d, 1H, J=10.5 Hz), 2.57 (d, 1H, J=10.5 Hz), 2.30-2.34 (m, 2H), 2.19 (m, 1H), 1.59 (m, 1H), 0.81 (d, 3H, J=7.0 Hz). LC-MS, calculated for C21H22F4N4: 406.18; observed: m/z 407.15 [M+H]+.

### Example 18—Preparation of Compound 5

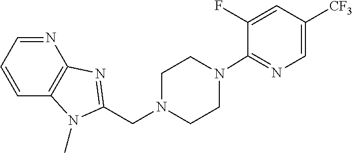 [SMILES: Cn1c(CN2CCN(c3ncc(C(F)(F)F)cc3F)CC2)nc2ncccc21]

In a similar procedure of the reductive amination reaction as described afore, compound 5 was prepared by using the intermediates A and E. ¹H NMR (500 MHz, CDCl₃): δ 8.54 (d, 1H, J=4.5 Hz), 8.22 (s, 1H), 7.67 (d, 1H, J=8.5 Hz), 7.38 (d, 1H, J=13.0 Hz), 7.20-7.23 (m, 1H), 3.93 (s, 5H), 3.66 (t, 4H, J=4.5 Hz), 2.69 (t, 4H, J=4.5 Hz). ¹²C NMR (125 MHz, CDCl₃): δ 155.1, 153.2, 151.0, 149.0, 147.0, 144.7, 140.2, 128.5, 124.5, 120.4, 117.8, 117.0, 55.4, 53.1, 46.9. LC-MS, calculated for C18H18F4N6: 394.15; observed: m/z 395.10 [M+H]+.

### Example 19—Preparation of Compound 6

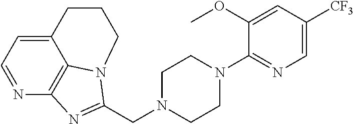 [SMILES: COc1cc(C(F)(F)F)cnc1N1CCN(Cc2nc3nccc4c3n2CCC4)CC1]

In a similar procedure of the reductive amination reaction as described afore, compound 6 was prepared by using the intermediates B and H. ¹H NMR (500 MHz, CDCl₃): δ 8.40 (d, 1H, J=5.0 Hz), 8.07 (s, 1H), 7.09 (s, 1H), 6.92 (d, 1H, J=5.0 Hz), 4.35 (t, 2H, J=6.0 Hz), 3.91 (s, 2H), 3.05 (s, 3H), 3.52 (s, 4H), 2.96 (t, 2H, J=6.0 Hz), 2.67 (t, 4H, J=5.0 Hz), 2.26 (m, 2H). ¹²C NMR (125 MHz, CDCl₃): δ 153.8, 151.6, 145.8, 145.0, 136.1, 130.9, 126.5, 125.2, 123.0, 118.8, 115.4, 113.6, 55.6, 55.5, 53.2, 47.7, 42.9, 23.3, 22.8. LC-MS, calculated for C21H23F3N401: 432.19; observed: m/z 433.00 [M+H]⁺.

### Example 20—Preparation of Compound 7

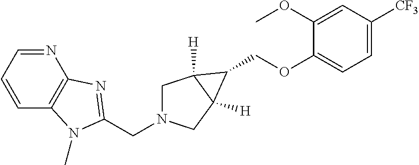 [SMILES: COc1cc(C(F)(F)F)ccc1OC[C@@H]1[C@H]2CN(Cc3nc4ncccc4n3C)C[C@@H]12]

In a similar procedure of the reductive amination reaction as described afore, compound 7 was prepared by using the intermediates A and I. ¹H NMR (500 MHz, CDCl₃): δ 8.47 (d, 1H, J=4.5 Hz), 7.59 (d, 1H, J=7.5 Hz), 7.29 (m, 2H), 7.02 (s, 1H), 6.83 (d, 1H, J=8.5 Hz), 3.93 (s, 2H), 3.85 (s, 3H), 3.83 (d, 2H, J=7.5 Hz), 3.78 (s, 3H), 2.96 (d, 2H, J=9.0 Hz), 2.57 (d, 2H, J=8.0 Hz), 1.67 (m, 1H), 1.50 (s, 2H). ¹²C NMR (125 MHz, CDCl₃): δ 155.0, 154.4, 151.0, 149.3, 144.5, 128.4, 123.1, 122.8, 118.2, 117.6, 116.9, 112.4, 108.4, 71.3, 56.0, 54.5, 51.5, 30.1, 21.5, 18.6. LCMS, calculated for C22H23F3N402: 432.18; observed: m/z 433.15 [M+H]+.

### Example 21—Preparation of Compound 8

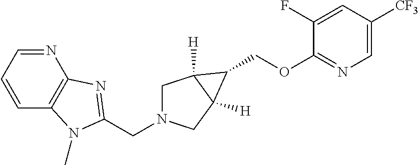 [SMILES: Cn1c(CN2C[C@@H]3[C@@H](COc4ncc(C(F)(F)F)cc4F)[C@@H]3C2)nc2ncccc21]

In a similar procedure of the reductive amination reaction as described afore, compound 8 was prepared by using the intermediates A and J. ¹H NMR (500 MHz, CDCl₃): δ 8.51 (d, 1H, J=4.0 Hz), 8.16 (s, 1H), 7.65 (d, 1H, J=7.5 Hz), 7.49 (d, 1H, J=10.0 Hz), 7.19 (dd, 1H, J=8.0, 5.0 Hz), 4.26 (d, 2H, J=7.0 Hz), 3.99 (s, 2H), 3.83 (s, 3H), 2.98 (d, 2H, J=8.5 Hz), 2.61 (d, 2H, J=8.5 Hz), 1.64-1.67 (m, 1H), 1.51 (s, 2H). ¹²C NMR (125 MHz, CDCl₃): δ 155.5, 154.6, 147.7, 145.6, 144.4, 138.9, 128.4, 124.1, 121.9, 120.2, 117.8, 117.2, 69.4, 54.4, 51.3, 30.2, 21.6, 18.5. LC-MS, calculated for C20H19F4N50: 421.15; observed: m/z 422.10 [M+H]+.

### Example 22—Preparation of Compound 9

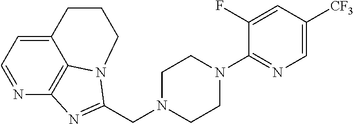 [SMILES: Fc1cc(C(F)(F)F)cnc1N1CCN(Cc2nc3nccc4c3n2CCC4)CC1]

In a similar procedure of the reductive amination reaction as described afore, compound 9 was prepared by using the intermediates B and J. ¹H NMR (500 MHz, CDCl₃): δ 8.40 (d, 1H, J=5.0 Hz), 8.19 (s, 1H), 7.35 (d, 1H, J=13.0 Hz), 6.93 (d, 1H, J=5.0 Hz), 4.34 (t, 2H, J=5.5 Hz), 3.90 (s, 2H), 3.64 (t, 4H, J=4.5 Hz), 2.97 (t, 2H, J=6.0 Hz), 2.66 (t, 4H, J=4.5 Hz), 2.27 (m, 2H). ²C NMR (125 MHz, CDCl₃): δ 153.8, 151.1, 149.0, 147.0, 145.1, 140.1, 130.9, 126.5, 124.5, 122.3, 120.3, 115.4, 55.3, 53.1, 46.9, 42.8, 23.3, 22.8. MS (ES+): m/z 421.15 (M⁺¹). LC-MS, calculated for C20H20F4N5: 420.17; observed: m/z 421.15 [M+H]+.

### Example 23—Preparation of Compound 10

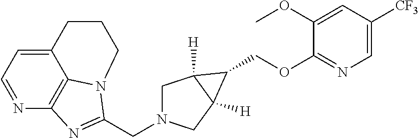 [SMILES: COc1cc(C(F)(F)F)cnc1OC[C@@H]1[C@H]2CN(Cc3nc4nccc5c4n3CCC5)C[C@@H]12]

In a similar procedure of the reductive amination reaction as described afore, compound 10 was prepared by using the intermediates B and K. ¹H NMR (500 MHz, CD₃OD): δ 8.41 (d, 1H, J=5.0 Hz), 7.16 (d, 1H, J=8.5 Hz), 7.06 (s, 1H), 6.93 (d, 1H, J=5.0 Hz), 6.87 (d, 1H, J=8.5 Hz), 4.21 (t, 2H, J=5.5 Hz), 3.96 (s, 2H), 3.89 (s, 3H), 3.87 (d, 2H, J=7.5 Hz), 3.00 (d, 2H, J=9.0 Hz), 2.96 (t, 2H, J=6.0 Hz), 2.58 (d, 2H, J=8.5 Hz), 2.21-2.25 (m, 2H), 1.68-1.71 (m, 1H), 1.49 (s, 2H). ²C NMR (125 MHz, CDCl₃): δ 153.9, 152.5, 150.9, 149.4, 145.0, 130.7, 126.4, 118.2, 115.3, 112.5, 112.5, 108.4, 71.4, 56.1, 54.6, 51.5, 42.7, 23.3, 22.9, 21.6, 18.6. LC-MS, calculated for C23H24F3N502: 459.19; observed: m/z 460.15 [M+H]+.

### Example 24—Preparation of Compound 11

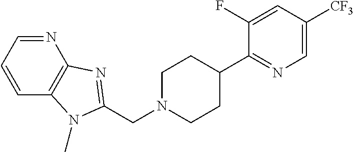 [SMILES: Cn1c(CN2CCC(c3ncc(C(F)(F)F)cc3F)CC2)nc2ncccc21]

In a similar procedure of the reductive amination reaction as described afore, compound 11 was prepared by using the intermediates A and G. ¹H NMR (500 MHz, CDCl₃): δ 8.61 (s, 1H), 8.51 (d, 1H, J=5.0 Hz), 7.64 (d, 1H, J=8.0 Hz), 7.53 (d, 1H, J=9.5 Hz), 7.17 (dd, 1H, J=8.0, 5.0 Hz), 3.92 (s, 3H), 3.90 (s, 2H), 3.12 (t, 1H, J=11.5 Hz), 3.01 (d, 2H, J=11.5 Hz), 2.34 (t, 2H, J=11.5 Hz), 1.96 (qd, 2H, J=11.5, 3.0 Hz), 1.79 (m, 2H). ¹²C NMR (125 MHz, CDCl₃): δ 157.4, 157.1, 156.9, 155.3, 155.2, 154.0, 144.6, 141.6 128.7, 119.9, 117.6, 116.9, 56.0, 53.8, 37.2, 30.3, 30.2. LC-MS, calculated for C19H19F4N5: 393.16; observed: m/z 394.10 [M+H]+.

### Example 25—Preparation of Compound 12

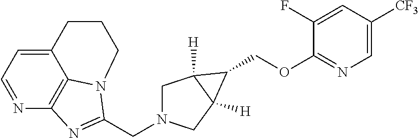 [SMILES: Fc1cc(C(F)(F)F)cnc1OC[C@@H]1[C@H]2CN(Cc3nc4nccc5c4n3CCC5)C[C@@H]12]

In a similar procedure of the reductive amination reaction as described afore, compound 12 was prepared by using the intermediates B and J. ¹H NMR (500 MHz, CDCl₃): δ 8.40 (d, 1H, J=5.0 Hz), 8.16 (s, 1H), 7.50 (d, 1H, J=9.5 Hz), 6.97 (d, 1H, J=5.0 Hz), 4.27 (d, 2H, J=7.0 Hz), 4.24 (t, 2H, J=5.5 Hz), 4.02 (s, 2H), 3.02 (d, 2H, J=9.5 Hz), 2.97 (t, 2H, J=5.5 Hz), 2.65 (d, 2H, J=9.0 Hz), 2.24-2.28 (m, 2H), 1.64-1.67 (m, 1H), 1.53 (s, 2H). ¹²C NMR (125 MHz, CDCl₃): δ 164.4, 155.4, 152.5, 147.7, 145.7, 144.6, 139.0, 131.9, 126.5, 124.1, 120.1, 115.7, 69.3, 54.5, 50.8, 42.8, 23.2, 22.8, 21.6, 18.7. LC-MS, calculated for C22H21F4N50: 447.17; observed: m/z 448.20 [M+H]+.

### Example 26—Preparation of Compound 13

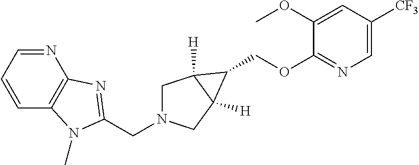 [SMILES: COc1cc(C(F)(F)F)cnc1OC[C@@H]1[C@H]2CN(Cc3nc4ncccc4n3C)C[C@@H]12]

In a similar procedure of the reductive amination reaction as described afore, compound 13 was prepared by using the intermediates A and K. ¹H NMR (500 MHz, CDCl₃): δ 8.41 (d, 1H, J=5.0 Hz), 7.89 (s, 1H), 7.54 (d, 1H, J=7.5 Hz), 7.09 (dd, 1H, J=8.0, 4.5 Hz), 7.07 (s, 1H), 4.18 (d, 1H, J=7.5 Hz), 3.88 (s, 2H), 3.81 (s, 3H), 3.73 (s, 3H), 2.90 (d, 1H, J=9.0 Hz), 2.51 (q, 1H, J=9.0 Hz), 1.63 (m, 1H), 1.44 (s, 2H). ¹²C NMR (125 MHz, CDCl₃): δ 156.3, 154.9, 154.5, 144.3, 143.9, 134.4, 128.4, 124.9, 119.7, 117.6, 116.9, 113.4, 69.0, 55.9, 54.4, 51.4, 30.0, 23.8, 21.6, 18.5. LC-MS, calculated for C21H22F3N502: 433.17; observed: m/z 434.15 [M+H]+.

### Example 27—Preparation of Compound 14

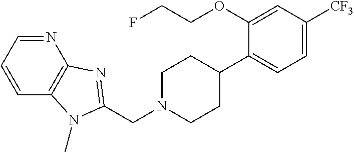 [SMILES: Cn1c(CN2CCC(c3ccc(C(F)(F)F)cc3OCCF)CC2)nc2ncccc21]

In a similar procedure of the reductive amination reaction as described afore, compound 14 was prepared by using the intermediates A and L. (Yellow solid, 24.0 mg, 55%). LCMS: m/z 437.2 [M+H]+, ¹H NMR (300 MHz, methanol-d₄) δ ppm 1.76-1.82 (m, 4H), 2.31-2.39 (m, 2H), 3.02-3.06 (m, 3H), 3.93 (s, 2H), 4.01 (s, 3H), 4.24-4.34 (m, 2H), 4.68-4.78 (m, 2H), 7.17-7.23 (m, 2H), 7.33-7.38 (m, 2H), 8.00-8.03 (m, 1H), 8.39-8.41 (m, 1H). ¹³C NMR (75 MHz, methanol-d4) δ ppm 156.2, 154.9, 153.7, 143.6, 138.8, 128.9, 126.9, 124.2 (q, J=271.5 Hz), 118.7, 118.0, 117.5, 108.3, 81.6 (q, J=169.5 Hz), 68.1, 67.8, 54.5, 54.1, 35.4, 31.3, 29.5. LC-MS, calculated for C21H22F3N50: 436.19; observed: m/z 437.2 [M+H]+.

### Example 28—Preparation of Compound 15

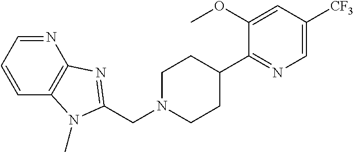 [SMILES: COc1cc(C(F)(F)F)cnc1C1CCN(Cc2nc3ncccc3n2C)CC1]

In a similar procedure of the reductive amination reaction as described afore, compound 15 was prepared by using the intermediates A and M. (Pale yellow solid, Yield 35%). ¹H NMR (300 MHz, methanol-d4) δ ppm 1.76-1.82 (m, 2H), 1.86-2.0 (m, 2H), 2.29-2.37 (m, 2H), 3.01-3.05 (m, 2H), 3.21-3.26 (m, 1H), 3.91-3.95 (m, 5H), 4.0 (s, 3H), 7.31-7.35 (m, 1H), 7.52 (s, 1H), 7.99-8.02 (m, 1H), 8.32 (s, 1H), 8.39-8.41 (m, 1H). ¹³C NMR (75 MHz, methanol-d4) δ ppm 158.5, 154.9, 153.6, 153.2, 143.6, 136.1, 128.9, 124.8, 123.7 (q, J=271.8 Hz), 118.7, 118.0, 113.5, 55.0, 53.8, 53.4, 37.0, 29.8, 29.6. LC-MS, calculated for C20H22F3N50: 405.18; observed: m/z 406.1 [M+H]+.

### Example 29—Preparation of Compound 16

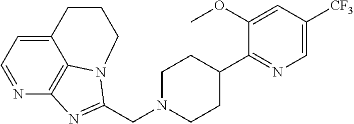 [SMILES: COc1cc(C(F)(F)F)cnc1C1CCN(Cc2nc3nccc4c3n2CCC4)CC1]

Compound 16. In a similar procedure of the reductive amination reaction as described afore, compound 16 was prepared by using the intermediates B and M. (Pale yellow solid, Yield 31%) 1H NMR (300 MHz, DMSO-d6) δ ppm 1.67-1.82 (m, 5H), 2.14-2.25 (m, 4H), 2.92-2.95 (m, 4H), 3.85 (s, 2H), 3.90 (s, 3H), 4.31-4.35 (m, 2H), 7.0 (d, J=4.9 Hz, 1H), 7.63 (s, 1H), 8.24 (d, J=4.9 Hz, 1H), 8.45 (s, 1H). ¹³C NMR (75 MHz, DMSO-d6) δ ppm 158.7, 154.0, 153.0, 152.6, 144.3, 136.9, 131.9, 126.6, 124.2 (q, J=272.4 Hz), 124.0, 124.1, 115.5, 114.7, 56.6, 55.3, 53.8, 42.9, 37.2, 30.5, 23.2, 22.8. LC-MS, calculated for C22H24F3N50: 431.19; observed: m/z 432.1 [M+H]+.

### Example 30 —mGluR2 PAM Activity of Exemplified Compounds Determined by GloSensor cAMP Assay

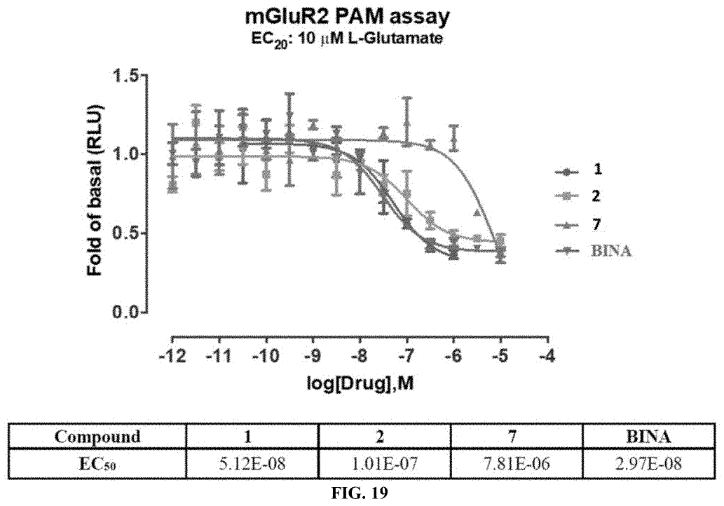

HEK-293 cells were maintained with complete Dulbecco's modified Eagle's medium (DMEM), which is composed of 10% fetal bovine serum (FBS), 2 μM L-glutamine, 100 units/mL penicillin G, 100 μg/mL streptomycin at 37° C. in the presence of 5% CO₂. HEK-293 stable cell lines with tetracycline inducible expression of mGluR2 were maintained with complete DMEM with 100 μg/mL Hygromycin B, 15 μg/mL Blasticidin at 37° C. in the presence of 5% CO₂. The mGluR2 (a Gi/o coupled receptor) PAM activity was tested by using Promega's split luciferase based GloSensor cAMP biosensor technology. On cells stably expressing mGluR2, GloSensor cAMP DNA construct was transfected overnight. Cells were seeded into PLL coated 384-well white clear bottom cell culture plates with complete BME buffer with 1 μg/mL tetracycline at a density of 20,000 cells for another 24 h. Cell medium was removed and was incubated with 20 PL of 4 mM luciferin (Gold Biotechnology) for 1 h at 37° C. To measure PAM activity of mGluR2, 10 μL of 3× test compound solution and an EC₂₀-equivalent concentration of glutamate (10 μM) were added for 15 min before addition of 10 μL of isoproterenol at a final concentration of 200 nM, followed by counting of the plate for chemiluminescence for 15 min. In the experiment, isoproterenol was used to activate endogenous P2 adrenergic receptors expressed in HEK293 T cells to activate the endogenous Gs protein. Luminescence was counted in a Trilux luminescence counter. Data were analyzed with Prizm 5.0 (GraphPad software). The results of the assay for the FIG. 19.

### Example 31—Synthesis of the Tritium-Labeled Compound [³H]1

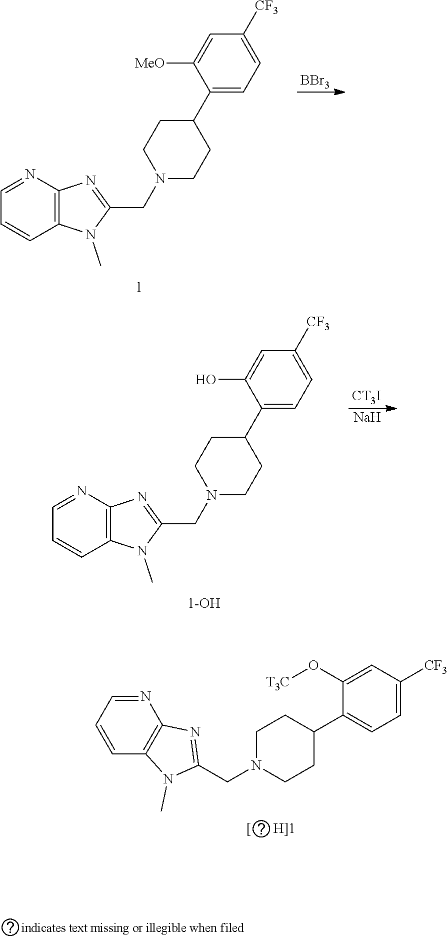

To a solution of compound 1 (70 mg, 0.173 mM) in 3 mL of dichloromethane at 0° C. was added boron tribromide solution (1 mL, 1M in DCM, 1 mM) dropwise. The mixture was slowly warmed to room temperature and stirred for 2 h, and the reaction was monitored by TLC. After 5 ml of saturated sodium bicarbonate was added, the crude product was extracted with dichloromethane and purified by silica-gel column chromatography to give compound 1-OH (60 mg, 0.153 mM, 88% yield). ¹H NMR (500 MHz, CD₃OD): δ 8.42 (d, 1H, J=5.0 Hz), 8.03 (d, 1H, J=8.5 Hz), 7.34-7.37 (m, 1H), 7.28 (d, 1H, J=7.5 Hz), 7.05 (d, 1H, J=7.5 Hz), 7.00 (s, 1H), 4.01 (m, 3H), 3.94, (m, 2H), 2.99-3.06 (m, 3H), 2.34-2.38 (m, 2H), 1.73-1.86 (m, 4H). ¹²C NMR (125 MHz, CD₃OD): δ 155.4, 155.0, 153.7, 143.6, 136.6, 128.7, 126.8, 125.4, 123.3, 118.7, 118.0, 115.3, 115.3, 111.0, 54.6, 54.2, 35.1, 31.3, 29.5. LC-MS, calculated for C20H21F3N40: 390.17; observed: 391.10 [M+H]⁺.

To a mixture solution of compound 1-OH (2 mg, 5 μM) and NaH (10 mg, 60% dispersion in mineral oil) in dimethylformamide (0.3 mL) was added [³H]methyl iodide (25 mCi, 1 Ci/mL in dimethylformamide, 80 Ci/mM specific activity). The reaction mixture was heated at 90° C. for 10 min. After cooled down to room temperature the reaction solution was diluted with 4 ml of HPLC solvents (acetonitrile/water 60/40), then injected into HPLC equipped with Gemini-NX C18 semipreparative column (250 mm×10 mm, 5 μm, Phenomenex Inc.), flow scintillation detector, and internal UV detector. Compounds was eluted from column with a solution of 60% acetonitrile and 0.1M ammonium formate at a flow rate of 5 mL/min. The fractions containing compound [³H]1 were collected between 10-11 min. The radioactive product was concentrated through a C₁₈ Sep-Park Plus cartridge and eluted with ethanol to give 0.5 mCi of [³H]1 (4% yield).

### Example 32—Radiosynthesis of the C-11 Labeled Compound [¹¹C]1

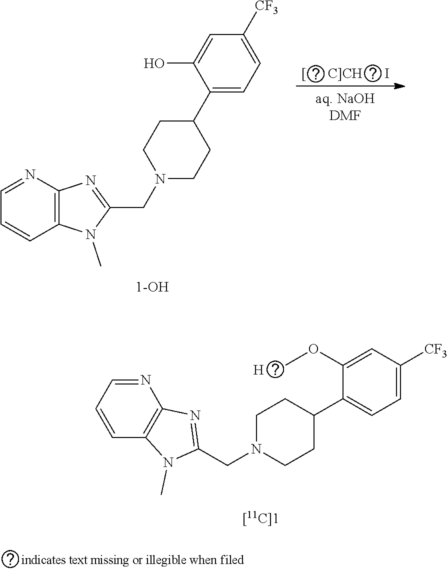

¹¹CO₂ was obtained via the ¹⁴N(p,α)¹¹C reaction on nitrogen with 2.5% oxygen, with 16 MeV protons (GE PET Trace), and trapped on molecular sieves in a TRACERab FX-Mel synthesizer (GE). ¹¹CH₄ was obtained by the reduction of ¹¹CO₂ in the presence of hydrogen at 350° C. and passed through an oven containing I₂ to produce ¹¹CH₃I via a radical reaction. ¹¹CH₃I was trapped in a TRACERab FX-M synthesizer reactor (General Electric) preloaded with a solution of excess 1-OH (0.7±0.2 mg) and an aqueous 5N NaOH (3-5 μL) in dry dimethylformamide (300 μL) at room temperature for 3 min and then heated at 80° C. for 3 min. The reaction mixture was diluted with 1.5 mL of water and purified using a HPLC system equipped with a semi-preparative column (Waters XBridge, C₁₈, 250×10 mm, 5μ), a UV detector monitored at 254 nm, and a radioactivity detector. The product was purified by HPLC eluted with acetonitrile/water/TFA (30/70/0.7) at a flow rate of 5 mL/min. The fractions corresponding to [¹¹C]1 (t_{R}=12 min) were collected into a large dilution vessel, which was pre-loaded with 2 mL of 8.4% sodium bicarbonate for injection, USP (Hospira) and 23 mL of sterile water for injection, USP. The product was loaded onto a C₁₈ light cartridge, (Waters; preactivated with 5 mL of EtOH followed by 10 mL of SWFI). The C₁₈ light cartridge was washed with 10 mL of SWFI to remove traces of salts, residual MeCN, and TFA. The C₁₈ light cartridge was then eluted with 1 mL dehydrated ethyl alcohol (USP) and followed by 10 mL 0.9% sodium chloride solution (USP) into a product collection vessel. The formulated solution was filtered through a vented Millipore-GV 0.22 μl sterilizing filter (EMO Millipore) into a 10 mL vented sterile vial. Radiochemical purity and chemical quality were checked by analytical HPLC equipped with a analytical column (Waters, XBridge, C₁₈, 3.5μ, 4.6×150 mm), a UV detector monitored at 254 nm, and a radioactivity detector, which was eluted with a solution (acetonitrile/0.1% TFA water=30/70) at a flow rate of 1 mL/min. [¹¹C]1 was eluted around 6 min (chemical purity >90%, radiochemical purities >95%, n=2). The radiosynthesis took 50 min from the end of bombardment (EOB) to the end of synthesis (EOS), no radiolysis was observed up to 90 min. The carbon-11 labeled compound [¹¹C]1 was obtained in 20±2% RCYs (decay corrected) from [¹¹C]CO₂. Its specific activity at the end of synthesis was 98±30 GBq/μmol at the end of synthesis (EOS). The [¹¹C]1 was then formulated into 10% ethanolic saline solution (pH=5-6) before injection.

### Example 33—Radiosynthesis of the C-11 Labeled Compounds [¹¹C]2

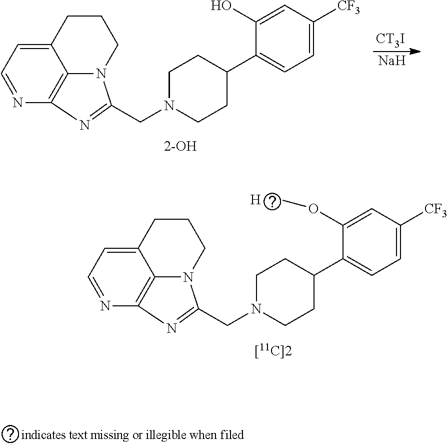

¹¹CO₂ was obtained via the ¹⁴N(p,α)¹¹C reaction on nitrogen with 2.5% oxygen, with 16 MeV protons (GE PET Trace), and trapped on molecular sieves in a TRACERab FX-Mel synthesizer (GE). ¹¹CH₄ was obtained by the reduction of ¹¹CO₂ in the presence of hydrogen at 350° C. and passed through an oven containing I₂ to produce ¹¹CH₃I via a radical reaction. ¹¹CH₃I was trapped in a TRACERab FX-M synthesizer reactor (General Electric) preloaded with a solution of excess 2-OH (0.3 mg) and an aqueous 1N NaOH (3 μL) in dry dimethylformamide (250 μL) at room temperature and then heated at 80° C. for 2 min. The reaction mixture was diluted with 1.0 mL of water and purified using a HPLC system equipped with a semi-preparative column (Waters XBridge, C₁₈, 250×10 mm, 5μ), a UV detector monitored at 254 nm, and a radioactivity detector. The product was purified by HPLC eluted with acetonitrile/water/TFA (30/70/0.7) at a flow rate of 5 mL/min. The fractions corresponding to [¹¹C]2 (t_{R}=11 min) were collected into a large dilution vessel, which was pre-loaded with 2 mL of 8.4% sodium bicarbonate for injection, USP (Hospira) and 23 mL of sterile water for injection, USP. The product was loaded onto a C₁₈ light cartridge, (Waters; pre-activated with 5 mL of EtOH followed by 10 mL of SWFI). The C₁₈ light cartridge was washed with 10 mL of SWFI to remove traces of salts, residual MeCN, and TFA. The C₁₈ light cartridge was then eluted with 1 mL dehydrated ethyl alcohol (USP) and followed by 10 mL 0.9% sodium chloride solution (USP) into a product collection vessel. The formulated solution was filtered through a vented Millipore-GV 0.22μ sterilizing filter (EMO Millipore) into a 10 mL vented sterile vial. Radiochemical purity and chemical quality were checked by analytical HPLC equipped with an analytical column (Waters, XBridge, C₁₈, 3.5μ, 4.6×150 mm), a UV detector monitored at 254 nm, and a radioactivity detector, which was eluted with a solution (acetonitrile/0.1% TFA water=30/70) at a flow rate of 1 mL/min. [¹¹C]2 was eluted around 6 min (chemical purity >98%, radiochemical purities >98%, n=1). The radiosynthesis took 50 min from the end of bombardment (EOB) to the end of synthesis (EOS). Its specific activity at the end of synthesis was 91 GBq/μM at the end of synthesis (EOS).

### Example 34—Radiosynthesis of the F-18 Labeled Compound [¹⁸F]3

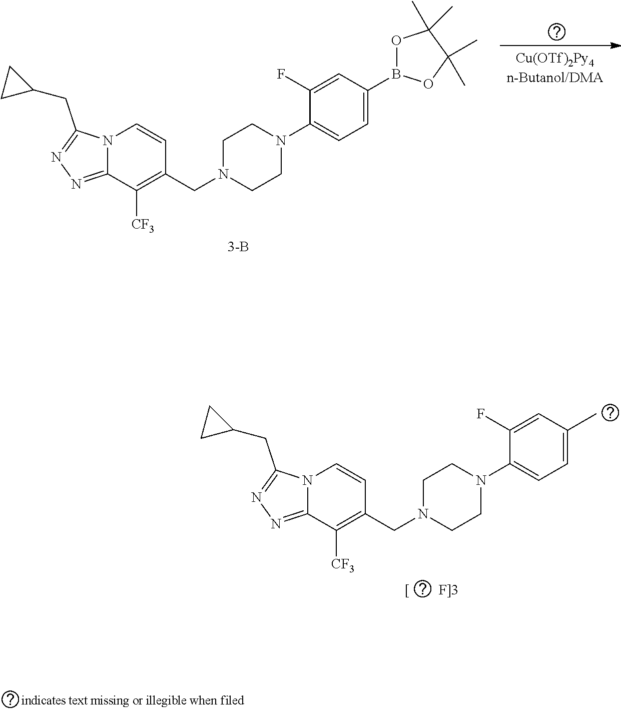

Aqueous [¹⁸F]fluoride was loaded onto an anion-exchange resin (Chromafix PS-HCO₃ cartridge). The resin was washed with acetone three times and flushed with air to remove acetone. [¹⁸F]fluoride (5 mCi) was eluted from a Chromafix PS-HCO3 cartridge with a solution of tetraethylammonium bicarbonate (2.7 mg, 0.7 FM) in n-Butanol (400 μL). The solution was added tetrakis(pyridine)copper(II) triflate (18 mg, 26.5 μM), the boronic ester precursor 3-8 (24.38 mg, 60 μM) in anhydrous N,N-Dimethylacetamide (800 μL). The mixture was heated at 110° C. for 20 min. The reaction mixture was cooled to room temperature, diluted with water (2 mL) and shaken vigorously for 30 s. Thereafter, the RCY was determined by radio-HPLC (5%, n=3).

### Example 35—in Silico Evaluation of Compounds 1, 2, and 7

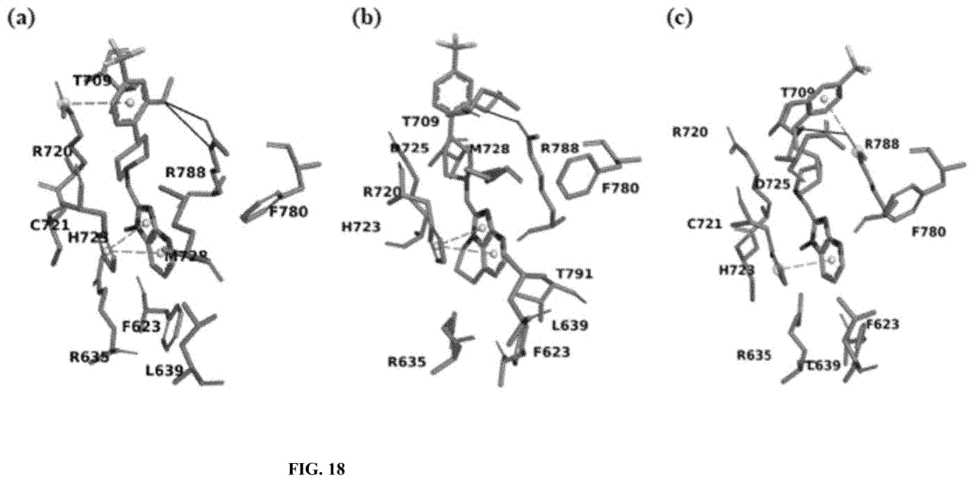

To provide structural insights on ligand-protein binding, compounds 1, 2, and 7 were docked into a mGluR2 homology model, which was built in YASARA [see ref. 40] and validated by a series of structural analysis tools of ModFOLD [see ref. 41], ERRAT and VERIFY 3D (See supporting information) [see refs. 42-44]. The key binding residues were predicted by Partial Order Optimum Likelihood (POOL) [see ref. 45], DEPTH [see ref. 46] and MetaPocket [see ref. 47]. The docking experiments were performed at the 7-TM region with AutoDock [see ref. 48] embedded in YASARA. As shown in FIG. 18, compounds 1, 2, and 7 localize similarly at the entrance of the 7-TM region with their heterocyclic cores projecting to the bottom hydrophobic pocket and the distal substituted arenes interacting with residues at the extracellular loop 2 (EL2). Compound 1 has the best docking score of 8.7 kcal/M compared to the values of 8.6 kcal/M and 7.3 kcal/M for compounds 1 and 7, respectively. Compound 1 shows a hydrogen bonding interaction with Arg788, a n-cation interaction with Arg720, and a π-π stacking interaction with His723 (FIG. 18). His723 has been previously reported as a key hydrophobic residue that interacts with several mGluR2 PAMs [see refs. 49, 50]. Compound 2 has similar key binding interactions as that of compound 1, whereas, compound 7 exhibited fewer contacts in the binding pocket than compounds 1 and 2, consistent with the decreased docking score. Overall, the in silico simulations suggest compounds 1, 2, and 7 as potent mGluR2 binding ligands. A further validation of this observation with in vitro experiments is illustrated in these examples.

### Example 36—In Vitro Characterization of Compounds 1, 2, and 7

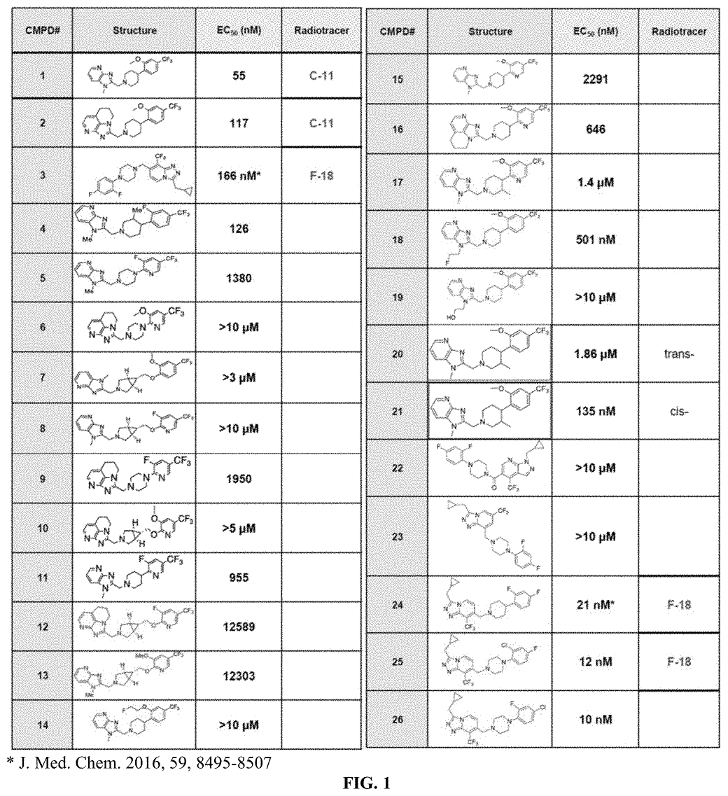

The in vitro characterization of compounds 1, 2, and 7 were studied by a series of assays to test their mGluR2 PAM activity, selectivity to other mGluRs, lipophilicity, plasma protein binding, metabolic and solution stabilities and BBB penetration properties. The mGluR2 PAM activity of compounds 1, 2, and 7 was determined using Promega's split luciferase based GloSensor cAMP biosensor assay [see refs. 51, 52]. Previously reported EC₅₀ values for compounds 1 and 2 (EC₅₀=13 nM and 5 nM, respectively) [see refs. 36, 37] were determined by forced-coupling of mGluR2 to Gα15 or Gα16 followed by fluorescence detection of calcium flux upon activation. However, this assay is sub-optimal as it does not signal through the biorelevant cAMP pathway. Here, with this live cell assay, the mGluR2 PAM activity of compounds 1, 2, and 7 was evaluated in the presence of EC₂0 amount of L-glutamate by measuring changes in intracellular cAMP concentration, the relevant second messenger mechanism. An mGluR2 PAM, [3′-(((2-cyclopentyl-6,7-dimethyl-1-oxo-2,3-dihydro-1H-inden-5-yl)oxy)methyl)-[1,1′-biphenyl]-4-carboxylic acid](BINA):

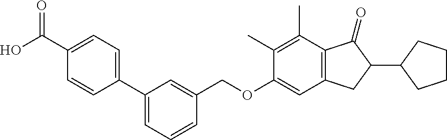 [SMILES: Cc1c(OCc2cccc(-c3ccc(C(=O)O)cc3)c2)cc2c(c1C)C(=O)C(C1CCCC1)C2]

[see ref. 53], was used as the reference compound for the assay. As FIG. 1 shows, the EC₅₀ values of 1, 2 and 7 are 55 nM, 117 nM and 778 nM, respectively, suggesting that 1 is a very potent mGluR2 PAM. The selectivity of 1, 2, and 7 was also analyzed among the various mGluR subtypes, in which the Gq coupled receptors (mGluR1 and mGluR5) were tested using Ca² mobilization assay and the Gi/o coupled receptors (mGluR2, mGluR3, mGluR4, mGluR6 and mGluR8) using cAMP assay. Results demonstrate that 1 has good selectivity against other mGlu receptors (>100-fold, FIG. 17).

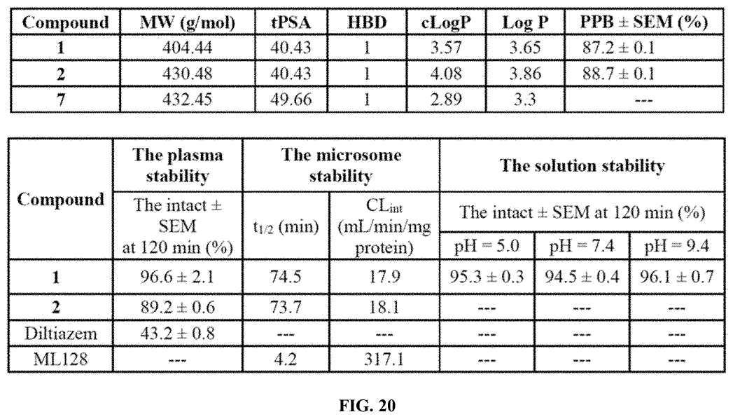

The pharmacological properties of compounds 1, 2, and 7 were determined via ChemBiodraw (version 16.0) based on the molecular weight (MW), topological polar surface area (tPSA), and c Log P (FIG. 20). The experimental lipophilicity was measured by using liquid-liquid partition between n-octanol and water (“shake-flask method”) [see ref. 54]. The Log P values obtained for compounds 1, 2, and 7 were 3.65, 3.86 and 3.30, respectively, indicating their satisfactory CNS penetrating potentials (FIG. 5) [see ref. 55]. The plasma protein binding comprises compounds' binding to albumin, α1-acid glycoprotein and lipoproteins once delivered to the bloodstream. This property was evaluated for compounds 1 and 2 by equilibrium dialysis [see ref. 56], where two chambers were separated by a dialysis membrane (MWCO 8 kD). The plasma protein bindings of 1 and 2 are 87.2% and 88.7%, respectively (see FIGS. 20 and 6). Therefore, the high plasma free fraction of compounds 1 and 2 (>10%) would allow enough free drug concentration in blood stream to reach the brain targets.

The in vitro plasma and liver microsomal stability of 1 and 2 were studied by incubating the test compounds in rat serum and rat liver microsomes as well as NADPH cofactor, respectively, using previously published methods [see refs. 57, 58]. Diltiazem and ML128 (a mGluR4 PAM) [see refs. 59, 60] were used as co-assay QC controls for plasma and microsomal stability assays, respectively, to ensure that the assays were operating properly, and that the activity of the plasma and microsomes were consistent with established criteria. Compounds 1 and 2 are much more stable than diltiazem in rat plasma (FIGS. 10 and 20). The results also show that 1 and 2 exhibit reasonable microsomal stability and are much more stable than ML128, in which the suitable hepatic clearance of 1 and 2 is predicted (FIGS. 8, 9, and 20). The solution stability of 1 was evaluated with buffer solutions at pH 5.0, 7.4 and 9.4, respectively (FIGS. 10 and 20) [see refs. 61]. The results indicate that 1 is relatively stable in pH ranging from 5.0 to 9.4.

### Example 37—Assessment of BBB Permeability for Compounds 1, 2, and 7

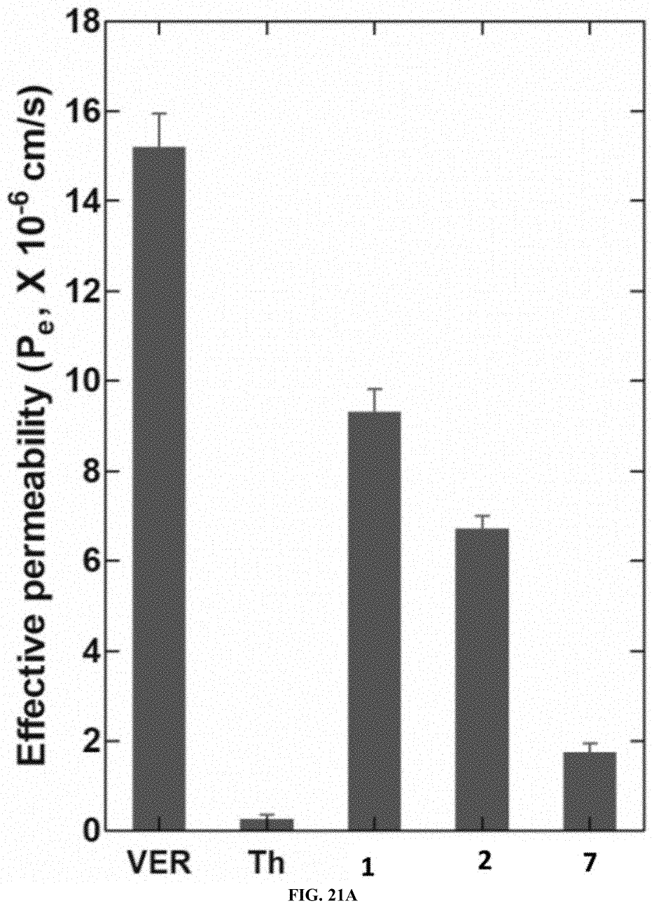

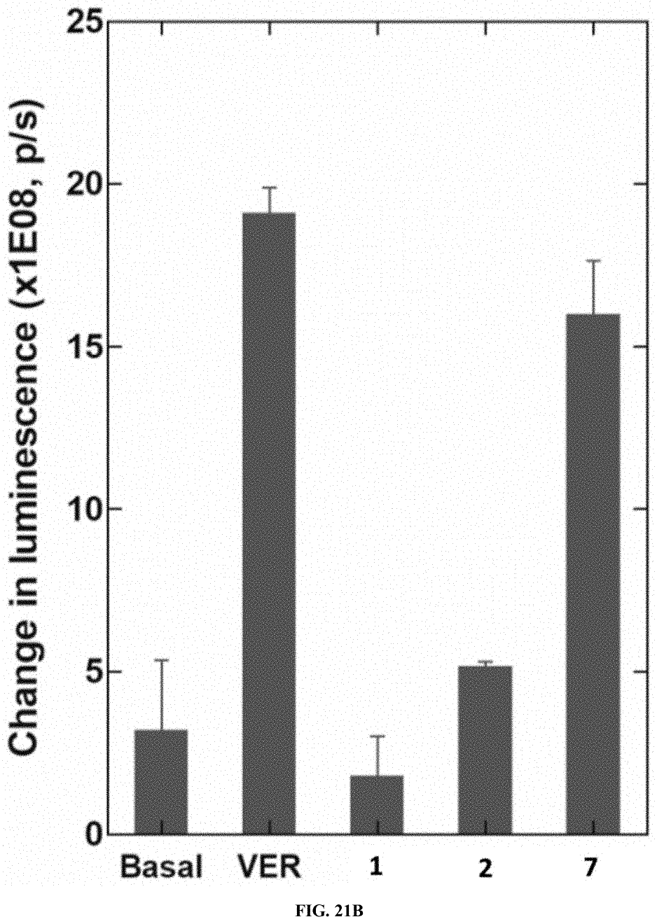

BBB penetration was a major barrier for some previously reported mGluR2 PET tracers that otherwise could have efficacy for imaging the brain target as shown by radiotracers described in references 31 and 32 (also shown in FIG. 6). BBB penetration potential of compounds 1, 2, and 7 was studied with two in vitro assays, namely, parallel artificial membrane permeability assay (PAMPA) and Pgp-Go™ assay. The PAMPA assay was carried out to predict passive BBB permeability [see ref. 62]. Quality control standards were run with each sample set to monitor the consistency of the analysis. Verapamil was used as a high permeability standard (Pₑ=16×10⁻⁶ cm/s) and theophylline was used as a low permeability standard (Pₑ=0.12×10⁻⁶ cm/s). As FIG. 21A shows, compound 1 has the best membrane permeability with an average effective permeability (Pₑ) value of 9.3×10⁻⁶ cm/s.

We further evaluated compounds 1, 2, and 7 using Pgp-Go™ assay to investigate whether the brain penetration will be affected by P-glycoprotein (P-gp) efflux transporter. The Pgp-Go™ assay [see ref. 63] was carried out on recombinant human P-gp in a cell membrane fraction. The effect of compounds 1, 2, and 7 on P-gp ATPase activity was examined by comparing the untreated samples and the samples treated with 1, 2, and 7 to sodium orthovanadate (Na₃VO₄)-treated control. The difference in luminescent signal between Na₃VO₄-treated samples and samples treated with the test compounds implied P-gp ATPase activity in the presence of the test compound. Verapamil, a P-gp substrate, was used as a positive control in the assay. By comparing basal and verapamil activities to that of 1, 2, and 7, it is clearly indicated that 1 is not a P-gp substrate and 7 is a potential P-gp substrate, while 1 displays a moderate P-gp ATPase activity (FIG. 21B).

The in vitro pharmacological studies reveal that compound 1 has many CNS drug-like properties, including the potent mGluR2 PAM activity and good selectivity against other mGluRs, suitable lipophilicity and PPB, adequate metabolic stability, favorable passive permeability as measured by PAMPA, and no P-gp liability. Based on these results, compound 1 was selected for the radiolabeling and for in vivo evaluation as mGluR2 PET radioligand.

### Example 38—Whole Body Biodistribution of [¹¹C]1

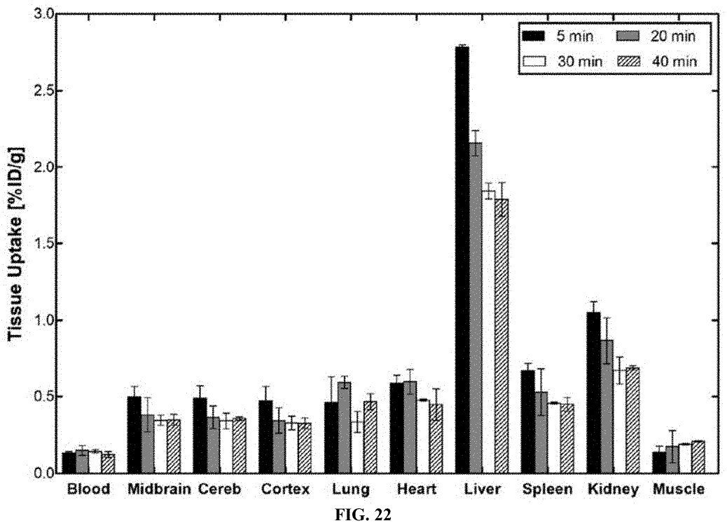

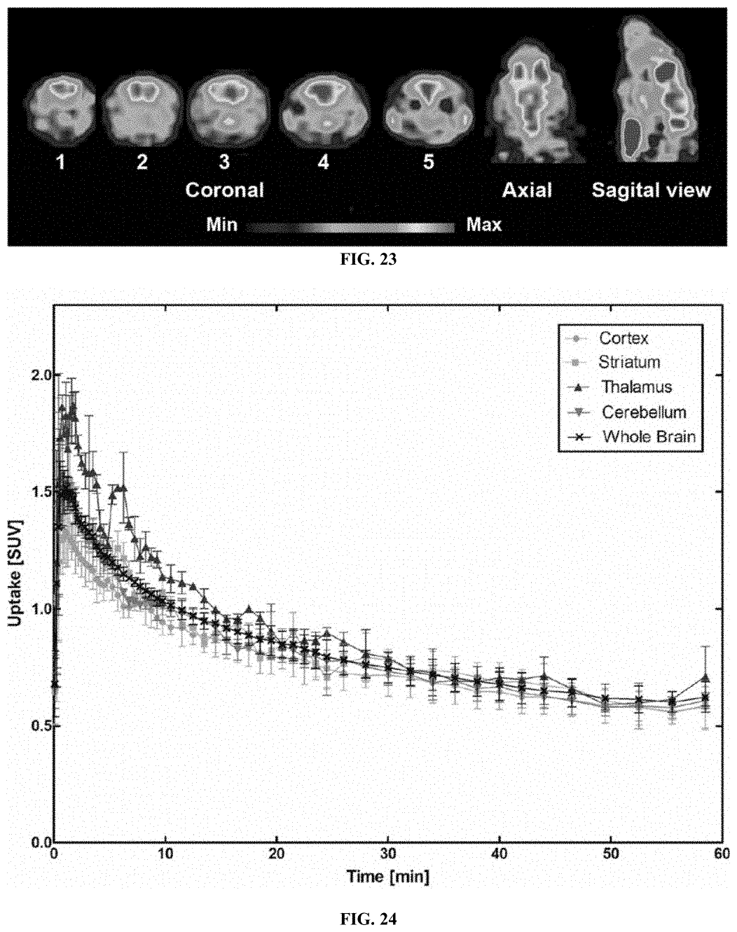

The ex vivo biodistribution was performed in 16 normal male Sprague Dawley rats after intravenous injection of [¹¹C]1 at several time points (5, 20, 30 and 40 min). The uptake value is expressed in the unit of % ID/g. These studies support reversible accumulation of [¹¹C]1 with the highest accumulation 5 min after administration of radioactivity in other investigated tissue areas but the lungs where the maximum accumulation was at 20 min and the muscle where the radioactivity steadily increased up to 40 min (FIG. 22). The highest accumulation was measured in the liver (2.73±0.02% ID/g) followed by kidney (1.05±0.07% ID/g), spleen (0.67±0.05% ID/g), lung (0.59±0.04% ID/g), and heart (0.58±0.05% ID/g). The high radioactivity uptake in liver and kidney suggest that hepatobiliary elimination and renal excretion contribute to the whole body distribution of [¹¹C]1. The average accumulation of [¹¹C]1 in the rat brain at 5 min was (0.49±0.07% ID/g) (FIG. 22). This result indicates a rapid BBB penetration of [¹¹C]1, which was consistent with the in vivo brain imaging studies described in these examples.

### Example 39—PET Imaging of [¹¹C]1

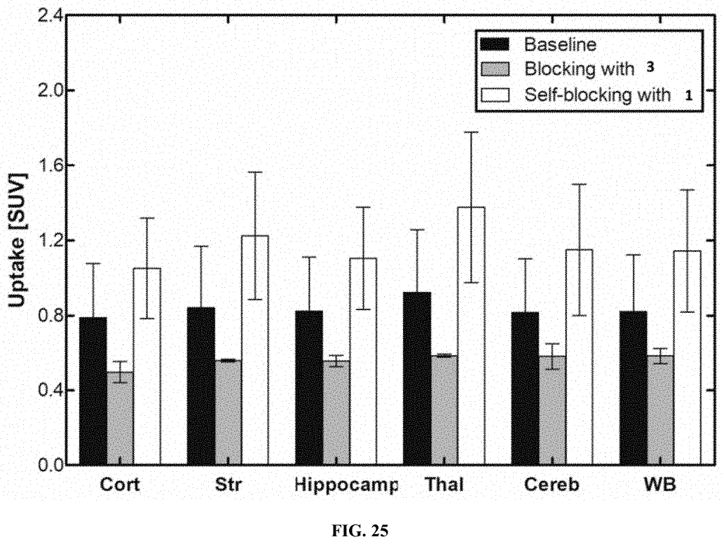

The characterization of [11C]1 was conducted with rat (male Sprague-Dawley) studies. All animal studies were performed by the guidance of the National Institute of Health guide for the care and use of laboratory animals and, approved and supervised under the subcommittee on research animals of the Harvard Medical School and Massachusetts General Hospital. For the imaging studies rats were anaesthetized with isoflurane/nitrous oxide (1.0-1.5% isoflurane, with oxygen flow of 1-1.5 L/min) and the tail vein was catheterized for administration of the imaging ligands ([¹¹C]1) and blocking agents. The rats were adjusted into the scanner for imaging position (Triumph II, Trifoil Imaging Inc, Northridge, Calif.). The vital signs such as heart rate and/or breathing were monitored throughout the imaging. In vivo PET imaging studies were conducted in twelve rats, altogether sixteen studies which included nine for baseline studies with [¹¹C]1 (control), four for self-blocking by compound 1 (4 mg/kg), three for blocking by a mGluR2-selective ligand compound 3 (JNJ-46356479, 4 mg/kg). When the blocking studies were done the imaging procedures started with the baseline study using [¹¹C]1 followed by 2 hrs later the blocking agent and another administration of [¹¹C]1 in the same animal. The blocking agent was administered intravenously 2 min before injection of the second [¹¹C]1 if self-blocking, 10 min before [¹¹C]1 if blocking was with JNJ The dynamic PET data was acquired starting from the injection of radioactivity and continued for 60 min. After the dynamic PET data acquisition CT imaging was done to obtain data for attenuation as well as anatomical information of the brain. The dynamic volumetric PET images were reconstructed after correction for attenuation, uniformity and scatter using a software, “Maximum Likelihood Expectation Maximization, MLEM” provided by the Manufacturer using 30 iterations. The CT data was reconstructed by a modified Feldkamp algorithm using matrix volumes of 512×512×512 and pixel size of 170 μm. Regions of interests (ROI)s including striatum, thalamus, hippocampus, different cortical areas, cerebellum and whole brain were drawn onto coronal slices according to the brain outlines as derived from the rat brain atlas and corresponding time-activity curves (TACs) were created by PMOD 3.2 (PMOD Technologies Ltd, Zurich, Switzerland). Percent changes between the control and blocking studies were calculated in all brain areas. Representative PET images of cumulative volumetric distribution of [¹¹C]1 at time interval of 10-15 min are shown on five coronal, axial and sagittal levels (FIG. 24). The accumulation of [¹¹C]1 clearly delineates the mGluR2-rich regions in the rat brain. Time-activity curves (TACs) showed fast radioactivity uptake (SUVmax=2.3) and time-dependent accumulation of radioactivity in different brain regions. The highest accumulation of [¹¹C]1 was in the thalamus, followed by striatum, cerebellum, and cortex. (FIG. 25). Blocking studies were conducted to investigate specificity and selectivity of [C]1 for mGluR2. Pretreatment with the structurally distinct in vivo active mGluR2 PAM ligand 3 (4 mg/kg i.v.) 10 min before [¹¹C]1 injection resulted in a 28-37% decrease of [¹¹C]1 uptake in different brain areas at the 10-30 min time window (FIG. 22). On the other hand, administration of unlabeled compound 1, using a dose of 4 mg/kg iv. 10 min before [¹¹C]1 injection, resulted in a 33-49% enhancement of radioactivity uptake in the different brain areas at the same time window as mentioned above. These results confirm that [¹¹C]1 has in vivo specific binding to mGluR2 in the rat brain. The significant increase of radioactivity uptake after self-blocking indicates that the compound 1, as a mGluR2 PAM, is capable of potentiating strong pharmacological effects, making 1 a valuable therapeutic agent.

### CONCLUSION

mGluR2 has been implicated in various neurological conditions, such as anxiety, drug abuse and schizophrenia. Development of mGluR2-specific PET radioligands is imperative to investigate mGluR2 function. In 2012, LY2140023, as mGluR2/3 agonist, failed in phase III clinical trials for the treatment of schizophrenia due to the mGluR3 binding, highlighting the importance of mGluR2 specificity in determining mGluR2-related therapeutic outcomes [see refs. 64, 65]. To avoid the off-target binding toward mGluR3 and other mGlu receptors, mGluR2 PAMs within the present claims were studied as PET imaging candidates with a focus on the benzimidazole derivatives. Three benzimidazole derivatives were made as candidates for PET imaging of mGluR2. In general, syntheses of all compounds was straightforward with good yield. The radiolabeling was done with [¹¹C]CH₃I via one-step O-methylation of the phenol precursor. Compounds 1, 2, and 7 were evaluated for the molecular binding modes, pharmacology, physicochemical properties and BBB permeability before in vivo characterization to avoid the low, non-specific brain uptake issues among the most reported mGluR2 PET tracers. Compounds 1, 2, and 7 showed similar binding results in the molecular modeling experiments with compound 1 exhibiting the most favorable binding profile. In the cAMP assay, compound 1 showed the highest mGluR2 binding potency and excellent selectivity against other mGlu receptors, and so did compound 2. The log P values of compounds 1, 2, and 7 varied from 3.30 to 3.86 in the range of preferred lipophilicity. Compounds 1, 2, and 7 exhibited good stability and protein binding profiles.

The pharmacokinetic characterization of [¹¹C]1 from ex vivo whole body biodistribution experiments revealed that [¹¹C]1 was a CNS penetrant and had a pharmacokinetic profile that is suitable as brain PET radioligand. In vivo PET imaging confirmed the BBB permeability of [¹¹C]1 with a maximum SUV value of 2.3 at thalamus at 2 min post-injection. Blockade of 28-37% of [¹¹C]1 brain uptake was achieved by pre-administration of a structurally distinct mGluR2 PAM 3 (JNJ-46356479). The self-blocking experiment with compound 1, however, revealed an unexpected radioactivity enhancement by 33-49%, which was not seen in the other reported mGluR2 PAM radioligands. The demonstrated strong potentiating effect of the compounds of the present examples provides a therapeutic possibility for the treatment of mGluR2-related diseases.

In sum, benzimidazole derivatives were synthesized and characterized as mGluR2 PAMs. Compound 1 demonstrated nanomolar binding potency toward mGluR2 and excellent selectivity over other mGluRs. Further in vitro pharmacological and brain permeability evaluations confirmed the potential of compound 1 as PET imaging ligand. A robust and reliable one-step radiosynthetic procedure was established for radiolabeling compound 1 with carbon-11. The desired product [¹¹C]1 was obtained with a radiochemical yield of 20±2% (n=10, decay-corrected) based on [¹¹C]C02 and a molar activity of 98±30 GBq/μmol at the end of synthesis (50 min). The ex vivo pharmacokinetic results of [¹¹C]1 suggested its reversible accumulation in most tissue areas and hepatobiliary & urinary excretions feature. PET imaging studies indicated that [¹¹C]1 crossed the BBB rapidly and was mainly accumulated in the mGluR2-rich regions of the rat brain such as the thalamus, cerebellum, striatum and cortex. The blocking studies using mGluR2-selective PAM 3 significantly reduced the [¹¹C]1 uptake in these brain regions, indicating the high and specific uptake of [¹¹C]1 in rat brains. Distinct from previous observations of mGluR2 PET radioligands, self-blocking of [¹¹C]1 resulted in an apparent uptake increase in the accumulation by almost 50%. This result indicates a significant modulation effect of compound 1 in vivo as mGluR2 PAM, which bears promising therapeutic applications for translational studies in neurological conditions and/or disorders. Altogether, these results suggest that [¹¹C]1 and other compounds within the present claims is a suitable PET imaging candidate for mGluR2 in rat brains.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

[1] Y. Tanabe, et al, A family of metabotropic glutamate receptors, Neuron 8 (1992) 169-179.
[2] J. Cartmell, et al, Regulation of neurotransmitter release by metabotropic glutamate receptors, J. Neurochem. 75 (2000) 889-907.
[3] G. Gu, et al, Distribution of metabotropic glutamate 2 and 3 receptors in the rat forebrain: Implication in emotional responses and central disinhibition, Brain Res. 1197 (2008) 47-62.
[4] R. A. Wright, et al, CNS distribution of metabotropic glutamate 2 and 3 receptors: transgenic mice and [³H]LY459477 autoradiography, Neuropharmacology, 66 (2013) 89-98.
[5] S. Yin, et al, Selective actions of novel allosteric modulators reveal functional heteromers of metabotropic glutamate receptors in the CNS, J. Neurosci. 34 (2014) 79-94.
[6] S. H. Kim, et al, Group II metabotropic glutamate receptor stimulation triggers production and release of Alzheimer's amyloid OE≤42 from isolated intact nerve terminals, Journal of Neuroscience 30 (2010) 3870-3875.
[7] G. Richards, et al, Altered distribution of mGlu2 receptors in β-amyloid-affected brain regions of Alzheimer cases and aged PS2APP mice, Brain Res. 1363 (2010) 180-190.
[8] H.-g. Lee, et al, Aberrant expression of metabotropic glutamate receptor 2 in the vulnerable neurons of Alzheimer's disease, Acta Neuropathol. 107 (2004) 365-371.
[9] F. Caraci, et al, Targeting group II metabotropic glutamate (mGlu) receptors for the treatment of psychosis associated with Alzheimer's disease: selective activation of mGlu2 receptors amplifies-amyloid toxicity in cultured neurons, whereas dual activation of mGlu2 and mGlu3 receptors is neuroprotective, Mol. Pharmacol. 79 (2011) 618-626.
[10] J. L. Moreno, et al, Group II metabotropic glutamate receptors and schizophrenia, Cell. Mol.

Life Sci. 66 (2009) 3777-3785.
[11] S. Chaki, Group II metabotropic glutamate receptor agonists as a potential drug for schizophrenia, Eur. J. Pharmacol. 639 (2010) 59-66.
[12] A. M. Downing, et al, A double-bind, placebo-controlled comparator study of LY2140023 monohydrate in patients with schizophrenia, BMC Psychiatry 14 (2014) 351/351-351/324.
[13] S. Patil, et al, Activation of mGluR2/3 receptors as a new approach to treat schizophrenia: a randomized Phase 2 clinical trial., Nat. Med. 13 (2007) 1102-1107.
[14] A. M. Feyissa, et al, Elevated level of metabotropic glutamate receptor 2/3 in the prefrontal cortex in major depression, Prog. Neuropsychopharmacol. Biol. Psychiatry 34 (2010) 279-283.
[15] M. P. Johnson, et al, Metabotropic glutamate 2 receptor potentiators: receptor modulation, frequency-dependent synaptic activity, and efficacy in preclinical anxiety and psychosis model(s), Psychopharmacology (Berl) 179 (2005) 271-283.
[16] M. Mazzitelli, et al, Group II Metabotropic Glutamate Receptors: Role in Pain Mechanisms and Pain Modulation, Front. Mol. Neurosci. 11 (2018) 383.
[17] S. Chiechio, et al, Transcriptional regulation of type-2 metabotropic glutamate receptors: an epigenetic path to novel treatments for chronic pain, Trends Pharmacol. Sci. 31 (2010) 153-160.
[18] C. M. Niswender, et al, Metabotropic glutamate receptors: physiology, pharmacology, and disease, Annu. Rev. Pharmacol. Toxicol. 50 (2010) 295-322.
[19] M. Yu, et al, Radiolabeling and biodistribution of methyl 2-(methoxycarbonyl)-2-(methylamino) bicyclo [2.1.1]-hexane-5-carboxylate, a potent neuroprotective drug, Life Sci. 73 (2003) 1577-1585.
[20] J.-Q. Wang, et al, Radiosynthesis of PET radiotracer as a prodrug for imaging group II metabotropic glutamate receptors in vivo, Bioorg. Med. Chem. Lett. 22 (2012) 1958-1962.
[21] Z. Zhang, et al, Imaging of metabotropic glutamate receptors (mGluRs), Neuroimaging-clinical applications, InTech-Open Access Publisher, Rijeka, Croatia, (2012) pp. 499-532.
[22] A. A. Trabanco, et al, Progress in the development of positive allosteric modulators of the metabotropic glutamate receptor 2, Curr. Med. Chem. 18 (2011) 47-68.
[23] D. J. Sheffler, et al, Recent Progress in the Synthesis and Characterization of Group II Metabotropic Glutamate Receptor Allosteric Modulators, ACS Chem. Neurosci. 2 (2011) 382-393.
[24] J.-I. Andres, et al, Synthesis, Evaluation, and Radiolabeling of New Potent Positive Allosteric Modulators of the Metabotropic Glutamate Receptor 2 as Potential Tracers for Positron Emission Tomography Imaging, J. Med. Chem. 55 (2012) 8685-8699.
[25] G. Leurquin-Sterk, et al, What We Observe In Vivo Is Not Always What We See In Vitro: Development and Validation of 11C-JNJ-42491293, A Novel Radioligand for mGluR2, J. Nucl. Med. 58 (2017) 110-116.
[26] T. Lohith, et al, First-in-human PET imaging of mGluR2 receptors, J. Nucl. Med. 57 (2016) 1.
[27] P. McQuade et al, Discovery and preclinical evaluation of an mGluR2-NAM PET radioligand, J. Nucl. Med. 57 (2016) 1.
[28] V. Majo, et al, Development of a [18F]-labeled positive allosteric modulator of the metabotropic glutamate receptor 2 (mGluR2) as a potential PET tracer, J. Nucl. Med. 54 (2016) 1.
[29] Y. Ma, et al, Synthesis and evaluation of 1-(cyclopropylmethyl)-4-(4-[¹¹C]methoxyphenyl)-piperidin-1-yl-2-oxo-1,2-dihydropyridine-3-carbonitrile ([¹¹C]CMDC) for PET imaging of metabotropic glutamate receptor 2 in the rat brain, Bioorg. Med. Chem. 25 (2017) 1014-1021.
[30] K. Kumata, et al, Synthesis and in vitro evaluation of three novel radiotracers for imaging of metabotropic glutamate receptor subtype 2 in rat brain, Bioorg. Med. Chem. Lett. 27 (2017) 5.
[31] X. Zhang, et al, Synthesis and Preliminary Studies of a Novel Negative Allosteric Modulator, 7-((2,5-Dioxopyrrolidin-1-yl)methyl)-4-(2-fluoro-4-[(¹¹)C]methoxyphenyl) quinoline-2-carboxamide, for Imaging of Metabotropic Glutamate Receptor 2, ACS Chem. Neurosci. 8 (2017) 1937-1948.
[32] K. Kumata, et al, Synthesis and evaluation of 4-(2-fluoro-4-[(¹¹)C]methoxyphenyl)-5-((2-methylpyridin-4-yl)methoxy)picolinamide for PET imaging of the metabotropic glutamate receptor 2 in the rat brain, Bioorg. Med. Chem. 27 (2019) 483-491.
[33] Z. Li, et al, Radiotracer compounds, WO2016033190 (A1) (2016) 1-83.
[34] M. L. M. Van Gool, et al, Radiolabeled mGluR2 PET ligands, WO2016087489 (A1) (2016) 1-80.
[35] P. L. D'Alessandro, et al, The identification of structurally novel, selective, orally bioavailable positive modulators of mGluR2, Bioorg. Med. Chem. Lett. 20 (2010) 759-762.
[36] I. V. Efremov, et al, Azabenzimidazolyl compounds as potentiators of mGluR2 subtype of glutamate receptor and their preparation, pharmaceutical compositions and use in the treatment of diseases, WO2008012622 (2008) 1-144.
[37] L. Zhang, et al, 1-[(1-Methyl-1H-imidazol-2-yl)methyl]-4-phenylpiperidines as mGluR2 Positive Allosteric Modulators for the Treatment of Psychosis, J. Med. Chem. 54 (2011) 1724-1739.
[38] L. Zhang, et al, 3-(Imidazolylmethyl)-3-aza-bicyclo[3.1.0]hexan-6-yl)methyl ethers: A novel series of mGluR2 positive allosteric modulators, Bioorga. Med. Chem. Lett. 18 (2008) 5493-5496.
[39] J. M. Cid, et al, Discovery of 8-Trifluoromethyl-3-cyclopropylmethyl-7-[(4-(2,4-difluorophenyl)-1-piperazinyl)methyl]-1,2,4-triazolo[4,3-a]pyridine (JNJ-46356479), a Selective and Orally Bioavailable mGlu2 Receptor Positive Allosteric Modulator (PAM), J. Med. Chem. 59 (2016) 8495-8507.
[40] E. Krieger, et al, Improving physical realism, stereochemistry, and side-chain accuracy in homology modeling: Four approaches that performed well in CASP8, Proteins 77 (2009) 114-122.
[41] L. J. McGuffin, et al, Accurate template-based modeling in CASP12 using the IntFOLD4-TS, ModFOLD6, and ReFOLD methods, Proteins 86 (2018) 335-344.
[42] A. Waterhouse, et al, SWISS-MODEL: homology modelling of protein structures and complexes, Nucleic Acids Res. 46 (2018) W296-W303.
[43] D. Eisenberg, et al, VERIFY3D: assessment of protein models with three-dimensional profiles, Methods Enzymol. 277 (1997) 396-404.
[44] C. Colovos, et al, Verification of protein structures: patterns of nonbonded atomic interactions, Protein Sci. 2 (1993) 1511-1519.
[45] W. X. Tong, et al, Partial Order Optimum Likelihood (POOL): Maximum Likelihood Prediction of Protein Active Site Residues Using 3D Structure and Sequence Properties, PLoS Comput.

Biol. 5 (2009).
[46] K. P. Tan, et al, DEPTH: a web server to compute depth and predict small-molecule binding cavities in proteins, Nucleic Acids Res. 39 (2011) W242-W248.
[47] B. D. Huang, MetaPocket: A Meta Approach to Improve Protein Ligand Binding Site Prediction, OMICS 13 (2009) 325-330.
[48] G. M. Morris, et al, Automated docking using a Lamarckian genetic algorithm and an empirical binding free energy function, J. Comput. Chem. 19 (1998) 1639-1662.
[49] L. Perez-Benito, et al, Molecular Switches of Allosteric Modulation of the Metabotropic Glutamate 2 Receptor, Structure 25 (2017) 1153-1162 e1154.
[50] M. L. J. Doornbos, et al, Discovery and Kinetic Profiling of 7-Aryl-1,2,4-triazolo[4,3-a]pyridines: Positive Allosteric Modulators of the Metabotropic Glutamate Receptor 2, J. Med. Chem. 60 (2017) 6704-6720.
[51] B. L. Roth, Assay protocol book, in: N. PDSP (Ed.), Department of Pharmacology, University of North Carolina at Chapel Hill, 2018.
[52] J. O. DiRaddo, et al, A real-time method for measuring cAMP production modulated by Gαi/o-coupled metabotropic glutamate receptors, J. Pharmacol. Exp. Ther. 349 (2014) 373-382, 310 pp.
[53] X. Jin, et al, The mGluR2 Positive Allosteric Modulator BINA Decreases Cocaine Self-Administration and Cue-Induced Cocaine-Seeking and Counteracts Cocaine-Induced Enhancement of Brain Reward Function in Rats, Neuropsychopharmacol. 35 (2010) 2021-2036.
[54] D. J. Minick, et al, A comprehensive method for determining hydrophobicity constants by reversed-phase high-performance liquid chromatography, J. Med. Chem. 31 (1988) 1923-1933.
[55] V. W. Pike, PET radiotracers: crossing the blood-brain barrier and surviving metabolism, Trends Pharmacol. Sci. 30 (2009) 431-440.
[56] M. J. Banker, et al, Development and validation of a 96-well equilibrium dialysis apparatus for measuring plasma protein binding, J. Pharm. Sci. 92 (2003) 967-974.
[57] L. Di, et al, Development and application of high throughput plasma stability assay for drug discovery, International Journal of Pharmaceutics, 297 (2005) 110-119.
[58] J. B. Houston, Utility of in vitro drug metabolism data in predicting in vivo metabolic clearance, Biochem. Pharmacol. 47 (1994) 1469-1479.
[59] D. W. Engers, et al, Synthesis and evaluation of a series of heterobiarylamides that are centrally penetrant metabotropic glutamate receptor 4 (mGluR4) positive allosteric modulators (PAMs), J. Med. Chem. 52 (2009) 4.
[60] K.-E. Kil, et al, Radiosynthesis of N-(4-chloro-3-[¹¹C]methoxyphenyl)-2-picolinamide ([¹¹C]ML128) as a PET radiotracer for metabotropic glutamate receptor subtype 4 (mGlu4), Bioorg. Med. Chem. 21 (2013) 5955-5962.
[61] L. Di, et al, Development and application of an automated solution stability assay for drug discovery, J. Biomol. Screen. 11 (2006) 40-47.
[62] L. Di, et al, High throughput artificial membrane permeability assay for blood-brain barrier, Eur. J. Med. Chem. 38 (2003) 223-232.
[63] Promega, Pgp-Glo Assay System, Instructions for use of products V3591 and V3601, Promega, 2015.
[64] M. J. Fell, et al, Evidence for the role of metabotropic glutamate (mGlu)2 not mGlu3 receptors in the preclinical antipsychotic pharmacology of the mGlu2/3 receptor agonist (−)-(1R,4S,5S,6S)-4-amino-2-sulfonylbicyclo[3.1.0]hexane-4,6-dicarboxylic acid (LY404039), J. Pharmacol. Exp. Ther. 326 (2008) 209-217.
[65] M. L. Woolley, et al, The mGlu2 but not the mGlu3 receptor mediates the actions of the mGluR2/3 agonist, LY379268, in mouse models predictive of antipsychotic activity, Psychopharmacology (Berl) 196 (2008) 431-440.
[66] S. F. Altschul, et al, Gapped BLAST and PSI-BLAST: a new generation of protein database search programs, Nucleic Acids Res. 25 (1997) 3389-3402.
[67] N. Kunishima, et al, Structural basis of glutamate recognition by a dimeric metabotropic glutamate receptor, Nature 407 (2000) 971-977.
[68] E. Dobrovetsky, et al, Ligand Binding Domain of Metabotropoc glutamate receptor mGluR5 complexed with glutamate, (2017).
[69] A. Koehl, et al, Structural insights into the activation of metabotropic glutamate receptors, Nature 566 (2019) 79-84.
[70] M. D. Hanwell, et al, Avogadro: an advanced semantic chemical editor, visualization, and analysis platform, J. Cheminform. 4 (2012) 17.
[71] E. Krieger, et al, Improving physical realism, stereochemistry, and side-chain accuracy in homology modeling: Four approaches that performed well in CASP8, Proteins, 77 Suppl 9 (2009) 114-122.
[72] N. Kunishima, et al, Structural basis of glutamate recognition by a dimeric metabotropic glutamate receptor, Nature, 407 (2000) 971-977.
[73] E. Dobrovetsky, et al, Ligand Binding Domain of Metabotropoc glutamate receptor mGluR5 complexed with glutamate, (2017).
[74] A. Koehl, et al, Structural insights into the activation of metabotropic glutamate receptors, Nature, 566 (2019) 79.
[75] S. F. Altschul, et al, Gapped BLAST and PSI-BLAST: a new generation of protein database search programs, Nucleic Acids Research, 25 (1997) 3389-3402.
[76] L. J. McGuffin, et al, Accurate template-based modeling in CASP12 using the IntFOLD4-TS, ModFOLD6, and ReFOLD methods, Proteins-Structure Function and Bioinformatics, 86 (2018) 335-344.
[77] D. Eisenberg, et al, VERIFY3D: assessment of protein models with three-dimensional profiles, Methods Enzymol, 277 (1997) 396-404.
[78] A. Waterhouse, et al, SWISS-MODEL: homology modelling of protein structures and complexes, Nucleic Acids Res, 46 (2018) W296-W303.
[79] C. Colovos, et al, Verification of protein structures: patterns of nonbonded atomic interactions, Protein Sci, 2 (1993) 1511-1519.
[80] M. L. J. Doornbos, et al, Discovery and Kinetic Profiling of 7-Aryl-1,2,4-triazolo[4,3-a]pyridines: Positive Allosteric Modulators of the Metabotropic Glutamate Receptor 2, J Med Chem, 60 (2017) 6704-6720.
[81] W. X. Tong, et al, Partial Order Optimum Likelihood (POOL): Maximum Likelihood Prediction of Protein Active Site Residues Using 3D Structure and Sequence Properties, Plos Computational Biology, 5 (2009).
[82] K. P. Tan, et al, DEPTH: a web server to compute depth and predict small-molecule binding cavities in proteins, Nucleic Acids Research, 39 (2011) W242-W248.
[83] B. D. Huang, MetaPocket: A Meta Approach to Improve Protein Ligand Binding Site Prediction, Omics, 13 (2009) 325-330.
[84] B. L. Roth, Assay protocol book, in: N. PDSP (Ed.), Department of Pharmacology, University of North Carolina at Chapel Hill, 2018.
[85] J. O. DiRaddo, et al, A real-time method for measuring cAMP production modulated by Gai/o-coupled metabotropic glutamate receptors, J. Pharmacol. Exp. Ther., 349 (2014) 373-382, 310 pp.
[86] D. J. Minick, et al, A comprehensive method for determining hydrophobicity constants by reversed-phase high-performance liquid chromatography, Journal of Medicinal Chemistry, 31 (1988) 1923-1933.
[87] M. J. Banker, et al, Development and validation of a 96-well equilibrium dialysis apparatus for measuring plasma protein binding, Journal of Pharmaceutical Sciences, 92 (2003) 967-974.
[88] L. Di, et al, Development and application of high throughput plasma stability assay for drug discovery, International Journal of Pharmaceutics, 297 (2005) 110-119.
[89] J. B. Houston, Utility of in vitro drug metabolism data in predicting in vivo metabolic clearance, Biochemical Pharmacology, 47 (1994) 1469-1479.
[90] L. Di, et al, Development and application of an automated solution stability assay for drug discovery, Journal of Biomolecular Screening, 11 (2006) 40-47.

1. A compound of Formula (I): 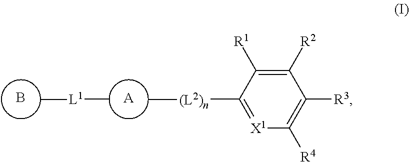
or a pharmaceutically acceptable salt thereof, wherein:
ring B is selected from formula (i), formula (ii), formula (v), and formula (vi): 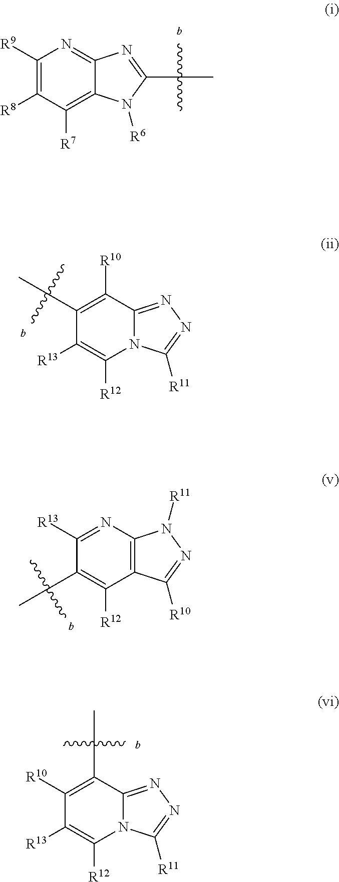
wherein b indicates a point of attachment of ring B to L¹;
L¹ is C₁₋₃ alkylene, which is optionally substituted with 1 or 2 substituents independently selected from oxo, halo, C₁₋₃ haloalkyl, OH, C₁₋₃ alkoxy, C₁₋₃ haloalkoxy, amino, C₁₋₆ alkylamino, di(C₁₋₆ alkyl)amino, thio, and C₁₋₆ alkylthio;
ring A is selected from formula (iii) and formula (iv): 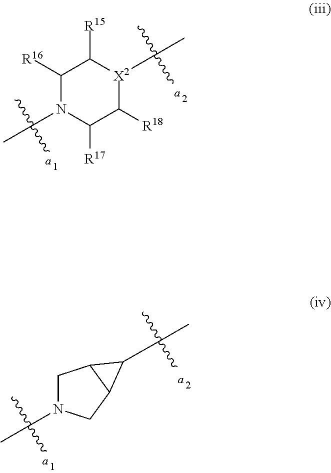
wherein a₁ indicates a point of attachment of ring A to L¹, and a₂ indicates a point of attachment of ring A to L²;
each L² is independently selected from C₁₋₃ alkylene, O, N(R^{N}), and S(═O)₂, wherein said C₁₋₃ alkylene is optionally substituted with 1 or 2 substituents independently selected from halo, C₁₋₃ haloalkyl, OH, C₁₋₃ alkoxy, C₁₋₃ haloalkoxy, amino, C₁₋₆ alkylamino, di(C₁₋₆ alkyl)amino, thio, and C₁₋₆ alkylthio;
each R^{N} is selected from H and C₁₋₃ alkyl;
n is 0, 1, 2, or 3;
X¹ is selected from N and CR⁵;
X² is selected from N and CR¹⁴; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆ alkylamino, di(C₁₋₆ alkyl)amino, thio, and C₁₋₆ alkylthio;
or R⁶ and R⁷, together with the carbon atom to which R⁷ is attached and the N atom to which R⁶ is attached form a 5-7-membered heterocycloalkyl ring, which is optionally substituted with 1 or 2 substituents independently selected from OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆ alkylamino, di(C₁₋₆ alkyl)amino, thio, and C₁₋₆ alkylthio;
provided that the compound of Formula (I) comprises at least one radioisotope selected from ¹¹C and ¹⁸F,
and further provided that the compound of Formula (I) is not any of the following compounds:

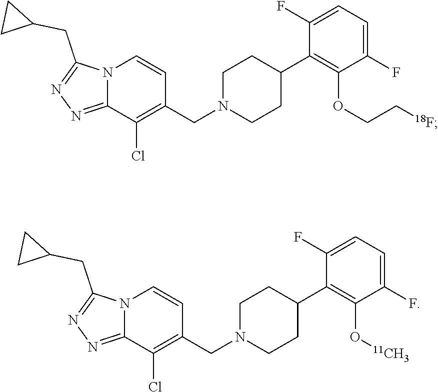

2. The compound of claim 1, wherein:
L¹ is C₁₋₃ alkylene;
each L² is independently selected from C₁₋₃ alkylene, O, and N(R^{N});
n is 0, 1, or 2; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, halo, C₁₋₃ alkyl, C₁₋₃ alkoxy, C₁₋₃ haloalkyl, C₁₋₃ haloalkoxy, and C₃₋₁₀ cycloalkyl-C₁₋₃ alkyl.

3. The compound of claim 1, wherein the compound of Formula (I) has formula: 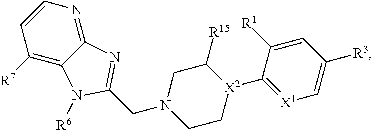
or a pharmaceutically acceptable salt thereof, wherein:
R¹⁵ is selected from H and C₁₋₃ alkyl;
X¹ is selected from N and CH;
X² is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl, C₁₋₃ haloalkyl, or HO—C₁₋₃ alkyl; and
R⁷ is H, or R⁷ and R⁶ together with the atoms to which they are attached form a 6-membered heterocycloalkyl ring.

4. The compound of claim 3, wherein the compound is selected from: 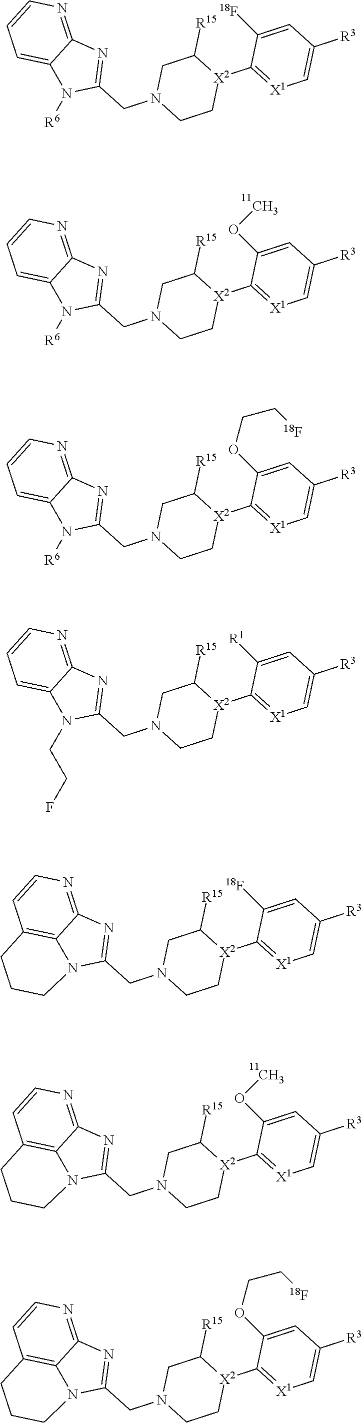
or a pharmaceutically acceptable salt thereof.

5. The compound of claim 1, wherein the compound of Formula (I) has formula: 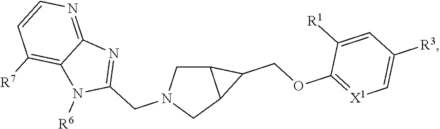
or a pharmaceutically acceptable salt thereof, wherein:
X¹ is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl; and
R⁷ is H, or R⁷ and R⁶ together with the atoms to which they are attached form a 6-membered heterocycloalkyl ring.

6. The compound of claim 5, wherein the compound is selected from: 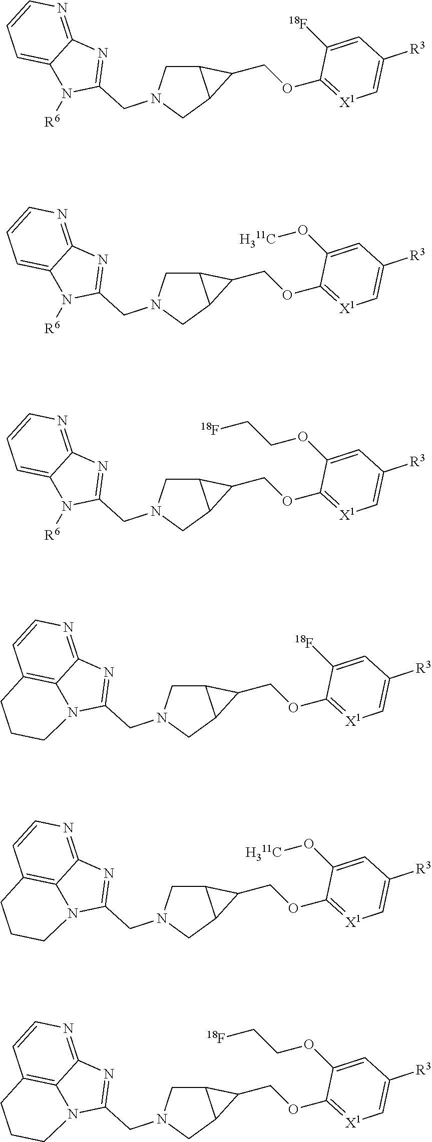
or a pharmaceutically acceptable salt thereof.

7. The compound of claim 1, wherein the compound of Formula (I) has formula: 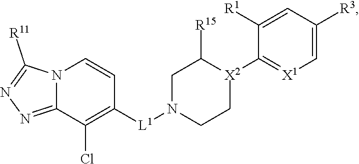
or a pharmaceutically acceptable salt thereof, wherein:
L¹ is selected from CH₂ and C(═O);
R¹¹ is C₃₋₁₀ cycloalkyl-C₁₋₃ alkyl;
R¹⁰ is selected from halo and C₁₋₃ haloalkyl;
R¹⁵ is selected from H and C₁₋₃ alkyl;
X¹ is selected from N and CH;
X² is selected from N and CH; and
R¹ and R³ are each independently selected from halo, C₁₋₃ alkoxy, C₁₋₃ haloalkyl, and C₁₋₃ haloalkoxy.

8. The compound of claim 7, wherein the compound is selected from: 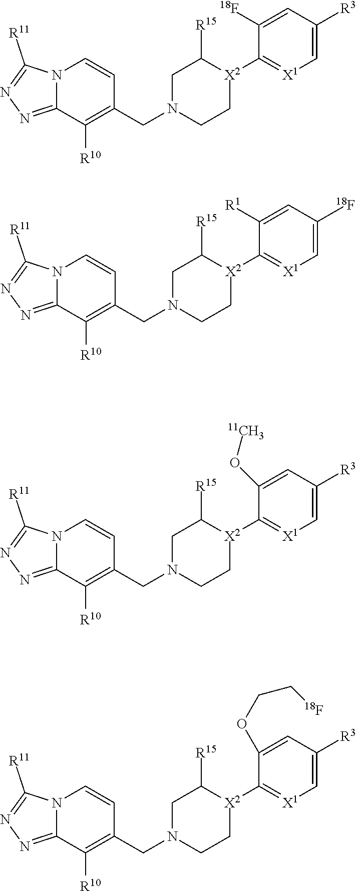
or a pharmaceutically acceptable salt thereof.

9. The compound of claim 1, wherein the compound is selected from: 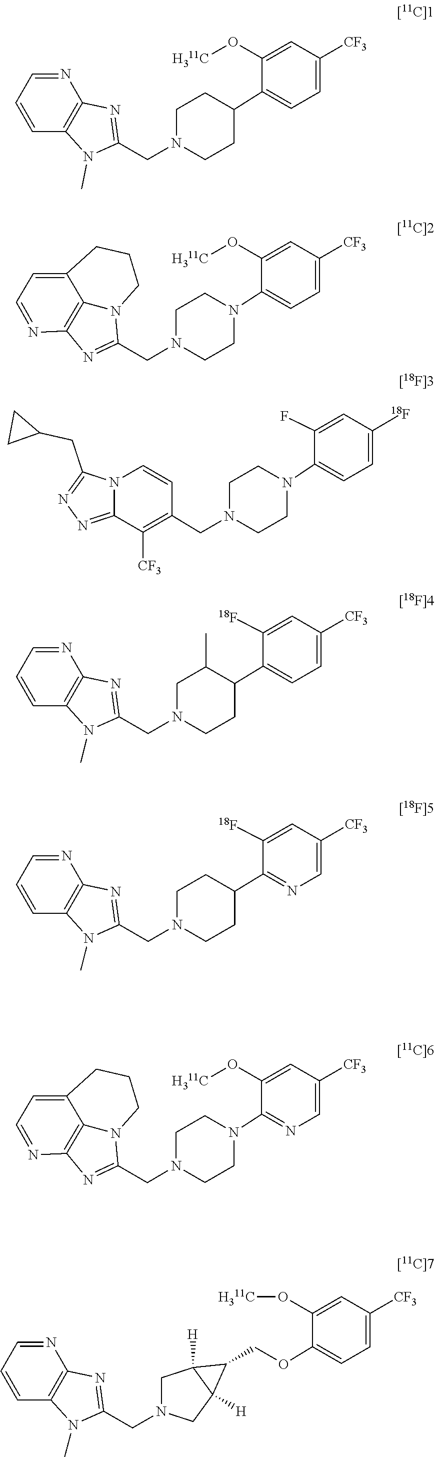 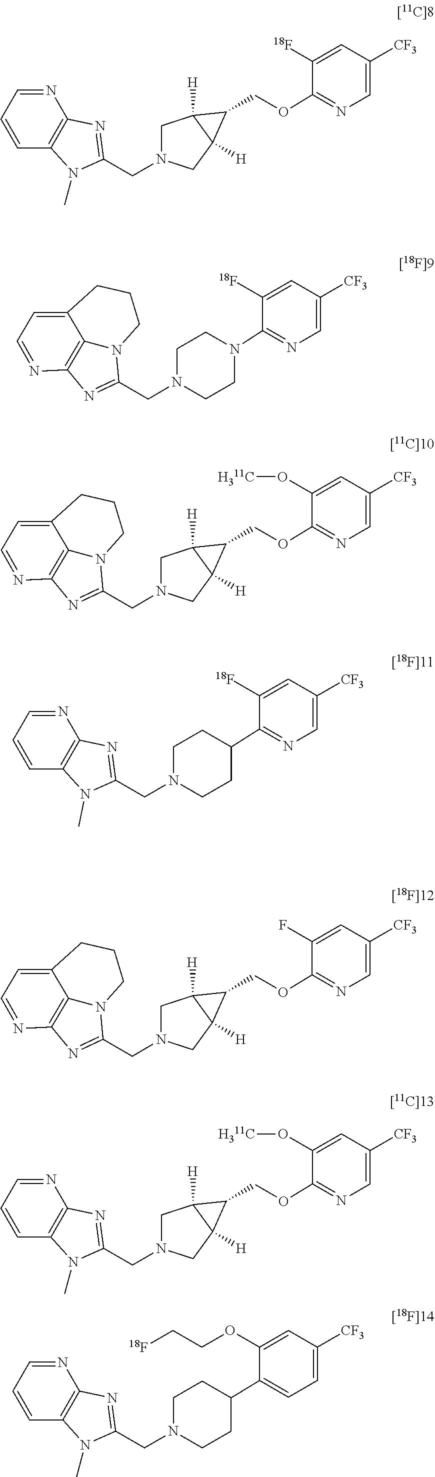 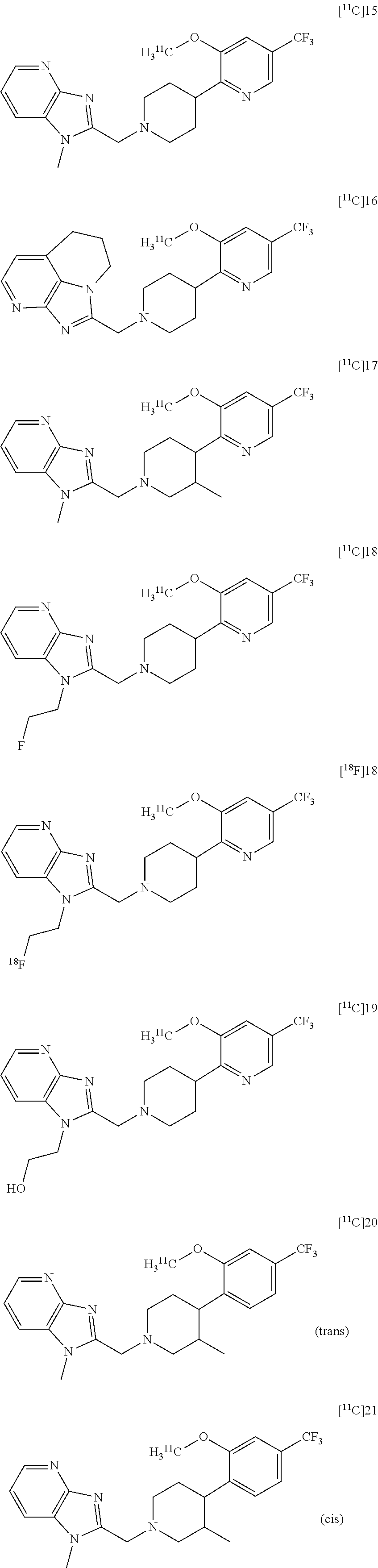 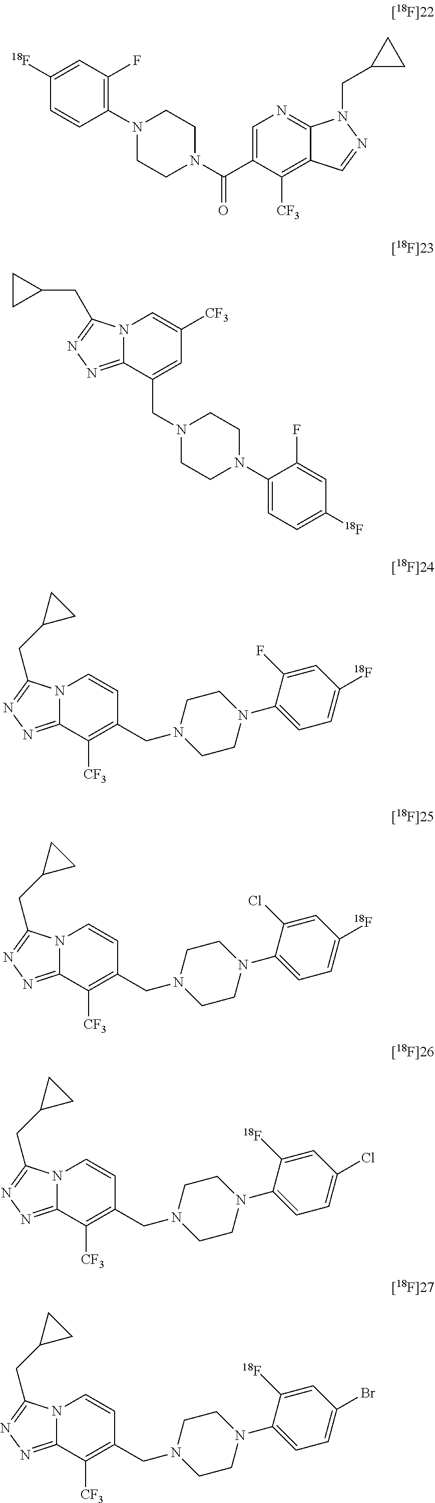 [SMILES: O=C(c1cnc2c(cnn2CC2CC2)c1C(F)(F)F)N1CCN(c2ccc([18F])cc2F)CC1 ; Fc1cc([18F])ccc1N1CCN(Cc2cc(C(F)(F)F)cn3c(CC4CC4)nnc23)CC1 ; Fc1cc([18F])ccc1N1CCN(Cc2ccn3c(CC4CC4)nnc3c2C(F)(F)F)CC1 ; FC(F)(F)c1c(CN2CCN(c3ccc([18F])cc3Cl)CC2)ccn2c(CC3CC3)nnc12 ; FC(F)(F)c1c(CN2CCN(c3ccc(Cl)cc3[18F])CC2)ccn2c(CC3CC3)nnc12 ; FC(F)(F)c1c(CN2CCN(c3ccc(Br)cc3[18F])CC2)ccn2c(CC3CC3)nnc12] 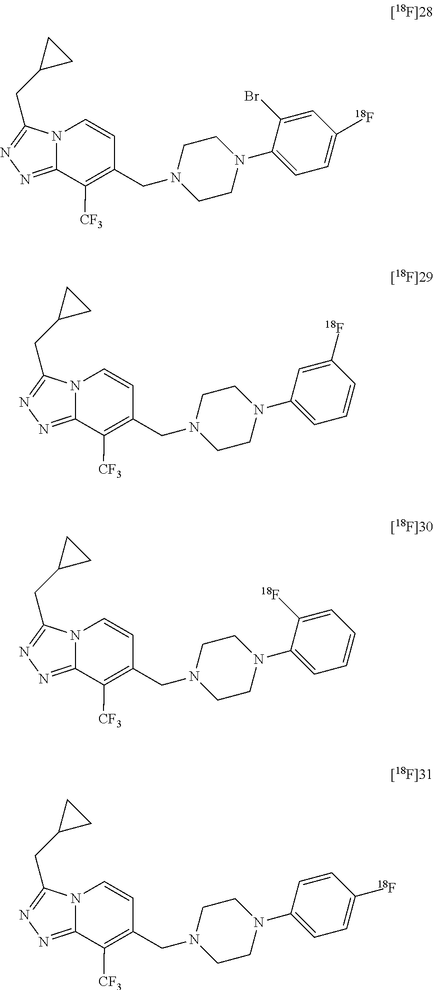 [SMILES: FC(F)(F)c1c(CN2CCN(c3ccc([18F])cc3Br)CC2)ccn2c(CC3CC3)nnc12 ; FC(F)(F)c1c(CN2CCN(c3cccc([18F])c3)CC2)ccn2c(CC3CC3)nnc12 ; FC(F)(F)c1c(CN2CCN(c3ccccc3[18F])CC2)ccn2c(CC3CC3)nnc12 ; FC(F)(F)c1c(CN2CCN(c3ccc([18F])cc3)CC2)ccn2c(CC3CC3)nnc12]
or a pharmaceutically acceptable salt thereof.

10. A pharmaceutical composition comprising a compound of claim 1, or a pharmaceutically acceptable salt thereof, and a pharmaceutically acceptable carrier.

11. A method of imaging a brain of a subject, the method comprising:
i) administering to the subject an effective amount of a compound of claim 1 or a pharmaceutically acceptable salt thereof;
ii) waiting a time sufficient to allow the compound to accumulate in the brain to be imaged; and
iii) imaging the brain with an imaging technique.

12. A method of monitoring treatment of a psychiatric or a neurological disorder associated with mGluR2 in a subject, the method comprising:
i) administering to the subject an effective amount of a compound of claim 1, or a pharmaceutically acceptable salt thereof;
ii) waiting a time sufficient to allow the compound to accumulate in a brain of the subject;
iii) imaging the brain of the subject with an imaging technique;
iv) administering to the subject a therapeutic agent in an effective amount to treat the psychiatric or the neurological disorder;
v) after iv), administering to the subject a second effective amount of the compound, or a pharmaceutically acceptable salt thereof;
vi) waiting a time sufficient to allow the compound to accumulate in the brain of the subject;
vii) imaging the brain of the subject with an imaging technique; and
viii) comparing the image of step iii) and the image of step vii).

13. The method of or claim 12, wherein the imaging technique is selected from positron emission tomography (PET) imaging, positron emission tomography with computer tomography (PET/CT) imaging, and positron emission tomography with magnetic resonance (PET/MRI) imaging.

14. The method of claim 12, wherein the neurological disorder associated with mGluR2 is selected from Alzheimer's disease, Parkinson's disease, dyskinesia, Lewy body disease, Prion disease, motor neuron disease (MND), and Huntington's disease.

15. The method of claim 12, wherein the psychiatric disorder associated with mGluR2 is selected from schizophrenia, psychosis, anxiety, depression, drug abuse, pain, smoking cessation, and epilepsy.

16. A compound of Formula (II) 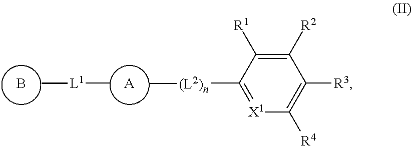
or a pharmaceutically acceptable salt thereof, wherein:
ring B is selected from formula (i), formula (ii), formula (v), and formula (vi): 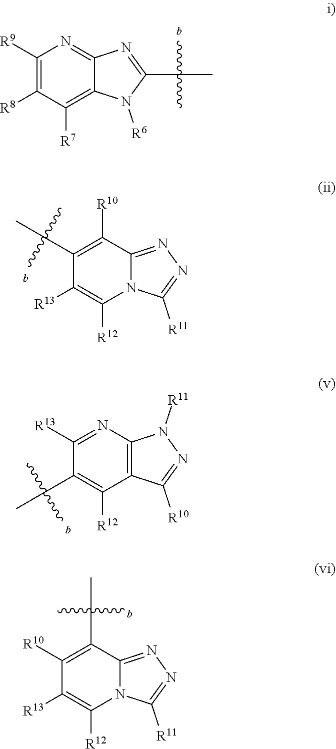
wherein b indicates a point of attachment of ring B to L¹;
L¹ is C₁₋₃ alkylene, which is optionally substituted with 1 or 2 substituents independently selected from halo, C₁₋₃ haloalkyl, OH, C₁₋₃ alkoxy, C₁₋₃ haloalkoxy, amino, C₁₋₆ alkylamino, di(C₁₋₆ alkyl)amino, thio, and C₁₋₆ alkylthio;
ring A is selected from formula (iii) and formula (iv): 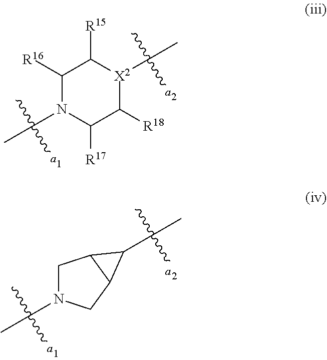
wherein a₁ indicates a point of attachment of ring A to L¹, and a₂ indicates a point of attachment of ring A to L²;
each L² is independently selected from C₁₋₃ alkylene, O, N(R^{N}), and S(═O)₂, wherein said C₁₋₃ alkylene is optionally substituted with 1 or 2 substituents independently selected from halo, C₁₋₃ haloalkyl, OH, C₁₋₃ alkoxy, C₁₋₃ haloalkoxy, amino, C₁₋₆ alkylamino, di(C₁₋₆ alkyl)amino, thio, and C₁₋₆ alkylthio;
n is 0, 1, 2, or 3;
each R^{N} is selected from H and C₁₋₃ alkyl;
X¹ is selected from N and CR⁵;
X² is selected from N and CR¹⁴;
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆ alkylamino, di(C₁₋₆ alkyl)amino, thio, and C₁₋₆ alkylthio; and
or R⁶ and R⁷, together with the carbon atom to which R⁷ is attached and the N atom to which R⁶ is attached form a 5-7-membered heterocycloalkyl ring, which is optionally substituted with 1 or 2 substituents independently selected from OH, SH, NO₂, CN, halo, C₁₋₃ alkyl, C₁₋₃ alkoxy, C₁₋₃ alkylthio, C₁₋₃ haloalkyl, C₁₋₃ haloalkoxy, cyano-C₁₋₃ alkyl, HO—C₁₋₃ alkyl, C₃₋₁₀ cycloalkyl-C₁₋₃ alkyl, amino, C₁₋₆ alkylamino, di(C₁₋₆ alkyl)amino, thio, and C₁₋₆ alkylthio;
provided that:
(a) if the ring B has formula (i) and X² is CR¹⁴, then X¹ is N or R¹ is C₁₋₃ haloalkoxy; and
(b) if the ring B has formula (ii), then X¹ is N.

17. The compound of claim 16, wherein:
L¹ is C₁₋₃ alkylene;
each L² is independently selected from C₁₋₃ alkylene, O, and N(R^{N});
n is 0, 1, or 2; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are each independently selected from H, halo, C₁₋₃ alkyl, C₁₋₃ alkoxy, C₁₋₃ haloalkyl, C₁₋₃ haloalkoxy, and C₃₋₁₀ cycloalkyl-C₁₋₃ alkyl.

18. The compound of claim 16, wherein the compound of Formula (II) has formula: 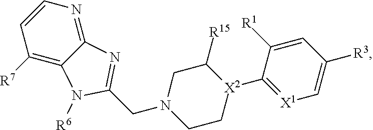
or a pharmaceutically acceptable salt thereof, wherein:
R¹⁵ is selected from H and C₁₋₃ alkyl;
X¹ is selected from N and CH;
X² is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl, C₁₋₃ haloalkyl, or HO—C₁₋₃ alkyl; and
R⁷ is H, or R⁷ and R⁶ together with the atoms to which they are attached form a 6-membered heterocycloalkyl ring.

19. The compound of claim 18, wherein the compound is selected from: 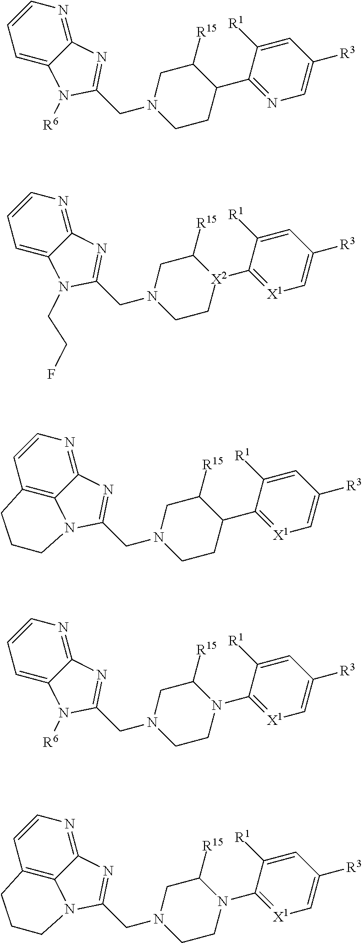
or a pharmaceutically acceptable salt thereof.

20. The compound of claim 16, wherein the compound of Formula (II) has formula: 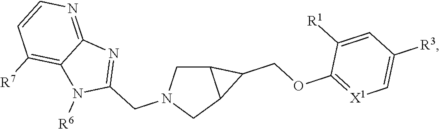
or a pharmaceutically acceptable salt thereof, wherein:
X¹ is selected from N and CH;
R¹ and R³ are each independently selected from halo, C₁₋₃ alkoxy, C₁₋₃ haloalkyl, and C₁₋₃ haloalkoxy;
R⁶ is C₁₋₃ alkyl; and
R⁷ is H, or R⁷ and R⁶ together with the atoms to which they are attached form a 6-membered heterocycloalkyl ring.

21. The compound of claim 20, wherein the compound is selected from: 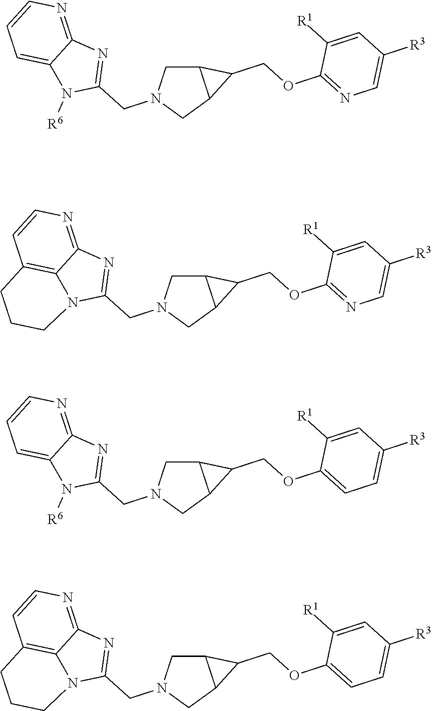
or a pharmaceutically acceptable salt thereof.

22. The compound of claim 16, wherein the compound of Formula (II) has formula: 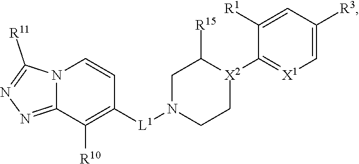
or a pharmaceutically acceptable salt thereof, wherein:
L¹ is selected from CH₂ and C(═O);
R¹¹ is C₃₋₁₀ cycloalkyl-C₁₋₃ alkyl;
R¹⁰ is selected from halo and C₁₋₃ haloalkyl;
R¹⁵ is selected from H and C₁₋₃ alkyl;
X² is selected from N and CH; and
R¹ and R³ are each independently selected from halo, C₁₋₃ alkoxy, C₁₋₃ haloalkyl, and C₁₋₃ haloalkoxy.

23. The compound of claim 22, wherein the compound is selected from: 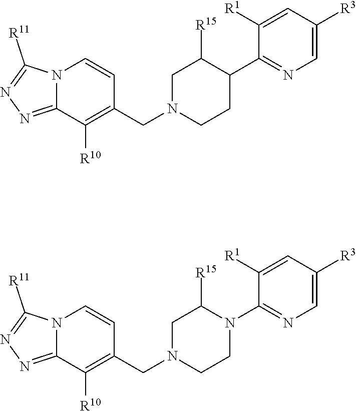
or a pharmaceutically acceptable salt thereof.

24. The compound of claim 16, wherein the compound of Formula (II) has formula: 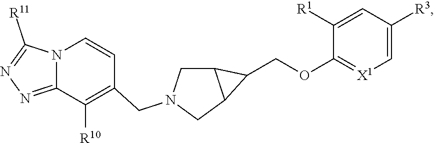
or a pharmaceutically acceptable salt thereof, wherein:
R¹¹ is C₃₋₁₀ cycloalkyl-C₁₋₃ alkyl;
R¹⁰ is selected from halo and C₁₋₃ haloalkyl;
X¹ is selected from N and CH; and
R¹ and R³ are each independently selected from halo, C₁₋₃ alkoxy, C₁₋₃ haloalkyl, and C₁₋₃ haloalkoxy.

25. The compound of claim 16, wherein the compound of Formula (II) is selected from: 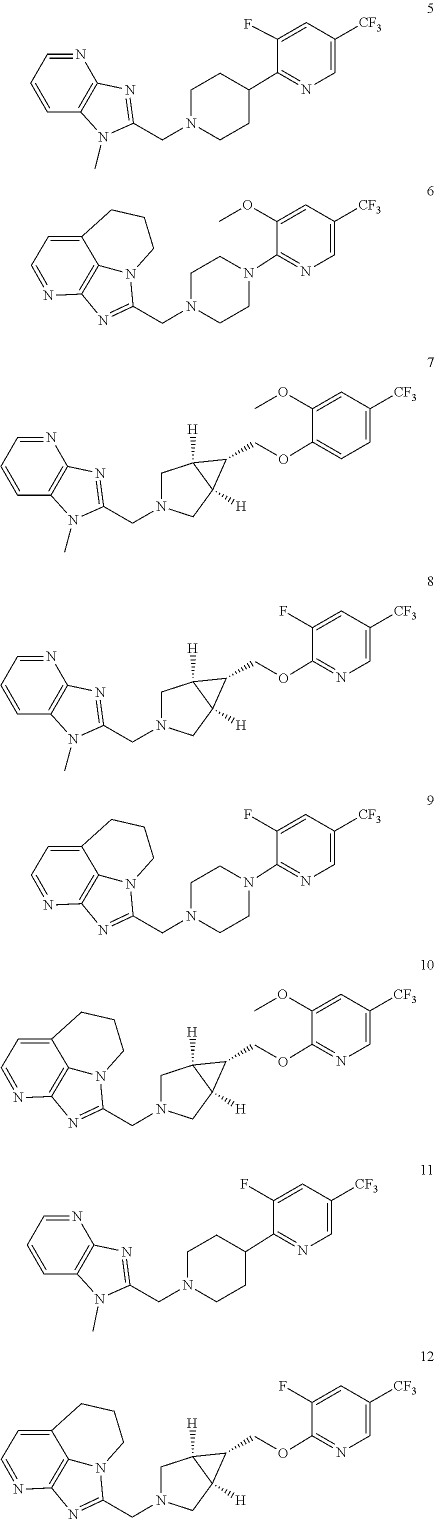 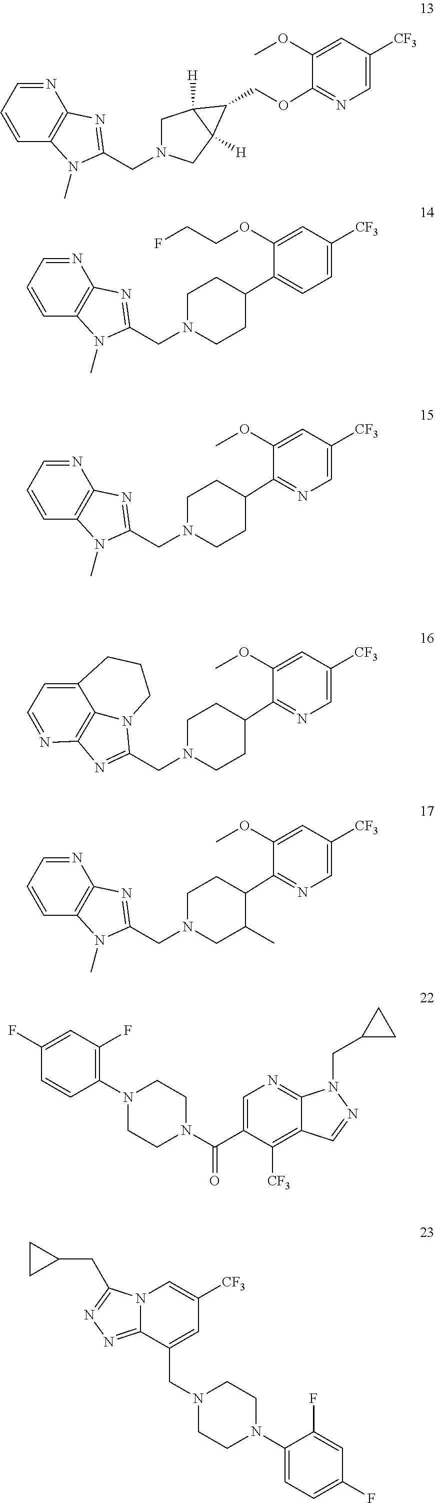
or a pharmaceutically acceptable salt thereof.

26. A pharmaceutical composition comprising a compound of claim 16, or a pharmaceutically acceptable salt thereof, and a pharmaceutically acceptable carrier.

27. A method of treating a psychiatric or a neurological disorder associated with mGluR2 in a subject, the method comprising administering to the subject in need thereof a therapeutically effective amount of a compound of claim 16, or a pharmaceutically acceptable salt thereof.

28. The method of claim 27, wherein the neurological disorder associated with mGluR2 is selected from Alzheimer's disease, Parkinson's disease, dyskinesia, Lewy body disease, Prion disease, motor neuron disease (MND), and Huntington's disease.

29. The method of claim 27, wherein the psychiatric disorder is selected from schizophrenia, psychosis, anxiety, depression, drug abuse, pain, smoking cessation, and epilepsy.


SEQUENCE LISTING (1 sequence)

SEQ ID NO: 1 - protein, 872 aa, Unknown [sequence omitted]